(19) United States
(12) Patent Application Publication
(10) Pub. No.: US 2024/0239794 A1
(43) Pub. Date: Jul. 18, 2024

(54) PKC-Theta Modulators

(71) Applicant: Exscientia AI Limited, Oxford (GB)

(72) Inventors: Peter Ray, Oxford (GB); Anthony Bradley, Oxford (GB); Simon Richards, Oxford (GB); Catarina Santos, Oxford (GB); Jeremy Besnard, Oxford (GB); Jérôme Meneyrol, Toulouse (FR); Virginie Suchaud, Toulouse (FR)

(73) Assignee: Exscientia AI Limited, Oxford (GB)

(21) Appl. No.: 18/557,865

(22) PCT Filed: May 6, 2022
(86) PCT No.: PCT/GB2022/051167

§ 371 Date: Oct. 27, 2023

(30) Foreign Application Priority Data
May 6, 2021 (GB) 2106486.0

(51) Int. Cl. C07D 471/04; A61K 31/52

(52) U.S. Cl. CPC C07D 471/04; A61K 31/52

(57) ABSTRACT

Disclosed are compounds, compositions and methods for treating disease, syndromes, conditions and disorders that are affected by the modulation of PKC-theta. Such compounds are represented by Formula I, wherein the variables are defined herein. I

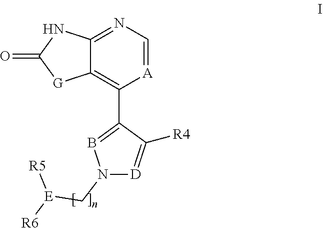

### TECHNICAL FIELD

The present disclosure relates to novel compounds capable of modulating PKC-theta phosphorylation activity. Such phosphorylation activity may be inhibited by the compounds described herein. The present invention further describes the synthesis of the compounds and their uses as medicaments in diseases or disorders where PKC-theta modulation may be beneficial.

### BACKGROUND

Protein kinases constitute a large family of structurally related enzymes that are responsible for the control of a variety of signal transduction processes within the cell (see Hardie, G and Hanks, S. The Protein Kinase Facts Book, I and II, Academic Press, San Diego, CA: 1995).

The connection between abnormal protein phosphorylation and disease is well known. Accordingly, protein kinases are an important group of drug targets (see, for example, Cohen, Nature, vol. 1 (2002), pp 309-315, Gaestel et al. Curr. Med. Chem, 2007, pp 2214-223; Grimminger et al. Nat. Rev. Drug Disc. vol. 9(12), 2010, pp 956-970).

Protein kinase C (hereafter PKC) is a family of serine- and threonine-specific protein kinases. PKC family members phosphorylate a wide variety of protein targets and are known to be involved in diverse cellular signalling pathways. Each member of the PKC family has a specific expression profile and is believed to have a distinct role.

The PKC members can be classified into three groups. Group I (Ca2+ and DAG (diacylglycerol) dependent): PKC-alpha, PKC-βI, PKC-βII and PKC-γ, Group II (Ca2+ independent): PKC-δ (hereafter PKC-delta), PKC-e, PKC-η (or PKC-eta) and PKC-θ (hereafter PKC-theta). Group III (Ca2+ and DAG independent): PKC-i, PKC-ζ and PKC-μ (Brezar et al 2015 Frontiers Immunol).

The expression of the PKC-theta isoform of PKC is enriched in T lymphocytes and plays an important role in the T-cell receptor (TCR)-triggered activation of T-cells. PKC-theta signals through transcription factors, including NF-κB, NFAT and AP-1, leading to the release of cytokines such as IL-2 and IFN-gamma, and subsequently T-cell proliferation, differentiation and survival (Brezar et al 2015 Front Immunol). Unlike broader biommunosuppressive mechanisms, including those displayed by the calcineurin inhibitors, PKC-theta inhibition has demonstrated a selective effect on the immune system (Brezar et al 2015 Front Immunol 6:530). Antiviral responses remain intact in mice lacking PKC-theta activity (Zhang et al Adv Pharm. 2013; 66: 267-31). In regulatory T-cells (Tregs), PKC-theta signalling is not absolutely required for activation and function (Zhang et al. Adv Pharmacol. 2013; 66: 267-31). Prkcq^{-/-} mice have a reduced but significant proportion of circulating Tregs and Tregs isolated from Prkcq^{-/-} mice retain suppressive activity (Gupta, et al., 2008). Pharmacological inhibition of PKC-theta protected Tregs from inactivation by TNFα and enhanced protection of mice from inflammatory colitis (Zanin-Zhorov, et al., 2010). Indeed, evidence has emerged that PKC-theta is a negative regulator of Tregs function (Zhang et al Adv Pharm. 2013; 66: 267-31).

In human disease, associations of the Prkcq locus specific single nucleotide polymorphisms (SNP) have been identified with type 1 diabetes (T1D), rheumatoid arthritis (RA), and celiac disease by genome-wide association studies (GWAS; Brezar et al 2015 Front Immunol 6:530). Further, pharmacological inhibition of PKCθ rescued the defective activity of Tregs from rheumatoid arthritis patients (Zanin-Zhorov, et al., 2010).

PKC-theta activity is critically important in Th2 (allergic disease) and Th17 (autoimmune disease) responses and differentiation (Zhang et al Adv Pharm. 2013; 66: 267-31).). The Prkcq^{-/-} mouse is protected in Th2 models of allergic lung inflammation and parasite infection. Likewise, lack of PKC-theta activity is protective in Th17-driven mouse models such as experimental autoimmune encephalomyelitis (EAE), adjuvant-induced arthritis, and colitis.

PKC-theta is also implicated in various types of cancers and the PKC-theta-mediated signalling events controlling cancer initiation and progression. In these types of cancers, the high PKC-theta expression leads to aberrant cell proliferation, migration and invasion resulting in malignant phenotype (Nicolle, A et al., Biomolecules, 2021, 11, 221. Inhibition of PKC-theta may also benefit the treatment for cancers in which PKC-theta has been implicated.

Small molecule inhibitors of PKC-theta are known, for example inhibitors based on a pyrazolopyrimidine scaffold are described in WO 2011/139273, and WO 2015/095679 describes PKC-theta inhibitors based on a diaminopyrimidine core.

To date there is no effective and approved medical treatment available which is based on the inhibition of PKC-theta, largely due to the difficulties of securing potent inhibition alongside suitable selectivity for the PKC-theta isoform over other isoforms, particularly PKC-delta in the PKC family (Group 2), and other kinases.

The present invention has been devised with the above observations in mind.

### SUMMARY OF THE INVENTION

In one aspect of the invention there is provided a compound of Formula I:

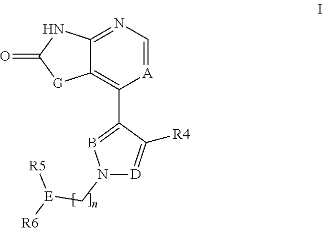

or a pharmaceutically acceptable salt, solvate, stereoisomer or mixture of stereoisomers, tautomer, or isotopic form, or pharmaceutically active metabolite thereof, or combinations thereof, wherein:
A is selected from the group consisting of: N, C—R^{a}, where R^{a} is selected from hydrogen, halogen, C1-3 alkyl and CN;
G is selected from the group consisting of: CR1R2; O and NR1;
R1 and R2 are independently selected from the group consisting of: hydrogen, halogen, C1-3 alkyl; C3-7 cycloalkyl (e.g. CH₂^{c}Pr); C1-3 alkoxyl (e.g. OMe); C2-6 cycloalkoxyl (e.g. O^{c}Pr); C2-6 alkyl alkoxy (e.g. CH₂OMe), hydroxyl, C1-3 alkyl hydroxyl (e.g. CH₂OH), amino, C1-3 alkyl amino (e.g. CH₂NH₂); C1-4 amino alkyl (e.g. NHMe or N(Me)₂), C2-7 alkyl amino alkyl (e.g. CH₂NHMe or CH₂N(Me)₂); and C1-3 haloalkyl; or
R1 and R2 together form a 3-5 membered optionally substituted spiro carbocyclic or heterocyclic ring; particularly a 4-5 membered optionally substituted carbocyclic or heterocyclic spiro ring; wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxy; and C1-2 alkoxy;
B is selected from the group consisting of: N; C—H and C-halogen (e.g. C—F, C—Cl, C—Br);
D is selected from the group consisting of: N and C—R3;
R3 is selected from the group consisting of: hydrogen; C1-3 alkyl (e.g. Me, Et); C1-3 halo alkyl (e.g. CF₂H, CF₃; CH₂CF₃); C1-3 alkoxyl (e.g. OMe); C2-5 alkyl alkoxyl (e.g. CH₂OMe); and halogen (e.g. F, Cl, Br); and
R4 is selected from the group consisting of: hydrogen; C1-3 alkyl; C1-3 halo alkyl (e.g. CF₂H, CF₃; CH₂CF₃); OMe and halogen; or
wherein when D is C—R3, R3 and R4 together are joined to form an optionally substituted aryl or heteroaryl ring having the structure selected from the group consisting of:

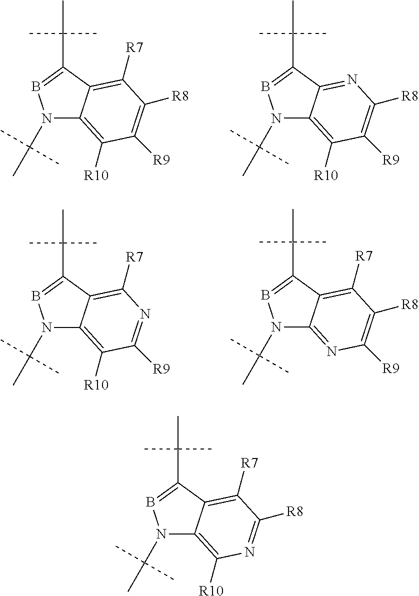

wherein;
R7 is selected from the group consisting of: hydrogen and halogen;
R8 is selected from the group consisting of: hydrogen and halogen;
R9 is selected from the group consisting of: hydrogen; C1-3 halo alkyl (e.g. CF₂H, CF₃; CH₂CF₃) and halogen;
R10 is selected from the group consisting of: hydrogen; halogen; C1-3 halo alkyl (e.g. CF₂H, CF₃; CH₂CF₃); and C1-3 haloalkoxy (e.g. OCFH₂, OCF₂H, OCF₃); and wherein:
n is selected from the group consisting of: 0 and 1;
E is selected from the group consisting of: C—H; and C—R^{a}, where R^{a} is selected from halogen; C1-3 alkyl; C1-3 alkyl hydroxyl (e.g. CH₂OH); C1-3 haloalkyl (e.g. CH₂F); C2-6 alkyl alkoxyl (e.g. CH₂OMe) and C2-4 alkyl nitrile (e.g. CH₂CN);
R5 and R6 are joined together to form an optionally substituted, optionally bridged, 4-8-membered, suitably 5-7-membered, saturated carbocyclic or heterocyclic ring.

In embodiments, the compound according to the disclosure has the structural Formula II:

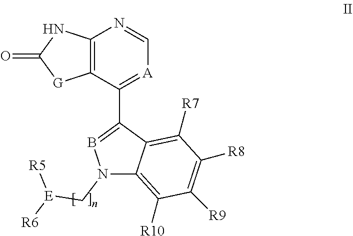

In embodiments, the compound according to the disclosure has the structural Formula IIa:

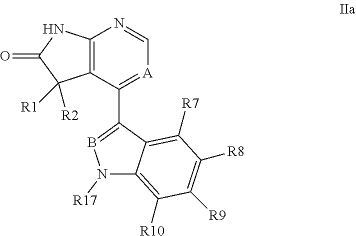

wherein, R17 is selected from the group consisting of:

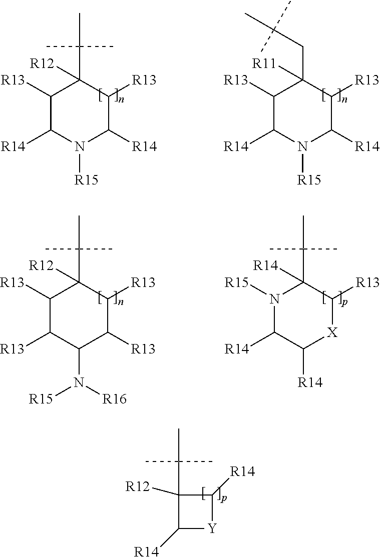

wherein
R11 is selected from the group consisting of: hydrogen; halogen and C1-2 alkyl;
R12 is selected from the group consisting of: hydrogen; C1-3 alkyl; C1-3 haloalkyl; C1-3 alkyl hydroxyl; and C1-2 alkyl nitrile;
R13 is selected from the group consisting of: hydrogen; halogen and C1-2 alkyl;
R14 is selected from the group consisting of: hydrogen and C1-2 alkyl;
R15 is selected from the group consisting of: hydrogen and C1-2 alkyl;
R16 is selected from the group consisting of: hydrogen; C1-3 alkyl; C1-3 haloalkyl; C1-3 alkyl hydroxy; and C1-3 alkyl alkoxy;
n is selected from the group consisting of: 0 and 1;
p is selected from the group consisting of: 1 and 2;
X is selected from the group consisting of: CH₂ and O;
Y is selected from the group consisting of: CH₂; O; NH and NMe.

In embodiments:
R1 is selected from the group consisting of: hydrogen, Me, Et, OMe, OEt, OH, NH₂, NHMe and NHEt;
R2 is selected from the group consisting of: hydrogen, Me and Et; or
R1 and R2 together form a 3-5 membered optionally substituted spiro carbocyclic or heterocyclic ring; particularly a 4-5 membered optionally substituted carbocyclic or heterocyclic spiro ring; wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxyl; and C1-2 alkoxyl;
A is selected from the group consisting of: C—H; C—F; C—Cl and C—Br;
B is selected from the group consisting of: N; C—H, C—F; C—Cl and C—Br;
R17 is selected from the group consisting of:

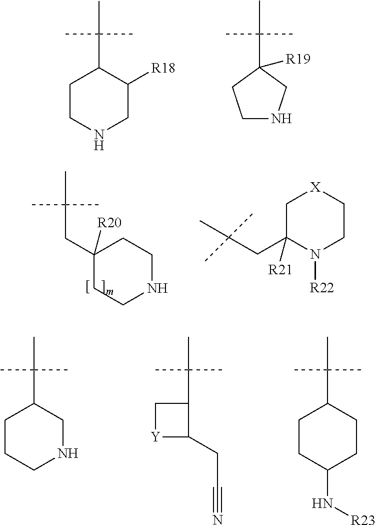

wherein:
R18 is selected from the group consisting of: hydrogen; and halogen;
R19 is selected from the group consisting of: hydrogen; C1-3 alkyl; C1-3 haloalkyl; C1-3 alkyl hydroxy;
m is selected from the group consisting of: 0 and 1;
R20 is selected from the group consisting of: hydrogen; halogen;
X is selected from the group consisting of: CH₂ and O;
R21 and R22 are each independently selected from the group consisting of: hydrogen and C1-3 alkyl;
Y is selected from the group consisting of: CH₂; O and NH;
R23 is selected from the group consisting of: hydrogen; C1-3 alkyl; C1-3 haloalkyl.

In embodiments, the compound according to the disclosure has the structural Formula III:

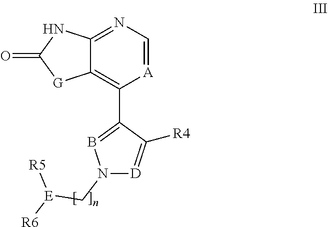

wherein;
D is selected from the group consisting of: N; C—H and C—R3;
R3 is selected from the group consisting of: C1-3 alkyl; C2-5 alkyl alkoxy; C1-3 haloalkyl and halogen;
R4 is selected from the group consisting of: hydrogen; C1-3 alkyl; C2-5 alkyl alkoxyl; C1-3 haloalkyl and halogen.

In embodiments, the compound according to the disclosure has the structural Formula IIIa, IIIb or IIIc:

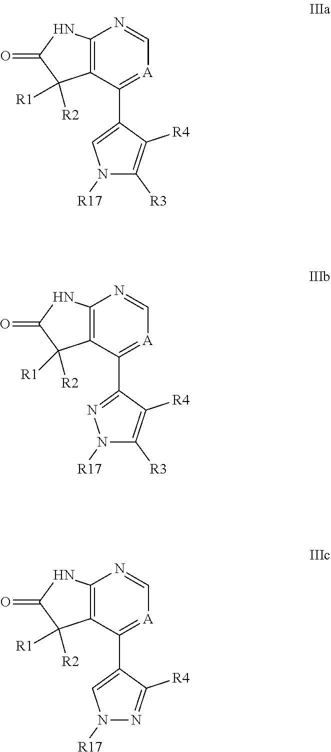

wherein,
R17 is selected from the group consisting of:

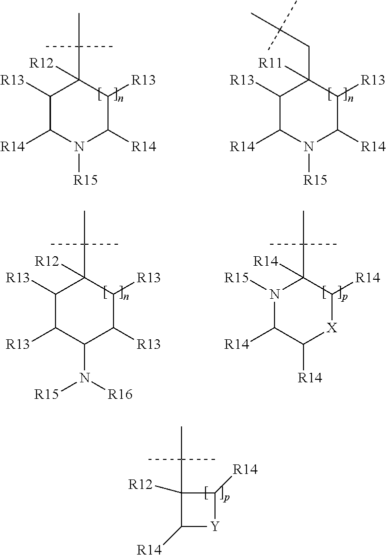

wherein
R11 is selected from the group consisting of: hydrogen; halogen and C1-2 alkyl;
R12 is selected from the group consisting of: hydrogen; C1-3 alkyl; C1-3 haloalkyl; C1-3 alkyl hydroxyl; and C1-2 alkyl nitrile;
R13 is selected from the group consisting of: hydrogen; halogen and C1-2 alkyl;
R14 is selected from the group consisting of: hydrogen and C1-2 alkyl;
R15 is selected from the group consisting of: hydrogen and C1-2 alkyl;
R16 is selected from the group consisting of: hydrogen; C1-3 alkyl; C1-3 haloalkyl; C1-3 alkyl hydroxyl; and C1-3 alkyl alkoxyl;
n is selected from the group consisting of: 0 and 1;
p is selected from the group consisting of: 1 and 2;
X is selected from the group consisting of: CH₂ and O;
Y is selected from the group consisting of: CH₂, O, NH and NMe.

In embodiments,
R1 is selected from the group consisting of: hydrogen, Me, Et, OMe, OH, NH₂, and NHMe;
R2 is selected from the group consisting of: hydrogen; Me; and Et; or
R1 and R2 together form a 3-5 membered optionally substituted spiro carbocyclic or heterocyclic ring;
A is selected from the group consisting of: C—H, C—F, C—Cl and C—Br;
R17 is selected from the group consisting of:

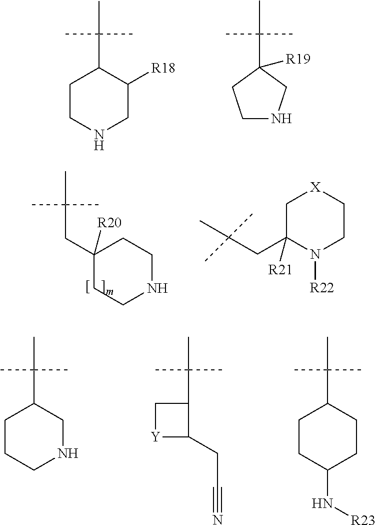

wherein:
R18 is selected from the group consisting of: hydrogen and halogen;
R19 is selected from the group consisting of: hydrogen; C1-3 alkyl; C1-3 haloalkyl; C1-3 alkyl and hydroxyl;
m is selected from the group consisting of: 0 and 1;
R20 is selected from the group consisting of: hydrogen and halogen;
X is selected from the group consisting of: CH₂; and O;
R21 and R22 are each independently selected from the group consisting of: hydrogen and C1-3 alkyl;
Y is selected from the group consisting of: CH₂; O and NH;
R23 is selected from the group consisting of: hydrogen; C1-3 alkyl; and C1-3 haloalkyl.

In another aspect the invention provides a pharmaceutical composition comprising a compound according to this disclosure or a pharmaceutically acceptable salt, solvate, stereoisomer or mixture of stereoisomers, tautomer, or isotopic form, or pharmaceutically active metabolite thereof, or combinations thereof, and one or more pharmaceutically acceptable carrier.

In another aspect the invention provides the compound according to this disclosure or the pharmaceutical composition according to this disclosure for use in the treatment of a disorder or disease selected from autoimmune disorders and/or inflammatory diseases and/or oncologic disease and/or cancers and/or HIV infection and replication. Suitably, the disorder or disease is selected from the group consisting of: rheumatoid arthritis, multiple sclerosis, psoriasis, atopic dermatitis.

In embodiments, the compound or pharmaceutical composition for use according to this disclosure is an inhibitor of PKC-theta.

In embodiments, the use is in a method comprising administering the compound orally, topically, by inhalation, by intranasal administration, or systemically by intravenous, intraperitoneal, subcutaneous, or intramuscular injection. In embodiments, the use is in a method comprising administering the compound according to this disclosure in combination with one or more additional therapeutic agents. In embodiments, the administering comprises administering the compound according to this disclosure simultaneously, sequentially or separately from the one or more additional therapeutic agent.

In embodiments, the use comprises administering to a subject an effective amount of the compound according to this disclosure, wherein the effective amount is between about 5 nM and about 10 μM in the blood of the subject.

In another aspect of the invention there is provided a method of treating or preventing PKC-theta mediated disorders, or a condition treatable or preventable by inhibition of a kinase, for example, PKC-theta. In embodiments, the disease may be a disease associated with autoimmunity, inflammatory disease, cancer and/or oncologic disease and/or oncologic disease and/or cancers and/or HIV infection and replication (particularly autoimmune disorders and inflammatory diseases) in a subject in need thereof. Suitably, the disorder or disease is selected from the group consisting of: rheumatoid arthritis, multiple sclerosis, psoriasis, atopic dermatitis.

In embodiments, the method comprises administering a compound according to this disclosure or a pharmaceutical composition according to this disclosure. Suitably the compound is, or the pharmaceutical composition comprises, an inhibitor of PKC-theta.

In embodiments, the method comprises administering the compound or pharmaceutical composition orally, topically, by inhalation, by intranasal administration, or systemically by intravenous, intraperitoneal, subcutaneous, or intramuscular injection. In embodiments, method comprises administering the compound according to this disclosure or pharmaceutical composition according to this disclosure in combination with one or more additional therapeutic agents. In embodiments, the administering comprises administering the compound according to this disclosure or pharmaceutical composition according to this disclosure simultaneously, sequentially or separately from the one or more additional therapeutic agent.

In embodiments, the method comprises administering to a subject an effective amount of the compound according to this disclosure, wherein the effective amount is between about 5 nM and about 10 μM in the blood of the subject.

Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. More particularly, it is specifically intended that any embodiment of any aspect may form an embodiment of any other aspect, and all such combinations are encompassed within the scope of the invention. The applicant reserves the right to change any originally filed claim or file any new claim, accordingly, including the right to amend any originally filed claim to depend on and/or incorporate any feature of any other claim although not originally claimed in that manner.

### DETAILED DESCRIPTION

Described herein are compounds and compositions (e.g., organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from PKC-theta modulation include, for example, autoimmune disorder, inflammatory disease, cancer and/or oncologic disease and/or HIV infection and replication, such as rheumatoid arthritis, multiple sclerosis, psoriasis, asthma, atopic dermatitis and Crohn's disease.

The compounds may also or alternatively be useful as lead molecules for the selection, screening and development of further derivatives that may have one or more improved beneficial drug property, as desired. Such further selection and screening may be carried out using the proprietary computational evolutionary algorithm described e.g. in the Applicant's earlier published patent application WO 2011/061548, which is hereby incorporated by reference in its entirety.

The disclosure also encompasses salts, solvates and functional derivatives of the compounds described herein. These compounds may be useful in the treatment of diseases or disorders which may benefit from PKC-theta modulation, such as the autoimmune disorders, inflammatory diseases, cancers and/or oncologic diseases and/or HIV infection and replication identified herein.

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present invention employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety.

Prior to setting forth the detailed description of the invention, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising the compounds of the disclosure.

Prodrugs and solvates of the compounds of the disclosure are also encompassed within the scope of the disclosure. The term ‘prodrug’ means a compound (e.g. a drug precursor) that is transformed in vivo to yield a compound of the disclosure or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A.C.S. Symposium Series; (1987), “Bioreversible Carriers in Drug Design”, Roche, ed., American Pharmaceutical Association and Pergamon Press). The compositions and medicaments of the disclosure therefore may comprise prodrugs of the compounds of the disclosure. In some aspects and embodiments the compounds of the disclosure are themselves prodrugs which may be metabolised in vivo to give the therapeutically effective compound.

The invention also includes various deuterated forms of the compounds of any of the Formulas disclosed herein, including Formulas (I), (II), or (III) (inc. corresponding subgeneric formulas defined herein), respectively, or a pharmaceutically acceptable salt and/or a corresponding tautomer form thereof (including subgeneric formulas, as defined above) of the present invention. Each available hydrogen atom attached to a carbon atom may be independently replaced with a deuterium atom. A person of ordinary skill in the art will know how to synthesize deuterated forms of the compounds of any of the Formulas disclosed herein, including Formulas (I), (II), or (III) (inc. corresponding subgeneric formulas defined herein), respectively, or a pharmaceutically acceptable salt and/or a corresponding tautomer form thereof (including subgeneric formulas, as defined above) of the present invention. For example, deuterated materials, such as alkyl groups may be prepared by conventional techniques (see for example: methyl-d3-amine available from Aldrich Chemical Co., Milwaukee, WI, Cat. No. 489,689-2).

The subject invention also includes isotopically-labelled compounds which are identical to those recited in any of the Formulas disclosed herein, including Formulas (I), (II), or (III) (inc. corresponding subgeneric formulas defined herein), respectively, or a pharmaceutically acceptable salt and/or a corresponding tautomer form thereof (including subgeneric formulas, as defined above) of the present invention but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as 3H, 11C, 14C, 18F, 123I or 125I. Compounds of the present invention and pharmaceutically acceptable salts of said compounds that contain the aforementioned isotopes and/or other isotopes of other atoms are within the scope of the present invention. Isotopically labelled compounds of the present invention, for example those into which radioactive isotopes such as 3H or 14C have been incorporated, are useful in drug and/or substrate tissue distribution assays. Tritiated, i.e. 3H, and carbon-14, i.e. 14C, isotopes are particularly preferred for their ease of preparation and detectability. 11C and 18F isotopes are particularly useful in PET (positron emission tomography).

In the context of the present disclosure, the terms ‘individual’, ‘subject’, or ‘patient’ are used interchangeably to indicate an animal that may be suffering from a medical (pathological) condition and may be responsive to a molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-12 alkyl (or C1-12 alkyl) refers to any alkyl group containing 1 to 12 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described below.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this invention and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. By ‘optionally substituted’ it is meant that the group concerned is either unsubstituted, or at least one hydrogen atom is replaced with one of the specified substituent groups, radicals or moieties.

Any radical/group/moiety described herein that may be substituted (or optionally substituted) may be substituted with one or more (e.g. one, two, three, four or five) substituents, which are independently selected from the designated group of substituents. Thus, substituents may be selected from the group: halogen (or ‘halo’, e.g. F, Cl and Br), hydroxyl (—OH), amino or aminyl (—NH₂), thiol (—SH), cyano (—CN), (lower) alkyl, (lower) alkoxy, (lower) alkenyl, (lower) alkynyl, aryl, heteroaryl, (lower) alkylthio, oxo, haloalkyl, hydroxyalkyl, nitro (—NO₂), phosphate, azido (—N₃), alkoxycarbonyl, carboxy, alkylcarboxy, alkylamino, dialkylamino, aminoalkyl, alkylaminoalkyl, dialkylaminoalkyl, thioalkyl, alkylsulfonyl, arylsulfinyl, alkylaminosulfonyl, arylaminosulfonyl, alkylsulfonylamino, arylsulfonylamino, carbamoyl, alkylcarbamoyl, dialkylcarbamoyl, arylcarbamoyl, alkylcarbonylamino, arylcarbonylamino, cycloalkyl, heterocycloalkyl, unless otherwise indicated. Alternatively, where the substituents are on an aryl or other cyclic ring system, two adjacent atoms may be substituted with a methylenedioxy or ethylenedioxy group. More suitably, the substituents are selected from: halogen, hydroxy, amino, thiol, cyano, (C₁-C₆)alkyl, (C₁-C₆)alkoxy, (C₁-C₆)alkenyl, (C₁-C₆)alkynyl, aryl, aryl(C₁-C₆)alkyl, aryl(C₁-C₆)alkoxy, heteroaryl, (C₁-C₆)alkylthio, oxo, halo(C₁-C₆)alkyl, hydroxy(C₁-C₆)alkyl, nitro, phosphate, azido, (C₁-C₆)alkoxycarbonyl, carboxy, (C₁-C₆)alkylcarboxy, (C₁-C₆)alkylamino, di(C₁-C₆)alkylamino, amino(C₁-C₆)alkyl, (C₁-C₆)alkylamino(C₁-C₆)alkyl, di(C₁-C₆)alkylamino(C₁-C₆)alkyl, thio(C₁-C₆)alkyl, (C₁-C₆)alkylsulfonyl, arylsulfinyl, (C₁-C₆)alkylaminosulfonyl, arylaminosulfonyl, (C₁-C₆)alkylsulfonylamino, arylsulfonylamino, carbamoyl, (C₁-C₆)alkylcarbamoyl, di(C₁-C₆)alkylcarbamoyl, arylcarbamoyl, (C₁-C₆)alkylcarbonylamino, arylcarbonylamino, (C₁-C₆)cycloalkyl, and heterocycloalkyl. Still more suitably, the substituents are selected from one or more of: fluoro, chloro, bromo, hydroxy, (C₁-C₆)alkyl, (C₁-C₆)haloalkyl, (C₁-C₆)alkoxy, (C₅-C₆)aryl, a 5- or 6-membered heteroaryl, (C4-C6)cycloalkyl, a 4- to 6-membered heterocycloalkyl, cyano, (C₁-C₆)alkylthio, amino, —NH(alkyl), —NH((C₁-C₆)cycloalkyl), —N((C₁-C₆)alkyl)₂, —OC(O)—(C₁-C₆)alkyl, —OC(O)—(C₅-C₆)aryl, —OC(O)—(C₁-C₆)cycloalkyl, carboxy and —C(O)O—(C₁-C₆)alkyl. Most suitably, the substituents are selected from one or more of: fluoro, chloro, bromo, hydroxy, amino, (C₁-C₆)alkyl and (C₁-C₆)alkoxy, wherein alkyl and alkoxy are optionally substituted by one or more chloro. Particularly preferred substituents are: chloro, methyl, ethyl, methoxy and ethoxy.

The term ‘halo’ or ‘halogen’ refers to a monovalent halogen radical chosen from chloro, bromo, iodo, and fluoro. A ‘halogenated’ compound is one substituted with one or more halo substituent. Preferred halo groups are F, Cl and Br, and most preferred is F.

When used herein, the term ‘independently’, in reference to the substitution of a parent moiety with one or more substituents, means that the parent moiety may be substituted with any of the listed substituents, either individually or in combination, and any number of chemically possible substituents may be used. In any of the embodiments, where a group is substituted, it may contain up to 5, up to 4, up to 3, or 1 and 2 substituents. As a non-limiting example, useful substituents include: phenyl or pyridine, independently substituted with one or more lower alkyl, lower alkoxy or halo substituents, such as: chlorophenyl, dichlorophenyl, trichlorophenyl, tolyl, xylyl, 2-chloro-3-methylphenyl, 2,3-dichloro-4-methylphenyl, etc.

As used herein, the term ‘alkylene’ or ‘alkylenyl’ means a difunctional group obtained by removal of a hydrogen atom from an alkyl group as defined above. Non-limiting examples of alkylene include methylene, ethylene and propylene. ‘Lower alkylene’ means an alkylene having from 1 to 6 carbon atoms in the chain, and may be straight or branched. Alkylene groups are optionally substituted.

The term ‘alkenyl’ refers to a monovalent, optionally substituted, unsaturated aliphatic hydrocarbon radical. Therefore, an alkenyl has at least one carbon-carbon double bond (C═C). The number of carbon atoms in the alkenyl group may be indicated, such as from 2 to about 20. For example, a C2-12 alkenyl (or C₂₋₁₂ alkenyl) refers to an alkenyl group containing 2 to 12 carbon atoms in the structure. Alkenyl groups may be straight (i.e. linear), branched chain, or cyclic. ‘Lower alkenyl’ refers to an alkenyl of 1 to 6 carbon atoms, and may have from 1 to 4 carbon atoms, or 1 to 2 carbon atoms. Representative examples of lower alkenyl radicals include ethenyl, 1-propenyl, 1-butenyl, 1-pentenyl, 1-hexenyl, isopropenyl, isobutenyl, and the like. Higher alkenyl refers to alkenyls of seven carbons and above, such as 1-heptenyl. 1-octenyl, 1-nonenyl, 1-decenyl, 1-dodecenyl, 1-tetradecenyl, 1-hexadecenyl, 1-octadecenyl, 1-eicosenyl, and the like, along with branched variations thereof. Optional substituents include are described elsewhere.

‘Alkenylene’ means a difunctional group obtained by removal of a hydrogen from an alkenyl group that is defined above. Non-limiting examples of alkenylene include —CH═CH—, —C(CH₃)═CH—, and —CH═CHCH₂—

‘Alkynyl’ and ‘lower alkynyl’ is defined similarly to the term ‘alkenyl’, except that it includes at least one carbon-carbon triple bond.

The term ‘alkoxy’ refers to a monovalent radical of the formula RO—, where R is any alkyl, alkenyl or alkynyl as defined herein. Alkoxy groups may be optionally substituted by any of the optional substituents described herein. ‘Lower alkoxy’ has the formula RO—, where the R group is a lower alkyl, alkenyl or alkynyl. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Preferred alkoxy groups are methoxy and ethoxy.

The term ‘aryl’ as used herein refers to a substituted or unsubstituted aromatic carbocyclic radical containing from 5 to about 15 carbon atoms; and preferably 5 or 6 carbon atoms. An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxy’ group, therefore, is a radical of the formula RO—, wherein R is a phenyl radical. ‘Benzyl’ is a radical of the formula R—CH₂—, wherein R is phenyl, and ‘benzyloxy’ is a radical of the formula RO—, wherein R is benzyl. Non-limiting examples of aryl radicals include, phenyl, naphthyl, benzyl, biphenyl, furanyl, pyridinyl, indanyl, anthraquinonyl, tetrahydronaphthyl, a benzoic acid radical, a furan-2-carboxylic acid radical, and the like.

A ‘heteroaryl’ group is herein defined as a substituted or unsubstituted ‘aryl’ group in which one or more carbon atoms in the ring structure has been replaced with a heteroatom, such as nitrogen, oxygen or sulphur. Generally, the heteroaryl group contains one or two heteroatoms. A preferred heteroatom is N. Exemplary heteroaryl groups include: furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinazoline, pyridazine and cinnoline.

The terms ‘heterocycle’ or ‘heterocyclic’ group as used herein refer to a monovalent radical of from about 4- to about 15-ring atoms, and preferably 4-, 5- or 6,7-ring members. Generally the heterocyclic group contains one, two or three heteroatoms, selected independently from nitrogen, oxygen and sulphur. A preferred heteroatom is N. A heterocyclic group may have only one individual ring or may comprise one or more fused rings in which at least one ring contains a heteroatom. It may be fully saturated or partially saturated and may be substituted or unsubstituted as in the case or aryl and heteroaryl groups. Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolidinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocycles with only one heteroatom include 2-, 3-, or 4-pyridinyl, 2H-pyranyl, and 4H-pyranyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity or through a linker such as an alkylene such as methylene or ethylene.

Unless defined otherwise, ‘room temperature’ is intended to mean a temperature of from about 18 to 28° C., typically between about 18 and 25° C., and more typically between about 18 and 22° C.

As used herein, the phrase ‘room temperature’ may be shortened to ‘rt’ or ‘RT’.

### Molecules and Compounds

Disclosed herein is a compound having the structural Formula I:

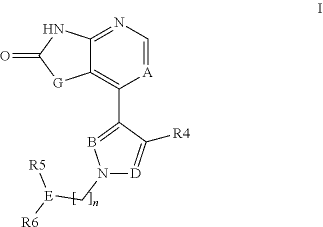

or a pharmaceutically acceptable salt, solvate, stereoisomer or mixture of stereoisomers, tautomer, or isotopic form, or pharmaceutically active metabolite thereof, or combinations thereof, wherein:
A is selected from the group consisting of: N, C—R^{a}, where R^{a} is selected from hydrogen, halogen, C1-3 alkyl and CN;
G is selected from the group consisting of: CR1R2; O and NR1;
R1 and R2 are independently selected from the group consisting of: hydrogen, halogen, C1-3 alkyl; C3-7 cycloalkyl (e.g. CH₂^{c}Pr); C1-3 alkoxyl (e.g. OMe); C2-6 cycloalkoxyl (e.g. O^{c}Pr); C2-6 alkyl alkoxy (e.g. CH₂OMe), hydroxyl, C1-3 alkyl hydroxyl (e.g. CH₂OH), amino, C1-3 alkyl amino (e.g. CH₂NH₂); C1-4 amino alkyl (e.g. NHMe or N(Me)₂), C2-7 alkyl amino alkyl (e.g. CH₂NHMe or CH₂N(Me)₂); C1-3 haloalkyl; or
R1 and R2 together form a 3-5 membered optionally substituted spiro carbocyclic or heterocyclic ring; particularly a 4-5 membered optionally substituted carbocyclic or heterocyclic spiro ring; wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxyl and C1-2 alkoxyl;
B is selected from the group consisting of: N; C—H and C-halogen (e.g. C—F, C—Cl, C—Br);
D is selected from the group consisting of: N and C—R3;
R3 is selected from the group consisting of: hydrogen; C1-3 alkyl (e.g. Me, Et); C1-3 halo alkyl (e.g. CF₂H, CF₃; CH₂CF₃); C1-3 alkoxyl (e.g. OMe); C2-5 alkyl alkoxyl (e.g. CH₂OMe); and halogen (e.g. F, Cl, Br); and
R4 is selected from the group consisting of: hydrogen; C1-3 alkyl; C1-3 halo alkyl (e.g. CF₂H, CF₃; CH₂CF₃); OMe and halogen; or
wherein when D is C—R3, R3 and R4 together are joined to form an optionally substituted aryl or heteroaryl ring having the structure selected from the group consisting of:

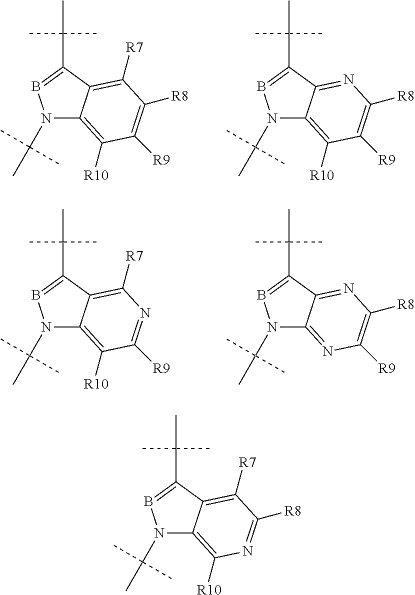

wherein;
R7 is selected from the group consisting of: hydrogen and halogen;
R8 is selected from the group consisting of: hydrogen and halogen;
R9 is selected from the group consisting of: hydrogen; C1-3 halo alkyl (e.g. CF₂H, CF₃; CH₂CF₃) and halogen;
R10 is selected from the group consisting of: hydrogen; halogen; C1-3 halo alkyl (e.g. CF₂H, CF₃; CH₂CF₃); C1-3 haloalkoxy (e.g. OCFH₂, OCF₂H, OCF₃); and
   wherein:
n is selected from the group consisting of: 0 and 1;
E is selected from the group consisting of: C—H; and C—R^{a}, where R^{a} is selected from halogen; C1-3 alkyl; C1-3 alkyl hydroxy (e.g. CH₂OH); C1-3 haloalkyl (e.g. CH₂F); C2-6 alkyl alkoxy (e.g. CH₂OMe) and C2-4 alkyl nitrile (e.g. CH₂CN);
R5 and R6 are joined together to form an optionally substituted, optionally bridged, 4-8-membered, suitably 5-7-membered, saturated carbocyclic or heterocyclic ring.

In specific embodiments of Formula I, D is C—R3, and R3 and R4 together are joined to form an optionally substituted aryl ring having the structure:

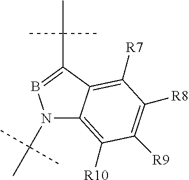

i.e. compounds of the general Formula II:

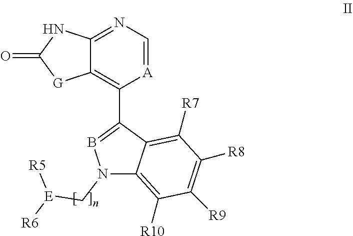

wherein, A, B, E, G, R1, R2, R5, R6, R7, R8, R9, R10 and n are defined as for Formula I.

In specific embodiments of Formula II, compounds have the structure of Formula IIa:

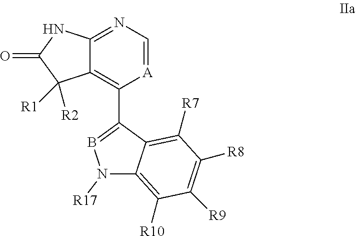

wherein, A, B, E, R1, R2, R5, R6, R7, R8, R9, R10 and n are defined as for Formula I;
R17 is selected from the group consisting of:

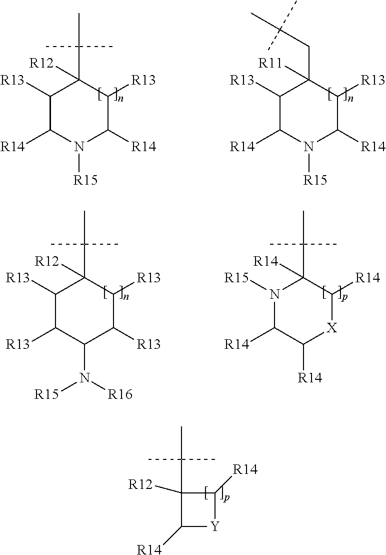

wherein
R11 is selected from the group consisting of: hydrogen; halogen (e.g. F) and C1-2 alkyl (e.g. Me);
R12 is selected from the group consisting of: hydrogen; C1-3 alkyl (e.g. Me); C1-3 haloalkyl (e.g. CH₂F); C1-3 alkyl hydroxy (e.g. CH₂OH); and C1-2 alkyl nitrile (e.g. CH₂CN);
R13 is selected from the group consisting of: hydrogen; halogen (e.g. F) and C1-2 alkyl (e.g. Me);
R14 is selected from the group consisting of: hydrogen and C1-2 alkyl (e.g. Me);
R15 is selected from the group consisting of: hydrogen and C1-2 alkyl (e.g. Me);
R16 is selected from the group consisting of: hydrogen; C1-3 alkyl (e.g. Me); C1-3 haloalkyl (e.g. —CH₂CH₂F, CH₂CHF₂, CH₂CF₃); C1-3 alkyl hydroxy (e.g. CH₂OH); and C1-3 alkyl alkoxyl (e.g. CH₂OMe);
n is selected from the group consisting of: 0 and 1;
p is selected from the group consisting of: 1 and 2;
X is selected from the group consisting of: CH₂ and O;
Y is selected from the group consisting of: CH₂, O, NH and NMe.

In specific embodiments of Formula IIa, E, R5, R6, R7, R8, R9 and R10 are as for Formula I;
R1 is selected from the group consisting of: hydrogen, Me, Et, OMe, OEt, OH, NH₂, NHMe and NHEt; and
R2 is selected from the group consisting of: hydrogen, Me and Et; or
R1 and R2 together form a 3-5 membered optionally substituted spiro carbocyclic or heterocyclic ring; particularly a 4-5 membered optionally substituted carbocyclic or heterocyclic spiro ring; wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxyl; and C1-2 alkoxyl;
A is selected from the group consisting of: C—H; C—F; C—Cl and C—Br;
B is selected from the group consisting of: N; C—H and C-halogen (e.g. C—F; C—Cl and C—Br);
R17 is selected from the group consisting of:

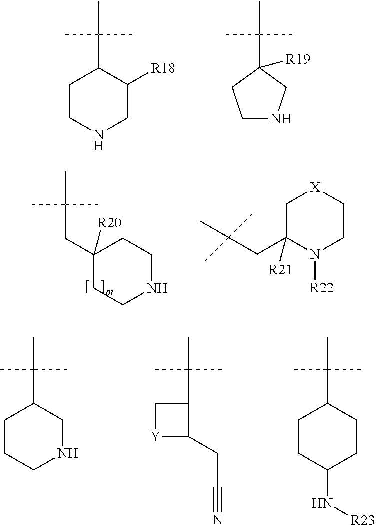

wherein:
R18 is selected from the group consisting of: hydrogen and halogen (e.g. F);
R19 is selected from the group consisting of: hydrogen; C1-3 alkyl (e.g. Me); C1-3 haloalkyl (e.g. CH₂F); C1-3 alkyl hydroxyl (e.g. CH₂OH);
m is selected from the group consisting of: 0 and 1;
R20 is selected from the group consisting of: hydrogen and halogen (e.g. F);
X is selected from the group consisting of: CH₂ and O;
R21 and R22 are each independently selected from the group consisting of: hydrogen; and C1-3 alkyl (e.g. Me);
Y is selected from the group consisting of: CH₂, O and NH;
R23 is selected from the group consisting of: hydrogen; C1-3 alkyl (e.g. Me) and C1-3 haloalkyl (e.g. —CH₂CH₂F, CH₂CHF₂, CH₂CF₃).

In alternative embodiments of Formula I, compounds have the general Formula III:

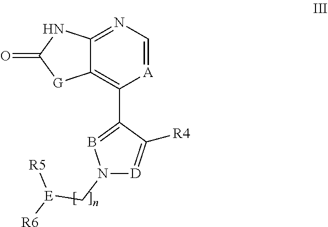

wherein, A, B, E, G, R1, R2, R5, R6 and n are defined as for Formula I, II, or IIa;
D is selected from the group consisting of: N; C—H and C—R3;
R3 is selected from the group consisting of: C1-3 alkyl; C2-5 alkyl alkoxy (e.g. OMe); C1-3 haloalkyl (e.g. CFs) and halogen;
R4 is selected from the group consisting of: hydrogen; C1-3 alkyl; C2-5 alkyl alkoxyl (e.g. OMe); C1-3 haloalkyl (e.g. CFs) and halogen.

In specific embodiments of Formula III, G is CR1R2 and one of B or D is N, the other being C—H; or B and D are C—H, i.e. compounds having the structure of Formula IIIa, IIIb or IIIc:

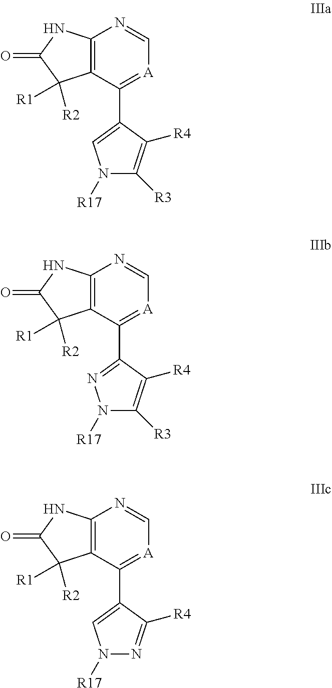

wherein, A, R1, R2, R3, R4, E, R5, R6 and n are defined as for Formula III;
R17 is selected from the group consisting of:

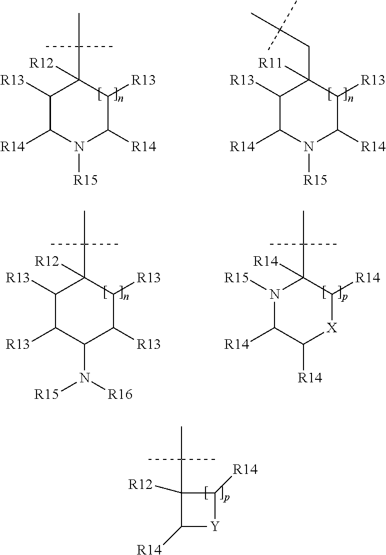

wherein
R11 is selected from the group consisting of: hydrogen; halogen (e.g. F) and C1-2 alkyl (e.g. Me);
R12 is selected from the group consisting of: hydrogen; C1-3 alkyl (e.g. Me); C1-3 haloalkyl (e.g. CH₂F); C1-3 alkyl hydroxy (e.g. CH₂OH); and C1-2 alkyl nitrile (e.g. CH₂CN)
R13 is selected from the group consisting of: hydrogen; halogen (e.g. F) and C1-2 alkyl (e.g. Me);
R14 is selected from the group consisting of: hydrogen and C1-2 alkyl (e.g. Me);
R15 is selected from the group consisting of: hydrogen and C1-2 alkyl (e.g. Me);
R16 is selected from the group consisting of: hydrogen; C1-3 alkyl (e.g. Me); C1-3 haloalkyl (e.g. —CH₂CH₂F, CH₂CHF₂, CH₂CF₃); C1-3 alkyl hydroxyl (e.g. CH₂OH); and C1-3 alkyl alkoxyl (e.g. CH₂OMe);
n is selected from the group consisting of: 0 and 1;
p is selected from the group consisting of: 1 and 2;
X is selected from the group consisting of: CH₂ and O;
Y is selected from the group consisting of: CH₂, O, NH and NMe.

In specific embodiments of Formula IIIa, IIIb and IIIc, A and n are as for Formula III, and
R1 is selected from the group consisting of: hydrogen, Me, Et, OMe, OH, NH₂, and NHMe; and
R2 is selected from the group consisting of: hydrogen; Me; and Et; or
R1 and R2 together form a 3-5 membered optionally substituted spiro carbocyclic or heterocyclic ring; particularly a 4-5 membered optionally substituted carbocyclic or heterocyclic spiro ring (e.g. cyclobutene, cyclopentane, tetrahydrofuran); wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxy; and C1-2 alkoxy;
A is selected from the group consisting of: C—H, C—F, C—Cl and C—Br;
R17 is selected from the group consisting of:

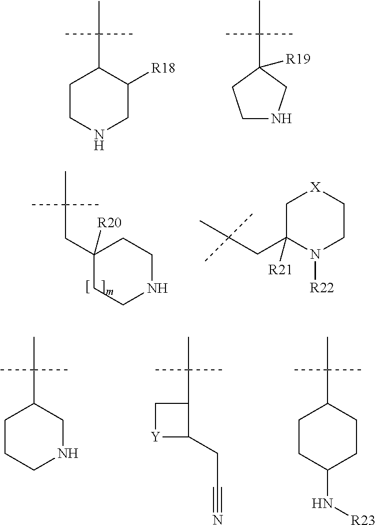

wherein:
R18 is selected from the group consisting of: hydrogen and halogen (e.g. F);
R19 is selected from the group consisting of: hydrogen; C1-3 alkyl (e.g. Me); C1-3 haloalkyl (e.g. CH₂F); and C1-3 alkyl hydroxy (e.g. CH₂OH);
m is selected from the group consisting of: 0 and 1;
R20 is selected from the group consisting of: hydrogen and halogen (e.g. F);
X is selected from the group consisting of: CH₂ and O;
R21 and R22 are each independently selected from the group consisting of: hydrogen and C1-3 alkyl (e.g. Me);
Y is selected from the group consisting of: CH₂; O and NH;
R23 is selected from the group consisting of: hydrogen; C1-3 alkyl (e.g. Me) and C1-3 haloalkyl (e.g. —CH₂CH₂F, CH₂CHF₂, CH₂CF₃).

In another aspect the invention provides a pharmaceutical composition comprising a compound according to this disclosure.

The compounds of the invention may have the structure as described below:

TABLE 1
Specific PKC-theta inhibitor compounds of the disclosure.

| Example | | Synthesis | | |
|---|---|---|---|---|
| no. | Structure | route | M + H | NMR |
| 1 | 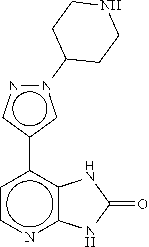 | pyrazole | 285.2 | 1H NMR (DMSO-d6, 500 MHz): δ (ppm) 11.52 (br d, J = 1.7 Hz, 1H), 10.95 (s, 1H), 8.78-9.19 (m, 2H), 8.51 (s, 1H), 8.18 (s, 1H), 7.86 (d, J = 5.6 Hz, 1H), 7.25 (d, J = 5.6 Hz, 1H), 4.53 (tt, J = 10.8, 4.1 Hz, 1H), 3.96 (br s, 1H), 3.40 (br d, J = 13.0 Hz, 2H), 3.07-3.16 (m, 2H), 2.25-2.33 (m, 2H), 2.13-2.24 (m, 2H) |
| 2 | 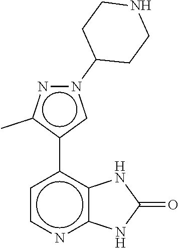 | pyrazole | 299.3 | 1H NMR (DMSO-d6, 500 MHz): δ (ppm) 11.45 (br s, 1H), 10.85 (br s, 1H), 9.07 (br s, 1H), 8.77 (br s, 1H), 8.05 (s, 1H), 7.88 (d, J = 5.6 Hz, 1H), 6.99 (d, J = 5.6 Hz, 1H), 4.39-4.47 (m, 1H), 4.22 (br s, 1H), 3,40 (br d, J = 13.0 Hz, 2H), 3.03-3.15 (m, 2H), 2.29 (s, 3H), 2.12-2.27 (m, 4H) |
| 3 | 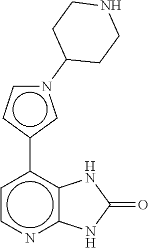 | pyrrole | 284.2 | 1H NMR (DMSO-d6, 500 MHz): δ (ppm) 11.77 (br s, 1H), 10.97 (br s, 1H), 9.08 (br s, 1H), 8.89 (br s, 1H), 7.82 (d, J = 5.9 Hz, 1H), 7.64 (s, 1H), 7.22 (d, J = 5.9 Hz, 1H), 7.00 (t, J = 2.2 Hz, 1H), 6.70 (br s, 1H), 4.28 (tt, J = 11.4, 3.6 Hz, 1H), 3.53 (br s, 1H), 3.36-3.46 (m, 2H), 3.05 (q, J = 12.1 Hz, 2H), 2.23 (br d, J = 12.5 Hz, 2H), 2.06-2.18 (m, 2H) |
| 4 | 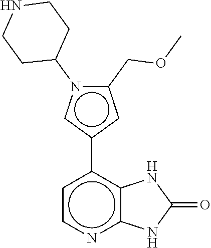 | pyrrole | 328.1 | 1H NMR (DMSO-d6, 600 MHz): δ (ppm) 11.24 (s, 1H), 10.76 (br s, 1H), 7.78 (d, J = 5.6 Hz, 1H), 7.69 (d, J = 1.9 Hz, 1H), 7.10 (d, J = 5.6 Hz, 1H), 6.62 (d, J = 1.9 Hz, 1H), 4.40 (s, 2H), 4.06 (tt, J = 11.7, 4.0 Hz, 1H), 3.22 (s, 3H), 3.06-3.16 (m, 2H), 2.65 (br t, J = 11.7 Hz, 2H), 2.68 (br s, 1H), 1.89-1.99 (m, 2H), 1.84 (br d, J = 10.0 Hz, 2H) |
| 5 | 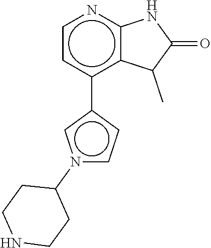 | pyrrole | 297.1 | 1H NMR (DMSO-d6, 500 MHz): δ (ppm) 11.18 (br s, 1H), 8.64-9.16 (m, 2H), 7.96 (d, J = 5.9 Hz, 1H), 7.50 (s, 1H), 7.15 (br d, J = 5.6 Hz, 1H), 6.98 (t, J = 2.4 Hz, 1H), 6.59 (br s, 1H), 4.31 (tt, J = 11.6, 4.0 Hz, 1H), 3.97 (q, J = 8.1 Hz, 1H), 3.83 (br s, 1H), 3.40 (br d, J = 12.7 Hz, 2H), 2.97-3.10 (m, 2H), 2.18-2.24 (m, 2H), 2.11 (br s, 2H), 1.27 (d, J = 7.3 Hz, 3H) |
| 6 | 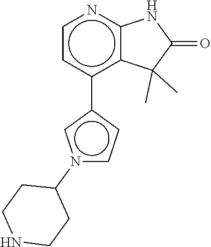 | pyrrole | 311.1 | 1H NMR (DMSO-d6, 500 MHz): δ (ppm) 10.95 (s, 1H), 8.45-8.94 (m, 2H), 7.97 (d, J = 5.6 Hz, 1H), 7.15 (t, J = 2.0 Hz, 1H), 6.96 (t, J = 2.4 Hz, 1H), 6.91 (d, J = 5.6 Hz, 1H), 6.34- 6.36 (m, 1H), 4.33 (tt, J = 11.6, 3.8 Hz, 1H), 3.41 (br d, J = 12.7 Hz, 2H), 3.35 (br s, 1H), 2.97-3.09 (m, 2H), 2.20 (br d, J = 12.2 Hz, 2H), 1.97- 2.13 (m, 2H), 1.30 (s, 6H) |
| 7 | 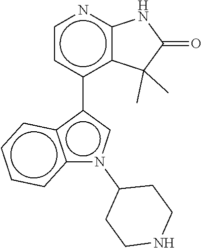 | indole | 361.1 | 1H NMR (DMSO-d6, 500 MHz): δ (ppm) 11.09 (s, 1H), 8.55-9.02 (m, 2H), 8.11 (d, J = 5.4 Hz, 1H), 7.71 (d, J = 8.3 Hz, 1H), 7.51 (s, 1H), 7.35 (d, J = 7.8 Hz, 1H), 7.26 (td, J = 7.6, 1.0 Hz, 1H), 7.10 (td, J = 7.3, 0.5 Hz, 1H), 6.92 (d, J = 5.4 Hz, 1H), 4.85 (tt, J = 11.5, 4.7 Hz, 1H), 3.48 (br d, J = 13.4 Hz, 3H), 3.10-3.23 (m, 2H), 2.17-2.29 (m, 4H), 1.13 (s, 6H) |
| 8 | 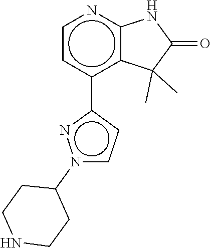 | pyrazole | 312.1 | 1H NMR (DMSO-d6, 500 MHz): δ (ppm) 11.06 (s, 1H), 8.62-9.21 (m, 2H), 8.08 (d, J = 5.6 Hz, 1H), 7.93 (d, J = 2.4 Hz, 1H), 7.25 (d, J = 5.6 Hz, 1H), 6.86 (d, J = 2.4 Hz, 1H), 4.59 (tt, J = 10.3, 5.0 Hz, 1H), 4.25 (br s, 1H), 3.43 (br d, J = 13.0 Hz, 2H), 3.02-3.14 (m, 2H), 2.13-2.29 (m, 4H), 1.48 (s, 6H) |
| 9 | 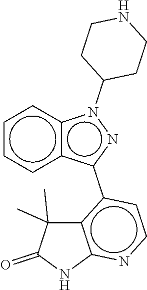 | Indazole 1 | 362 | 1H NMR (DMSO-d6, 500 MHz): δ (ppm) 11.14 (s, 1H), 8.61-9.12 (m, 2H), 8.23 (d, J = 5.4 Hz, 1H), 7.87- 7.89 (m, 1H), 7.85-7.86 (m, 1H), 7.47-7.61 (m, 1H), 7.34 (d, J = 5.4 Hz, 1H), 7.26-7.31 (m, 1H), 5.13 (tt, J = 11.5, 3.8 Hz, 1H), 4.64 (s, 1H), 3.05-3.65 (m, 4H), 2.12-2.43 (m, 4H), 1.38 (s, 6H) |
| 10 | 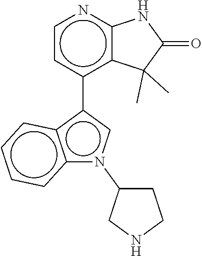 | indole | 347.1 | 1H NMR (DMSO-d6, 500 MHz): δ (ppm) 11.11 (s, 1H), 9.28-9.87 (m, 2H), 8.10 (d, J = 5.4 Hz, 1H), 7.78 (s, 1H), 7.65 (d, J = 8.3 Hz, 1H), 7.31 (d, J = 7.8 Hz, 1H), 7.27 (td, J = 7.7, 1.0 Hz, 1H), 7.08-7.13 (m, 1H), 6.88 (d, J = 5.4 Hz, 1H), 5.48 (quin, J = 7.3 Hz, 1H), 4.38 (br s, 1H), 3.74-3.88 (m, 1H), 3.40-3.55 (m, 2H), 3.27- 3.38 (m, 1H), 2.52-2.61 (m, 1H), 2.32 (dq, J = 13.2, 7.8 Hz, 1H), 1.12 (d, J = 4.6 Hz, 6H) |
| 11 | 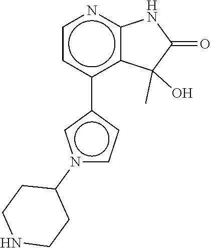 | pyrrole | 313 | 1H NMR (DMSO-d6, 500 MHz): δ (ppm) 10.96 (br s, 1H), 8.62-9.06 (m, 2H), 7.96 (d, J = 5.9 Hz, 1H), 7.86 (t, J = 1.8 Hz, 1H), 7.15 (d, J = 5.9 Hz, 1H), 6.94 (t, J = 2.4 Hz, 1H), 6.81 (dd, J = 2.6, 1.8 Hz, 1H), 4.31 (ddt, J = 11.5, 7.7, 4.0 Hz, 2H), 4.05 (br s, 1H), 3.41 (br d, J = 12.5 Hz, 2H), 2.98-3.13 (m, 2H), 2.19 (br d, J = 12.0 Hz, 2H), 1.98-2.13 (m, 2H), 1.38 (s, 3H) |
| 12 | 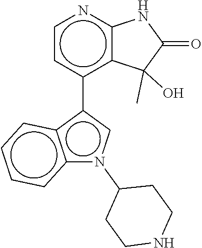 | indole | 363 | 1H NMR (DMSO-d6, 500 MHz): δ (ppm) 10.98 (s, 1H), 8.62-9.09 (m, 2H), 8.47 (s, 1H), 8.13 (d, J = 5.6 Hz, 1H), 7.87 (d, J = 8.1 Hz, 1H), 7.75 (d, J = 8.3 Hz, 1H), 7.35 (d, J = 5.6 Hz, 1H), 7.26-7.31 (m, 1H), 7.18-7.22 (m, 1H), 6.49 (br s, 1H), 4.76-4.93 (m, 1H), 4.17 (br s, 1H), 3.40-3.54 (m, 2H), 3.12-3.27 (m, 2H), 2.17- 2.28 (m, 3H), 2.07-2.17 (m, 1H), 1.24 (s, 3H) |
| 13 | 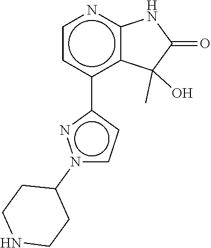 | pyrazole | 314 | 1H NMR (DMSO-d6, 600 MHz): δ (ppm) 11.03 (s, 1H), 8.63-9.12 (m, 2H), 8.11 (d, J = 5.6 Hz, 1H), 7.98 (d, J = 2.3 Hz, 1H), 7.39 (d, J = 5.6 Hz, 1H), 7.18 (d, J = 2.5 Hz, 1H), 6.30 (br s, 1H), 4.63 (tt, J = 11.1, 4.2 Hz, 1H), 3.99 (br s, 1H), 3.39-3.49 (m, 2H), 3.04-3.12 (m, 2H), 2.09-2.31 (m, 4H), 1.43 (s, 3H) |
| 14 | 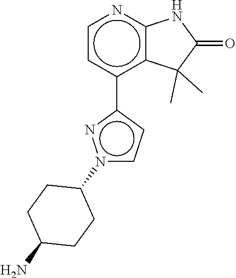 | pyrazole | 326 | 1H NMR (DMSO-d6, 500 MHz): δ (ppm) 11.05 (s, 1H), 8.07 (br d, J = 5.4 Hz, 1H), 8.03 (br s, 3H), 7.91 (d, J = 2.4 Hz, 1H), 7.23 (d, J = 5.6 Hz, 1H), 6.81 (d, J = 2.2 Hz, 1H), 5.00 (br s, 1H), 4.24-4.32 (m, 1H), 3.05 (s, 1H), 2.13-2.22 (m, 2H), 2.10 (br d, J = 12.0 Hz, 2H), 1.81-1.94 (m, J = 12.7, 12.7, 12.7, 2.8 Hz, 2H), 1.51- 1.63 (m, 2H), 1.48 (s, 6H) |
| 15 | 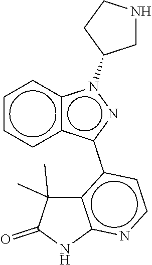 | Indazole 2 | 348 | 1H NMR (600 MHz, DMSO-d6) δ ppm 11.15 (s, 1H) 8.97-9.68 (m, 2H) 8.23 (d, J = 5.43 Hz, 1H) 7.84- 7.89 (m, 1H) 7.77-7.82 (m, 1H) 7.50-7.61 (m, 1H) 7.28-7.34 (m, 1H) 7.26 (d, J = 5.43 Hz, 1H) 5.53- 5.93 (m, 1H) 3.54-3.93 (m, 2H) 3.40-3.53 (m, 2H) 2.55-2.64 (m, 1H) 2.34-2.46 (m, 1H) 1.32 (s, 6H) |
| 16 | 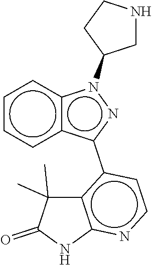 | Indazole 2 | 348 | 1H NMR (DMSO-d6, 500 MHz): δ (ppm) 11.14 (br s, 1H), 8.23 (d, J = 5.4 Hz, 1H), 7.87 (d, J = 8.6 Hz, 1H), 7.82 (d, J = 8.3 Hz, 1H), 7.53 (t, J = 7.7 Hz, 1H), 7.28 (q, J = 4.8 Hz, 2H), 5.60 (s, 1H), 3.51-3.72 (m, 2H), 3.37 (br s, 4H), 2.44-2.48 (m, 1H), 2.33 (br d, J = 5.9 Hz, 1H), 1.91 (s, 1H), 1.36 (s, 3H), 1.31 (s, 3H) |
| 17 | 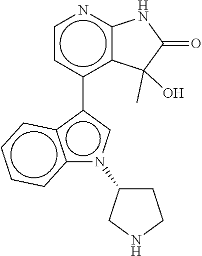 | indole | 349 | 1H NMR (DMSO-d6, 500 MHz): δ (ppm) 10.97 (s, 1H), 9.49 (br s, 2H), 8.45 (s, 1H), 8.14 (dd, J = 5.6, 2.0 Hz, 1H), 7.85 (dd, J = 7.9, 4.0 Hz, 1H), 7.69 (d, J = 8.1 Hz, 1H), 7.28- 7.34 (m, 2H), 7.19-7.25 (m, 1H), 5.42 (dt, J = 19.1, 7.2 Hz, 1H), 3.99 (br s, 2H), 3.18-3.60 (m, 4H), 2.53- 2.65 (m, 1H), 2.27-2.42 (m, 1H), 1.27 (s, 1,5H), 1.24 (s, 1,5H) |
| 18 | 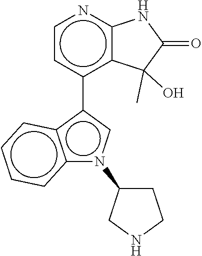 | indole | 349 | 1H NMR (DMSO-d6, 500 MHz): Shift (ppm) 10.97 (s, 1H), 9.49 (br s, 2H), 8.45 (s, 1H), 8.14 (dd, J = 5.6, 2.0 Hz, 1H), 7.85 (dd, J = 7.9, 4.3 Hz, 1H), 7.69 (d, J = 8.3 Hz, 1H), 7.28-7.33 (m, 2H), 7.22 (t, J = 7.5 Hz, 1H), 5.59- 7.00 (br s, 1H), 5.37-5.47 (m, 1H), 3.75-3.83 (m, 1H), 3.69 (br dd, J = 17.0, 5.3 Hz, 1H), 3.52 (dq, J = 11.9, 5.7 Hz, 1H), 3.28-3.47 (m, 1H), 3.24-3.35 (m, 1H), 2.54-2.64 (m, 1H), 2.31-2.42 (m, 1H), 1.25 (d, J = 13.7 Hz, 3H) |
| 19 | 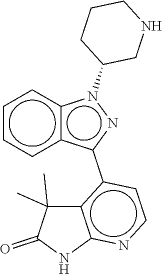 | Indazole 1 | 362 | 1H NMR (DMSO-d6, 500 MHz): δ (ppm) 11.16 (s, 1H), 9.18-9.50 (m, 2H), 8.23 (d, J = 5.4 Hz, 1H), 7.83 (dd, J = 17.5, 8.4 Hz, 2H), 7.50-7.66 (m, 1H), 7.23-7.36 (m, 2H), 5.22 (tt, J = 11.3, 3.9 Hz, 1H), 3.62 (br d, J = 11.5 Hz, 1H), 3.20-3.45 (m, 2H), 2.95-3.15 (m, 1H), 2.12-2.34 (m, 2H), 2.03 (br dd, J = 6.7, 4.0 Hz, 2H), 1.28-1.45 (m, 6H) |
| 20 | 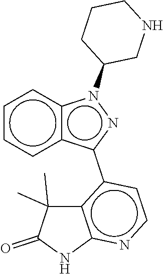 | Indazole 1 | 362 | 1H NMR (600 MHz, DMSO-d6) δ ppm 1.23-1.43 (m, 6H), 1.93- 2.09 (m, 2H), 2.13-2.30 (m, 2H), 2.97-3.10 (m, 1H), 3.23-3.35 (m, 1H), 3.36-3.41 (m, 1H), 3.59- 3.62 (m, 1H), 5.12-5.30 (m, 1H), 7.25-7.35 (m, 2H), 7.57 (m, J = 1.00 Hz, 1H), 7.76-7.90 (m, 2H), 8.23 (d, J = 5.43 Hz, 1H), 9.17-9.45 (m, 2H), 11.15 (s, 1H) |
| 21 | 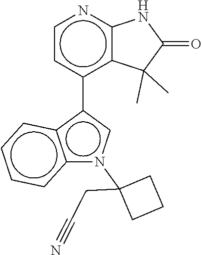 | Specific Indole 1 | 371 | 1H NMR (DMSO-d6, 600 MHz): δ (ppm) 11.04 (s, 1H), 8.09 (d, J = 5.4 Hz, 1H), 7.43 (s, 1H), 7.42 (d, J = 8.4 Hz, 1H), 7.34 (d, J = 7.9 Hz, 1H), 7.21 (td, J = 7.7, 1.0 Hz, 1H), 7.07- 7.12 (m, 1H), 6.89 (d, J = 5.3 Hz, 1H), 3.46 (s, 2H), 2.72-2.81 (m, 2H), 2.58-2.66 (m, 2H), 2.13-2.26 (m, 1H), 1.92-2.03 (m, 1H), 1.15 (s, 6H) |
| 22 | 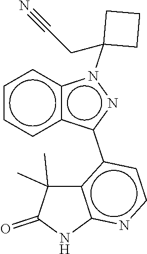 | Indazole 3 | 372 | 1H NMR (DMSO-d6, 600 MHz): δ (ppm) 11.11 (s, 1H), 8.22 (d, J = 5.4 Hz, 1H), 7.87 (d, J = 8.4 Hz, 1H), 7.68 (dt, J = 8.5, 0.9 Hz, 1H), 7.49 (ddd, J = 8.4, 7.0, 1.0 Hz, 1H), 7.32 (d, J = 5.4 Hz, 1H), 7.30 (ddd, J = 8.1, 7.0, 0.6 Hz, 1H), 3.49 (s, 2H), 2.90-2.98 (m, 2H), 2.62-2.68 (m, 2H), 2.19-2.25 (m, 1H), 1.95-2.04 (m, 1H), 1.37 (s, 6H) |
| 23 | 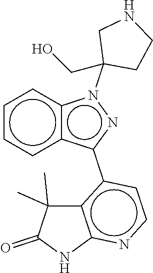 | Indazole 5 | 378 | 1H NMR (600 MHz, DMSO-d6) δ ppm 11.13 (s, 1H), 8.86-9.79 (m, 2H), 8.23 (d, J = 5.43 Hz, 1H), 7.85 (d, J = 9.24 Hz, 2H), 7.46-7.54 (m, 1H), 7.28-7.32 (m, 1H), 7.25-7.28 (m, 1H), 5.39-5.57 (m, 1H), 4.10- 4.29 (m, 1H), 3.79-4.05 (m, 3H), 3.35-3.52 (m, 2H), 2.70-3.06 (m, 2H), 1.15-1.47 (m, 6H) |
| 24 | 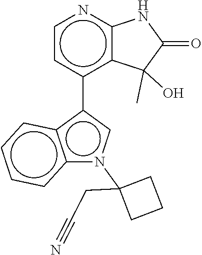 | Specific Indole 1 | 373 | 1H NMR (DMSO-d6, 500 MHz): δ (ppm) 10.98 (s, 1H), 8.44 (s, 1H), 8.13 (d, J = 5.6 Hz, 1H), 7.83-7.94 (m, 1H), 7.45 (d, J = 7.6 Hz, 1H), 7.36 (d, J = 5.9 Hz, 1H), 7.14-7.29 (m, 2H), 3.85-5.57 (m, 1H), 3.31- 3.54 (m, 2H), 2.57-2.85 (m, 4H), 2.13-2.30 (m, 1H), 1.88-2.06 (m, 1H), 1.29 (s, 3H) |
| 25 | 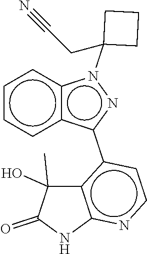 | Indazole 3 | 374.2 | 1H NMR (DMSO-d6, 500 MHz): δ (ppm) 11.09 (br s, 1H), 8.27 (d, J = 5.4 Hz, 1H), 8.09 (d, J = 8.3 Hz, 1H), 7.70 (d, J = 8.6 Hz, 1H), 7.54 (ddd, J = 8.4, 7.2, 1.0 Hz, 1H), 7.51 (d, J = 5.6 Hz, 1H), 7.36 (dd, J = 7.8, 6.8 Hz, 1H), 5.98 (s, 1H), 3.49 (d, J = 2.2 Hz, 2H), 2.79-3.05 (m, 2H), 2.64 (ddd, J = 12.2, 8.9, 2.7 Hz, 2H), 1.93-2.30 (m, 2H), 1.48 (s, 3H) |
| 26 | 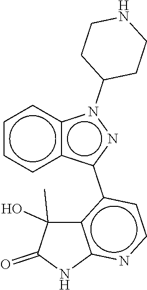 | Indazole 1 | 364 | 1H NMR (DMSO-d6, 500 MHz): δ (ppm) 11.12 (s, 1H), 8.96 (br s, 1H), 8.72 (br s, 1H), 8.27 (d, J = 5.6 Hz, 1H), 8.06 (d, J = 8.3 Hz, 1H), 7.91 (d, J = 8.8 Hz, 1H), 7.59 (ddd, J = 8.3, 7.1, 0.7 Hz, 1H), 7.50 (d, J = 5.6 Hz, 1H), 7.33-7.37 (m, 1H), 5.95 (br s, 1H), 5.18 (tt, J = 11.2, 4.0 Hz, 1H), 3.85 (br s, 1H), 3.47-3.59 (m, 2H), 3.11-3.27 (m, 2H), 2.13-2.40 (m, 4H), 1.46 (s, 3H) |
| 27 | 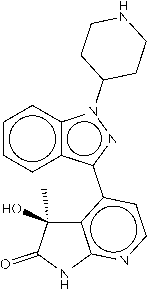 | Indazole 1 (using compound II′ obtained from OHMe isomer 1) | 364 | 1H NMR (DMSO-d6, 600 MHz): δ (ppm) 11.12 (s, 1H), 8.62-9.07 (m, 2H), 8.27 (d, J = 5.4 Hz, 1H), 8.06 (d, J = 8.4 Hz, 1H), 7.91 (d, J = 8.7 Hz, 1H), 7.59 (ddd, J = 8.3, 7.1, 0.9 Hz, 1H), 7.49 (d, J = 5.6 Hz, 1H), 7.33- 7.37 (m, 1H), 5.94 (br s, 1H), 5.18 (tt, J = 11.3, 4.0 Hz, 1H), 4.15 (br s, 1H), 3.49-3.56 (m, 2H), 3.12-3.23 (m, 2H), 2.17-2.37 (m, 4H), 1.46 (s, 3H) |
| 28 | 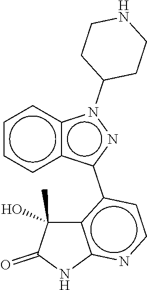 | Indazole 1 (using compound II′ obtained from OHMe isomer 2) | 364 | 1H NMR (DMSO-d6, 500 MHz): δ (ppm) 11.12 (s, 1H), 8.57-9.03 (m, 2H), 8.27 (d, J = 5.4 Hz, 1H), 8.06 (d, J = 8.1 Hz, 1H), 7.91 (d, J = 8.6 Hz, 1H), 7.56-7.62 (m, 1H), 7.50 (d, J = 5.4 Hz, 1H), 7.32-7.38 (m, 1H), 5.95 (br s, 1H), 5.17 (tt, J = 11.2, 3.9 Hz, 1H), 4.36 (br s, 1H), 3.06-3.69 (m, 4H), 2.12-2.42 (m, 4H), 1.46 (s, 3H) |
| 29 | 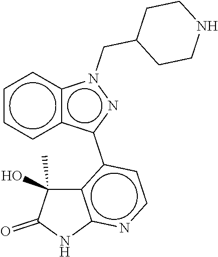 | Indazole 1 (using compound II′ obtained from OHMe isomer 1) | 378 | 1H NMR (600 MHz, DMSO-d6, 300K) δ ppm 11.09 (s, 1H), 8.69- 8.95 (m, 1H), 8.36-8.51 (m, 1H), 8.26 (d, J = 5.4 Hz, 1H), 8.03 (d, J = 8.2 Hz, 1H), 7.89 (d, J = 8.7 Hz, 1H), 7.55 (ddd, J = 8.4, 7.0, 0.9 Hz, 1H), 7.46 (d, J = 5.6 Hz, 1H), 7.32 (ddd, J = 8.0, 7.1, 0.6 Hz, 1H), 4.46- 4.59 (m, 2H), 3.21-3.30 (m, 2H), 2.75-2.90 (m, 2H), 2.27 (dtt, J = 14.8, 7.4, 7.4, 3.5, 3.5 Hz, 1H), 1.73 (br t, J = 13.2 Hz, 2H), 1.41- 1.50 (m, 5H) |
| 30 | 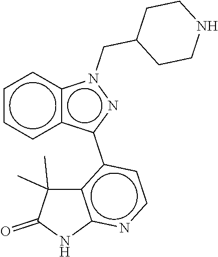 | Indazole 1 | 376 | 1H NMR (600 MHz, DMSO-d6, 300K) δ ppm 11.12 (s, 1H), 8.64- 8.82 (m, 1H), 8.31-8.51 (m, 1H), 8.22 (d, J = 5.4 Hz, 1H), 7.80-7.87 (m, 2H), 7.51 (ddd, J = 8.3, 7.1, 0.9 Hz, 1H), 7.30 (d, J = 5.4 Hz, 1H), 7.25-7.28 (m, 1H), 4.49 (d, J = 7.0 Hz, 2H), 3.27 (br d, J = 12.8 Hz, 2H), 2.75-2.89 (m, 2H), 2.31 (ddd, J = 11.0, 7.4, 3.7 Hz, 1H), 1.72 (br d, J = 12.5 Hz, 2H), 1.41-1.55 (m, 2H), 1.37 (s, 6H) |
| 31 | 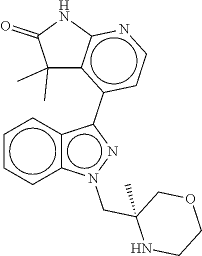 | Indazole 4 | 392.1 | 1H NMR (600 MHz, DMSO-d6) δ 11.10 (br s, 1H), 8.21 (d, J = 5.4 Hz, 1H), 7.83 (d, J = 8.5 Hz, 1H), 7.80 (d, J = 8.2 Hz, 1H), 7.49 (t, J = 7.7 Hz, 1H), 7.29 (d, J = 5.4 Hz, 1H), 7.24 (t, J = 7.6 Hz, 1H), 4.69 (d, J = 14.4 Hz, 1H), 4.53 (d, J = 14.4 Hz, 1H), 3.64 (s, 1H), 3.58 (d, J = 11.0 Hz, 1H), 3.49 (br t, J = 8.0 Hz, 1H), 3.29- 3.27 (m, 1H), 3.10 (br s, 1H), 2.74 (br d, J = 13.6 Hz, 1H), 2.36-2.15 (m, 1H), 1.36 (s, 6H), 0.94 (s, 3H) |
| 32 | 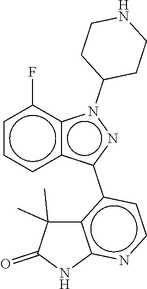 | Indazole 1 | 380 | 1H NMR(DMSO-d6, 500 MHz): δ (ppm) 11.18 (s, 1H), 9.02 (d, J = 10.8 Hz, 1H), 8.76 (d, J = 9.9 Hz, 1H), 8.24 (d, J = 5.4 Hz, 1H), 7.65 (d, J = 8.2 Hz, 1H), 7.38 (dd, J = 12.5, 7.6 Hz, 1H), 7.31-7.21 (m, 2H), 5.13 (td, J = 10.8, 5.2 Hz, 1H), 3.49 (d, J = 12.9 Hz, 2H), 3.23 (q, J = 11.8 Hz, 2H), 2.42-2.29 (m, 4H), 1.35 (s, 6H) |
| 33 | 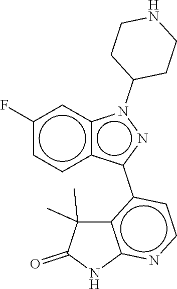 | Indazole 1 | 380 | 1H NMR (DMSO-d6, 500 MHz): δ (ppm) 11.16 (s, 1H), 8.66-9.16 (m, 2H), 8.23 (d, J = 5.4 Hz, 1H), 7.87 (dd, J = 9.0, 5.1 Hz, 1H), 7.78 (dd, J = 9.8, 2.2 Hz, 1H), 7.31 (d, J = 5.4 Hz, 1H), 7.16 (td, J = 9.0, 2.2 Hz, 1H), 5.07 (tt, J = 11.4, 3.9 Hz, 1H), 4.02 (br s, 2H), 3.51 (br d, J = 13.0 Hz, 2H), 3.06-3.21 (m, 2H), 2.28- 2.39 (m, 2H), 2.21 (br d, J = 12.2 Hz, 2H), 1.36 (s, 6H) |
| 34 | 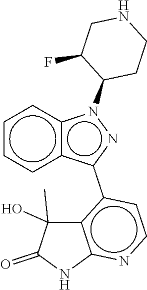 | Indazole 1 | 381.9 | 1H NMR (DMSO-d6, 600 MHz): δ (ppm) 11.13 (d, J = 12.0 Hz, 1H), 8.72-9.75 (m, 2H), 8.28 (dd, J = 5.4, 4.3 Hz, 1H), 8.07-8.20 (m, 1H), 7.96 (dd, J = 8.7, 3.5 Hz, 1H), 7.61-7.67 (m, 1H), 7.49-7.60 (m, 1H), 7.35- 7.44 (m, 1H), 5.90-6.25 (m, 1H), 5.41-5.59 (m, 1H), 5.15-5.39 (m, 1H), 3.40-3.82 (m, 3H), 3.20-3.30 (m, 1H), 2.71-2.95 (m, 1H), 2.27 (br d, J = 13.1 Hz, 1H), 1.30-1.50 (m, 3H) |
| 35 | 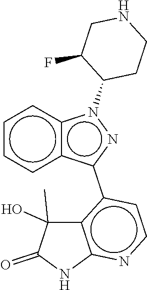 | Indazole 1 | 382 | 1H NMR (DMSO-d6, 500 MHz): δ (ppm) 11.12 (s, 1H), 9.52-9.68 (m, 1H), 9.27 (br d, J = 1.0 Hz, 1H), 8.28 (d, J = 5.6 Hz, 1H), 8.02 (t, J = 8.9 Hz, 1H), 7.87 (d, J = 8.8 Hz, 1H), 7.56-7.66 (m, 1H), 7.43-7.52 (m, 1H), 7.29-7.40 (m, 1H), 5.93 (br s, 1H), 5.38-5.53 (m, 1H), 5.12-5.36 (m, 1H), 3.75-4.07 (m, 1H), 3.48- 3.56 (m, 1H), 3.25-3.36 (m, 1H), 3.14-3.24 (m, 1H), 2.30-2.57 (m, 2H), 1.41-1.56 (m, 3H) |
| 36 | 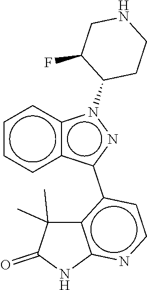 | Indazole 1 | 380 | 1H NMR (500 MHz, DMSO-d6) δ ppm 11.16 (s, 1H); 8.97-10.01 (m, 2H), 8.24 (d, J = 5.38 Hz, 1H); 7.84 (dd, J = 8.31, 5.62 Hz, 2H), 7.50- 7.59 (m, 1H), 7.34 (d, J = 5.62 Hz, 1H), 7.27-7.32 (m, 1H), 5.37-5.48 (m, 1H), 5.19-5.37 (m, 1H); 3.85 (br s, 1H); 3.46-3.54 (m, 1H), 3.24- 3.34 (m, 1H), 3.13-3.24 (m, 1H), 2.29-2.49 (m, 2H), 1.36 (d, J = 13.20 Hz, 6H) |
| 37 | 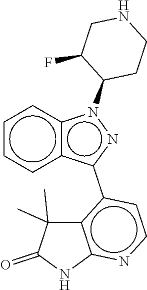 | Indazole 1 | 380 | 1H NMR (DMSO-d6, 600 MHz): δ (ppm) 11.14 (s, 1H), 8.51-9.66 (m, 2H), 8.23 (d, J = 5.4 Hz, 1H), 7.91 (dd, J = 16.2, 8.4 Hz, 2H), 7.56 (ddd, J = 8.4, 7.0, 1.0 Hz, 1H), 7.27- 7.40 (m, 2H), 5.35-5.49 (m, 1H), 5.18-5.35 (m, 1H), 3.54-3.80 (m, 3H), 3.19-3.29 (m, 1H), 2.97 (br dd, J = 13.0, 3.9 Hz, 1H), 2.20-2.32 (m, 1H), 1.42 (s, 3H), 1.32 (s, 3H) |
| 38 | 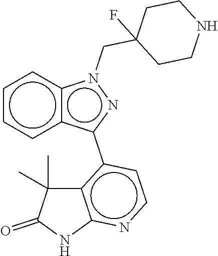 | Indazole 1 | 394 | 1H NMR (500 MHz, DMSO-d6): δ ppm 11.15 (s, 1H), 8.56-9.22 (m, 2H), 8.23 (d, J = 5.38 Hz, 1H), 7.72- 7.90 (m, 2H), 7.44-7.57 (m, 1H), 7.31 (d, J = 5.38 Hz, 1H), 7.25-7.30 (m, 1H), 4.90-4.98 (m, 2H), 3.24- 3.33 (m, 2H), 2.90-3.04 (m, 2H), 1.85-2.22 (m, 4H), 1.37 (s, 6H) |
| 39 | 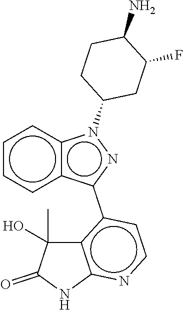 | Indazole 1 (using specific indazole 2 as compound II) | 396.1 | 1H NMR (600 MHz, DMSO-d6): δ ppm 11.10 (d, J = 4.84 Hz, 1H), 8.36- 8.62 (m, 3H), 8.26 (dd, J = 5.43, 1.76 Hz, 1H), 8.01-8.07 (m, 1H), 7.98 (d, J = 8.66 Hz, 1H), 7.53-7.59 (m, 1H), 7.44-7.51 (m, 1H), 7.30- 7.38 (m, 1H), 5.31-6.78 (m, 1H), 4.99-5.09 (m, 1H), 4.83-4.98 (m, 1H), 3.42-3.51 (m, 1H), 2.56- 2.70 (m, 1H), 1.88-2.35 (m, 4H), 1.60-1.79 (m, 1H), 1.45 (m, J = 14.20 Hz, 3H) |
| 40 | 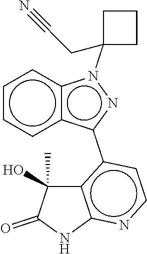 | Indazole 3 (using compound II′ obtained from OHMe isomer 1) | 374.2 | 1H NMR (DMSO-d6, 600 MHz): δ (ppm) 11.09 (s, 1H), 8.27 (d, J = 5.6 Hz, 1H), 8.09 (d, J = 8.2 Hz, 1H), 7.70 (d, J = 8.5 Hz, 1H), 7.52-7.56 (m, 1H), 7.50 (d, J = 5.4 Hz, 1H), 7.36 (ddd, J = 8.1, 7.0, 0.7 Hz, 1H), 5.98 (s, 1H), 3.41-3.56 (m, 2H), 2.84-3.05 (m, 2H), 2.61-2.69 (m, 2H), 2.17-2.30 (m, 1H), 1.96-2.05 (m, 1H), 1.48 (s, 3H) |
| 41 | 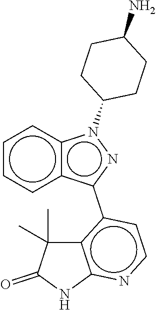 | Indazole 1 (using specific indazole 2 as compound II) | 376 | 1H NMR (600 MHz, DMSO-d6): δ ppm 11.11 (s, 1H), 8.22 (d, J = 5.43 Hz, 1H), 7.98-8.10 (m, 3H), 7.91 (d, J = 8.66 Hz, 1H), 7.81-7.86 (m, 1H), 7.44-7.53 (m, 1H), 7.33 (d, J = 5.43 Hz, 1H), 7.22-7.28 (m, 1H), 4.71-4.85 (m, 1H), 3.11-3.30 (m, 1H), 2.10-2.19 (m, 4H), 1.97- 2.08 (m, 2H), 1.61-1.75 (m, 2H), 1.37 (s, 6H) |
| 42 | 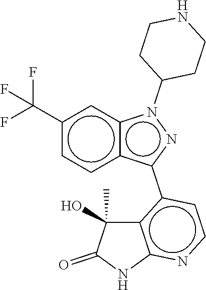 | Indazole 1 (using compound II′ obtained from OHMe isomer 1) | 432.2 | 1H NMR (DMSO-d6, 600 MHz): δ (ppm) 11.13 (s, 1H), 8.62-9.09 (m, 2H), 8.43 (s, 1H), 8.28 (d, J = 5.4 Hz, 1H), 8.19 (d, J = 8.7 Hz, 1H), 7.58 (dd, J = 8.7, 1.3 Hz, 1H), 7.42 (d, J = 5.4 Hz, 1H), 5.83 (br s, 1H), 5.27- 5.37 (m, 1H), 4.32 (br s, 1H), 3.53 (br d, J = 12.3 Hz, 2H), 3.10-3.20 (m, 2H), 2.17-2.37 (m, 4H), 1.50 (s, 3H) |
| 43 | 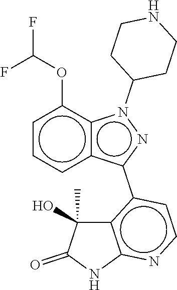 | indazole 2 (using compound II′ obtained from OHMe isomer 1 and specific Indazole 1 as compound II) | 430 | 1H NMR (500 MHz, DMSO-d6) protonated form Shift 11.11 (s, 1H), 9.01 (br s, 1H), 8.73 (s, 1H), 8.71 (br s, 1H), 8.27 (d, J = 5.62 Hz, 1H), 7.80 (dd, J = 0.98, 7.83 Hz, 1H), 7.47 (s, 1H), 7.28-7.37 (m, 2H), 7.27-7.64 (m, 1H), 5.38-6.19 (m, 1H), 5.20- 5.29 (m, 1H), 3.45-3.58 (m, 2H), 3.11-3.20 (m, 2H), 2.25-2.38 (m, 4H), 1.51 (s, 3H) |
| 44 | 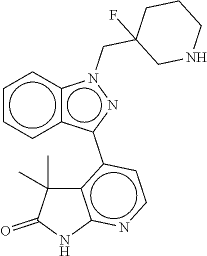 | Indazole 1 | 394 | 1H NMR (500 MHz, DMSO-d6) δ ppm 11.15 (s, 1H), 8.40-9.59 (m, 2H), 8.24 (d, J = 5.38 Hz, 1H), 7.84 (m, J = 8.30, 4.90 Hz, 2H), 7.48- 7.62 (m, 1H), 7.31 (d, J = 5.38 Hz, 1H), 7.28 (d, J = 7.09 Hz, 1H), 4.73- 5.02 (m, 2H), 3.28-3.42 (m, 2H), 3.21 (m, J = 12.00 Hz, 1H), 2.76- 2.99 (m, 1H), 1.83-2.06 (m, 2H), 1.68-1.82 (m, 2H), 1.29-1.45 (m, 6H) |
| 45 | 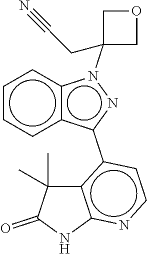 | Indazole 3 | 374 | 1H NMR (600 MHz, DMSO-d6): δ ppm 11.02 (s, 1H), 8.24 (d, J = 5.28 Hz, 1H), 7.88 (d, J = 8.36 Hz, 1H), 7.58-7.67 (m, 1H), 7.50-7.56 (m, 1H), 7.31-7.39 (m, 1H), 7.28- 7.31 (m, 1H), 4.90-5.41 (m, 4H), 3.72 (s, 2H), 1.33 (s, 6H) |
| 46 | 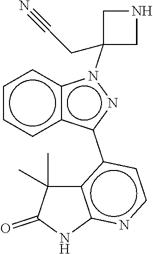 | Indazole 3 | 373 | 1H NMR (500 MHz, DMSO-d6): Shift 11.18 (s, 1H), 9.74 (br s, 1H), 9.34 (br s, 1H), 8.25 (d, J = 5.38 Hz, 1H), 7.90 (d, J = 8.07 Hz, 1H), 7.77 (d, J = 8.56 Hz, 1H), 7.59 (t, J = 7.65 Hz, 1H), 7.38 (t, J = 7.48 Hz, 1H), 7.28 (d, J = 5.38 Hz, 1H), 4.81-4.88 (m, 2H), 4.61-4.67 (m, 2H), 3.79-3.88 (m, 2H), 1.35 (s, 6H) |
| 47 | 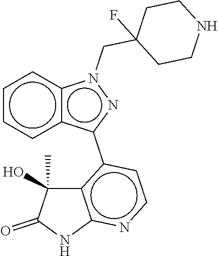 | Indazole 1 (using compound II′ obtained from OHMe isomer 1) | 396 | 1H NMR (500 MHz, DMSO-d6) δ ppm 11.09 (s, 1H), 8.57-9.23 (m, 2H), 8.27 (d, J = 5.38 Hz, 1H), 7.95- 8.11 (m, 1H), 7.78-7.90 (m, 1H), 7.52-7.60 (m, 1H), 7.46 (d, J = 5.38 Hz, 1H), 7.30-7.36 (m, 1H), 5.89 (s, 1H), 4.85-5.10 (m, 2H), 3.21- 3.36 (m, 2H), 2.82-3.11 (m, 2H), 1.82-2.22 (m, 4H), 1.52 (s, 3H) |
| 48 | 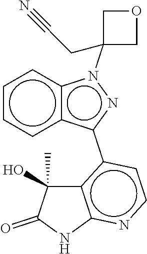 | Indazole 3 (using compound II′ obtained from OHMe isomer 1) | 376 | 1H NMR (600 MHz, DMSO-d6): δ ppm 13.85 (br s, 1H), 8.30 (d, J = 5.43 Hz, 1H), 8.06 (d, J = 8.36 Hz, 1H), 7.67-7.75 (m, 1H), 7.59- 7.66 (m, 1H), 7.44-7.54 (m, 1H), 7.21-7.34 (m, 1H), 6.60 (br s, 1H), 5.06-5.30 (m, 2H), 4.60-4.75 (m, 2H), 3.48-3.51 (m, 2H), 1.50 (s, 3H) |
| 49 | 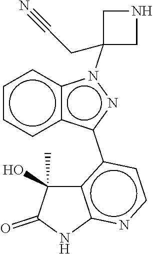 | Indazole 3 (using compound II′ obtained from OHMe isomer 1) | 375 | 1H NMR (DMSO-d6, 500 MHz): δ (ppm) 11.10 (s, 1H), 9.94 (br s, 1H), 9.44 (br s, 1H), 8.27 (d, J = 5.6 Hz, 1H), 8.03 (d, J = 8.3 Hz, 1H), 7.76 (d, J = 8.8 Hz, 1H), 7.58 (ddd, J = 8.4, 7.2, 0.9 Hz, 1H), 7.38 (d, J = 5.6 Hz, 1H), 7.35-7.41 (m, 1H), 5.17-6.26 (m, 1H), 4.79-4.95 (m, 2H), 4.56- 4.67 (m, 2H), 4.28-4.34 (m, 1H), 3.83 (d, J = 3.4 Hz, 2H), 1.51 (s, 3H) |
| 50 | 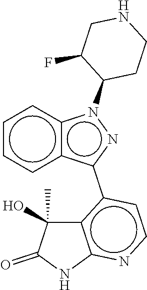 | Indazole 1 (using compound II′ obtained from OHMe isomer 1) | 382 | 1H NMR (600 MHz, DMSO-d6) δ ppm 11.12 (s, 1H), 8.74-9.59 (m, 2H), 8.28 (m, J = 5.40, 4.30 Hz, 1H), 8.06-8.19 (m, 1H), 7.90-7.98 (m, 1H), 7.61-7.67 (m, 1H), 7.50- 7.60 (m, 1H), 7.40 (d, J = 7.63 Hz, 1H), 5.85-6.31 (m, 1H), 5.40-5.59 (m, 1H), 5.22-5.38 (m, 1H), 3.70- 3.85 (m, 1H), 3.48-3.66 (m, 3H), 3.17-3.28 (m, 1H), 2.76-2.91 (m, 1H), 2.22-2.33 (m, 1H), 1.42 (s, 3H) |
| 51 | 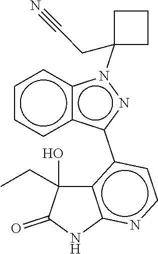 | Indazole 1 (using specific indazole 3 as compound III) | 388.1 | 1H NMR (500 MHz, DMSO-d6) δ ppm 11.13 (br s, 1H), 8.28 (br d, J = 5.14 Hz, 1H), 8.01-8.14 (m, 1H), 7.66-7.76 (m, 1H), 7.42-7.60 (m, 2H), 7.31-7.40 (m, 1H), 6.01 (s, 1H), 3.42-3.57 (m, 2H), 2.56-3.06 (m, 4H), 1.79-2.30 (m, 4H), 0.38- 0.58 (m, 3H) |
| 52 | 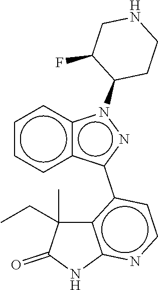 | Indazole 1 | 394 | 1H NMR (600 MHz, DMSO-d6): δ ppm 11.15 (s, 1H), 8.60-9.67 (m, 2H), 8.24 (d, J = 5.43 Hz, 1H), 7.89- 7.94 (m, 1H), 7.84-7.89 (m, 1H), 7.56 (t, J = 7.70 Hz, 1H), 7.27-7.39 (m, 2H), 5.34-5.47 (m, 1H), 5.17- 5.34 (m, 1H), 3.58-3.79 (m, 2H), 3.49-3.55 (m, 1H), 3.19-3.25 (m, 1H), 2.86-3.03 (m, 1H), 2.21- 2.31 (m, 1H), 1.57-2.15 (m, 2H), 1.32-1.50 (m, 3H), 0.28-0.44 (m, 3H) |
| 53 | 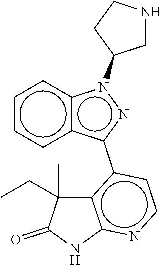 | Indazole 2 | 362 | 1H NMR (DMSO-d6, 500 MHz): δ (ppm) 11.20 (s, 1H), 9.32-9.71 (m, 2H), 8.24 (dd, J = 5.4, 1.5 Hz, 1H), 7.87 (d, J = 8.6 Hz, 1H), 7.75-7.80 (m, 1H), 7.56 (ddd, J = 8.4, 7.0, 1.0 Hz, 1H), 7.27-7.33 (m, 1H), 7.25 (dd, J = 5.5, 1.3 Hz, 1H), 5.73 (quin, J = 6.5 Hz, 1H), 3.80-3.93 (m, 1H), 3.40-3.60 (m, 3H), 2.53-2.63 (m, 1H), 2.33-2.47 (m, 1H), 1.62- 1.90 (m, 2H), 1.26-1.43 (m, 3H), 0.31-0.49 (m, 3H) |
| 54 | 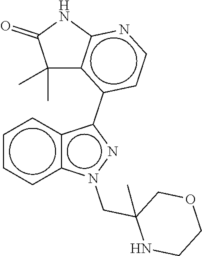 | Indazole 4 | 392.1 | 1H NMR (DMSO-d6, 600 MHz): δ (ppm) 11.10 (s, 1H), 8.21 (d, J = 5.4 Hz, 1H), 7.83 (d, J = 8.7 Hz, 1H), 7.80 (d, J = 8.2 Hz, 1H), 7.49 (ddd, J = 8.3, 7.0, 1.0 Hz, 1H), 7.29 (d, J = 5.4 Hz, 1H), 7.22-7.26 (m, 1H), 4.69 (d, J = 14.5 Hz, 1H), 4.53 (d, J = 14.5 Hz, 1H), 3.65 (dt, J = 10.8, 3.8 Hz, 1H), 3.58 (d, J = 11.2 Hz, 1H), 3.49 (ddd, J = 10.8, 8.3, 2.9 Hz, 1H), 3.28-3.30 (m, 1H), 3.10 (ddd, J = 12.3, 8.6, 3.2 Hz, 1H), 2.71-2.78 (m, 1H), 2.33 (br s, 1H), 1.36 (d, J = 1.6 Hz, 6H), 0.94 (s, 3H) |
| 55 | 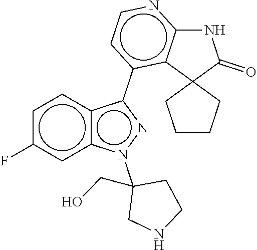 | Indazole 5 | 422.1 | 1H NMR (DMSO-d6, 500 MHz): δ (ppm) 11.02 (s, 1H), 9.18-9.66 (m, 2H), 8.19 (d, J = 5.4 Hz, 1H), 7.65- 7.84 (m, 2H), 7.17 (td, J = 9.0, 2.1 Hz, 1H), 7.11 (d, J = 5.4 Hz, 1H), 4.10 (dt, J = 12.0, 6.0 Hz, 2H), 3.80- 3.97 (m, 3H), 3.40-3.52 (m, 1H), 3.23-3.34 (m, 1H), 2.66-2.93 (m, 2H), 2.10-2.28 (m, 2H), 1.77-1.90 (m, 4H), 1.60 (br d, J = 2.7 Hz, 2H) |
| 56 | 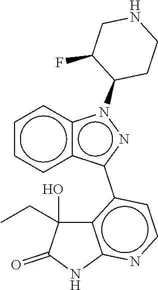 | Indazole 1 | 396.1 | 1H NMR (600 MHz, DMSO-d6) δ ppm 11.15 (m, J = 8.40 Hz, 1H), 8.70- 9.62 (m, 2H), 8.29 (dd, J = 5.43, 3.81 Hz, 1H), 8.06-8.20 (m, 1H), 7.91-7.98 (m, 1H), 7.61-7.66 (m, 1H), 7.49-7.60 (m, 1H), 7.36- 7.43 (m, 1H), 5.72-6.51 (m, 1H), 5.39-5.61 (m, 1H), 5.20-5.38 (m, 1H), 3.73-3.85 (m, 1H), 3.59- 3.66 (m, 2H), 3.18-3.32 (m, 1H), 2.71-2.89 (m, 1H), 2.16-2.29 (m, 1H), 1.78-1.99 (m, 2H), 0.37- 0.49 (m, 3H) |
| 57 | 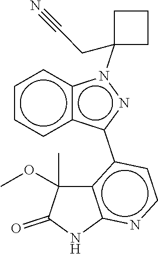 | Indazole 3 | 388.1 | 1H NMR (DMSO-d6, 500 MHz): δ (ppm) 11.30 (s, 1H), 8.30 (d, J = 5.4 Hz, 1H), 7.89 (d, J = 8.3 Hz, 1H), 7.69 (d, J = 8.6 Hz, 1H), 7.48 (ddd, J = 8.4, 7.0, 1.0 Hz, 1H), 7.39 (d, J = 5.4 Hz, 1H), 7.27-7.32 (m, 1H), 3.43 (d, J = 2.7 Hz, 2H), 2.90-3.05 (m, 2H), 2.80 (s, 3H), 2.62 (tt, J = 8.0, 3.9 Hz, 2H), 2.14-2.28 (m, 1H), 1.94- 2.03 (m, 1H), 1.59 (s, 3H) |
| 58 | 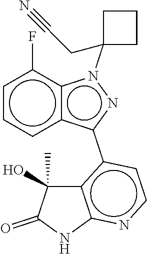 | Indazole 3 (using compound II′ obtained from OHMe isomer 1) | 392 | 1H NMR (CHLOROFORM-d, 500 MHz): δ (ppm) 8.34 (d, J = 5.6 Hz, 1H), 8.18 (br s, 1H), 7.91 (d, J = 8.1 Hz, 1H), 7.51 (d, J = 5.4 Hz, 1H), 7.30-7.36 (m, 1H), 7.24-7.30 (m, 1H), 6.12 (s, 1H), 3.13-3.23 (m, 2H), 2.95-3.13 (m, 2H), 2.67-2.79 (m, 2H), 2.07-2.24 (m, 2H), 1.58 (s, 3H) |
| 59 | 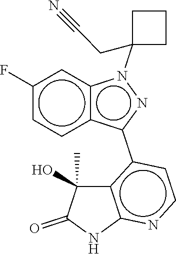 | Indazole 3 (using compound II′ obtained from OHMe isomer 1) | 392 | 1H NMR (600 MHz, DMSO-d6): δ ppm 11.09 (s, 1H), 8.26 (d, J = 5.43 Hz, 1H), 8.05-8.11 (m, 1H), 7.50- 7.57 (m, 1H), 7.44 (d, J = 5.58 Hz, 1H), 7.19-7.25 (m, 1H), 5.88 (s, 1H), 3.51 (s, 2H), 2.81-3.01 (m, 2H), 2.59-2.67 (m, 2H), 1.93-2.27 (m, 2H), 1.47 (s, 3H) |
| 60 | 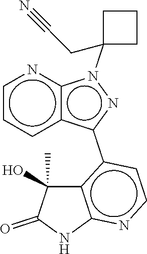 | Indazole 3 (using compound II′ obtained from OHMe isomer 1) | 375.1 | 1H NMR (600 MHz, DMSO-d6) δ 11.11 (s, 1H), 8.68 (dd, J = 1.47, 4.40 Hz, 1H), 8.55 (dd, J = 1.47, 8.22 Hz, 1H), 8.27 (d, J = 5.43 Hz, 1H), 7.46 (d, J = 5.58 Hz, 1H), 7.42 (dd, J = 4.40, 8.22 Hz, 1H), 5.88 (s, 1H), 3.56-3.65 (m, 2H), 2.97-3.15 (m, 2H), 2.53- 2.62 (m, 2H), 2.15-2.23 (m, 1H), 1.98-2.04 (m, 1H), 1.50 (s, 3H) |
| 61 | 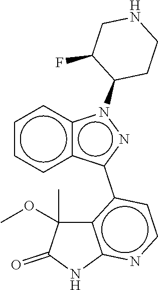 | Indazole 1 | 396 | 1H NMR (DMSO-d6, 500 MHz): δ (ppm) 11.32 (s, 1H), 9.28-9.59 (m, 1H), 8.76-9.10 (m, 1H), 8.29-8.35 (m, 1H), 7.86 (d, J = 8.1 Hz, 2H), 7.51-7.61 (m, 1H), 7.26-7.45 (m, 2H), 5.02-5.57 (m, 2H), 3.33-3.81 (m, 3H), 3.17-3.32 (m, 1H), 2.89- 3.03 (m, 1H), 2.71-2.87 (m, 3H), 2.15-2.31 (m, 1H), 1.44-1.75 (m, 3H) |
| 62 | 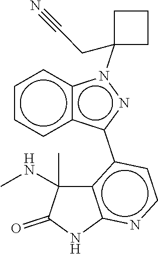 | Indazole 1 (using specific indazole 3 as compound III) | 387.1 | 1H NMR (DMSO-d6, 600 MHz): δ (ppm) 11.10 (s, 1H), 8.25 (d, J = 5.6 Hz, 1H), 8.07 (d, J = 8.2 Hz, 1H), 7.68 (d, J = 8.5 Hz, 1H), 7.51 (d, J = 5.4 Hz, 1H), 7.48-7.54 (m, 1H), 7.34 (ddd, J = 8.0, 7.1, 0.6 Hz, 1H), 3.45- 3.57 (m, 2H), 2.99 (br s, 1H), 2.86- 2.96 (m, 2H), 2.64 (ddd, J = 11.7, 8.1, 3.1 Hz, 2H), 2.17-2.29 (m, 1H), 1.94-2.04 (m, 1H), 1.88 (s, 3H), 1.39 (s, 3H) |
| 63 | 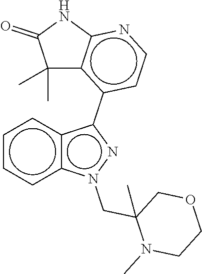 | Indazole 4 | 406 | 1H NMR (DMSO-d6, 500 MHz): δ (ppm) 11.39-11.88 (m, 1H), 11.16 (br s, 1H), 8.23 (dd, J = 5.1, 1.7 Hz, 1H), 7.88-8.11 (m, 1H), 7.82 (br t, J = 9.0 Hz, 1H), 7.16-7.71 (m, 3H), 4.72-5.44 (m, 2H), 3.43 (s, 6H), 2.86-3.04 (m, 3H), 1.15-1.62 (m, 9H) |
| 64 | 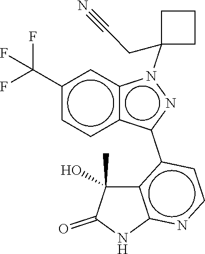 | Indazole 3 (using compound II′ obtained from OHMe isomer 2) | 442.1 | 1H NMR (600 MHz, DMSO-d6) δ ppm 11.10 (s, 1H), 8.28 (d, J = 5.58 Hz, 1H), 8.19-8.24 (m, 1H), 8.01 (s, 1H), 7.55-7.66 (m, 1H), 7.41 (d, J = 5.43 Hz, 1H), 5.77 (s, 1H), 3.54 (s, 2H), 2.69 (m, J = 9.20, 6.10, 3.20 Hz, 4H), 1.93-2.31 (m, 2H) 1.50 (s, 3H) |
| 65 | 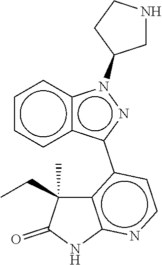 | Indazole 2 (using compound II′ obtained from MeEt isomer 1) | 362.1 | 1H NMR (600 MHz, DMSO-d6) δ ppm 11.12 (s, 1H), 8.18-8.27 (m, 1H), 7.85 (d, J = 8.51 Hz, 1H), 7.79- 7.83 (m, 1H), 7.49 (ddd, J = 8.36, 7.04, 1.03 Hz, 1H), 7.28-7.34 (m, 1H), 7.22-7.27 (m, 1H), 5.33-5.43 (m, 1H), 3.36-3.40 (m, 1H), 3,33- 3.34 (m, 1H), 3.08-3.16 (m, 1H), 3.02-3.06 (m, 1H), 2.94-3.02 (m, 1H), 2.14-2.36 (m, 2H), 1.64- 2.04 (m, 2H), 1.39 (s, 3H), 0.32- 0.44 (m, 3H) |
| 66 | 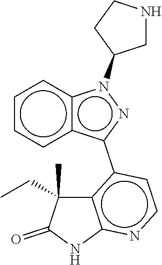 | Indazole 2 (using compound II′ obtained from MeEt isomer 2) | 362.1 | 1H NMR (600 MHz, DMSO-d6): δ ppm 11.22 (s, 1H), 8.23 (d, J = 5.43 Hz, 1H), 7.86 (d, J = 8.51 Hz, 1H), 7.78 (d, J = 8.22 Hz, 1H), 7.51-7.56 (m, 1H), 7.27-7.31 (m, 1H), 7.25- 7.26 (m, 1H), 5.56-5.69 (m, 1H), 3.70-3.77 (m, 1H), 3.42-3.45 (m, 1H), 3.38-3.40 (m, 2H), 3.29- 3.30 (m, 1H), 2.27-2.49 (m, 2H), 1.68-1.94 (m, 2H), 1.30-1.33 (m, 3H), 0.38-0.48 (m, 3H) |
| 67 | 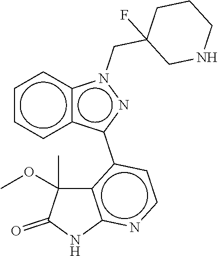 | Indazole 1 | 410.1 | 1H NMR (600 MHz, DMSO-d6, 300K) δ ppm 11.34 (d, J = 4.4 Hz, 1H), 9.26-9.52 (m, 1H), 8.46-8.85 (m, 1H), 8.25-8.35 (m, 1H), 7.72- 7.91 (m, 2H), 7.46-7.60 (m, 1H), 7.32-7.40 (m, 1H), 7.21-7.31 (m, 1H), 4.60-5.12 (m, 2H), 3.12- 3.58 (m, 3H), 2.65-2.91 (m, 4H), 1.55-2.16 (m, 7H) |
| 68 | 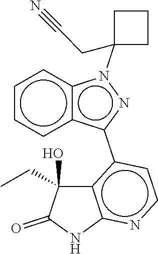 | Indazole 1 (using specific indazole 3 as compound III and compound II′ obtained from EtOH isomer 1) | 388.1 | 1H NMR (600 MHz, DMSO-d6) δ ppm 11.12 (s, 1H), 8.28 (d, J = 5.57 Hz, 1H), 8.07-8.17 (m, 1H), 7.66- 7.75 (m, 1H), 7.52-7.57 (m, 1H), 7.49-7.52 (m, 1H), 7.32-7.39 (m, 1H), 6.00 (s, 1H), 3.39-3.57 (m, 2H), 2.57-3.05 (m, 4H), 2.23 (m, J = 11.00 Hz, 1H), 1.80-2.05 (m, 3H), 0.47 (t, J = 1.00 Hz, 3H) |
| 69 | 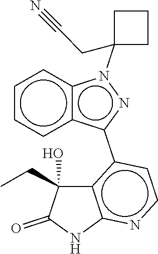 | Indazole 1 (using specific indazole 3 as compound III and compound II′ obtained from EtOH isomer 2) | 388.1 | 1H NMR (500 MHz, DMSO-d6) δ 11.13 (s, 1H), 8.28 (d, J = 5.62 Hz, 1H), 8.11 (d, J = 8.31 Hz, 1H), 7.70 (d, J = 8.56 Hz, 1H), 7.54 (ddd, J = 0.86, 7.15, 8.38 Hz, 1H), 7.51 (d, J = 5.38 Hz, 1H), 7.36 (t, J = 7.54 Hz, 1H), 6.01 (s, 1H), 3.44-3.53 (m, 2H), 2.86-3.00 (m, 2H), 2.63 (ddd, J = 3.55, 6.11, 9.17 Hz, 2H), 2.19-2.29 (m, 1H), 1.96-2.04 (m, 2H), 1.83-1.91 (m, 1H), 0.47 (t, J = 7.46 Hz, 3H) |
| 70 | 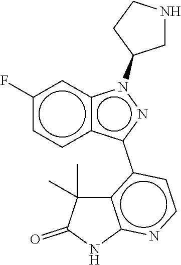 | Indazole 2 | 366.1 | 1H NMR (600 MHz, DMSO-d6) δ 11.17 (s, 1H), 9.55 (br s, 1H), 9.42 (br s, 1H), 8.23 (d, J = 5.43 Hz, 1H), 7.78-7.82 (m, 2H), 7.23 (d, J = 5.43 Hz, 1H), 7.14-7.20 (m, 1H), 5.65- 5.70 (m, 1H), 3.84-3.90 (m, 1H), 3.40-3.58 (m, 3H), 2.52-2.61 (m, 1H), 2.40 (qd, J = 6.58, 12.82 Hz, 1H), 1.23-1.37 (m, 6H) |
| 71 | 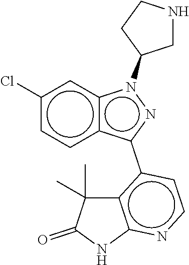 | Indazole 2 | 382.0; 383.9 | 1H NMR (500 MHz, DMSO-d6) δ 11.18 (s, 1H), 9.40 (br s, 1H), 9.34 (br s, 1H), 8.24 (d, J = 5.38 Hz, 1H), 8.11 (d, J = 1.47 Hz, 1H), 7.79 (d, J = 8.80 Hz, 1H), 7.31 (dd, J = 1.83, 8.68 Hz, 1H), 7.23 (d, J = 5.38 Hz, 1H), 5.71-5.77 (m, 1H), 3.70-3.90 (m, 1H), 3.47-3.66 (m, 2H), 3.40- 3.46 (m, 1H), 2.52-2.61 (m, 1H), 2.36-2.43 (m, 1H), 1.17-1.42 (m, 6H) |
| 72 | 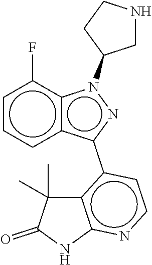 | Indazole 2 | 366 | 1H NMR (DMSO-d6, 600 MHz): δ (ppm) 11.19 (s, 1H), 9.19-9.55 (m, 2H), 8.24 (d, J = 5.4 Hz, 1H), 7.58 (d, J = 8.1 Hz, 1H), 7.41 (dd, J = 12.5, 7.6 Hz, 1H), 7.25 (td, J = 7.9, 4.3 Hz, 1H), 7.21 (d, J = 5.3 Hz, 1H), 5.70- 5.81 (m, 1H), 3.86-3.90 (m, 1H), 3.62 (dq, J = 12.2, 6.1 Hz, 1H), 3.42- 3.52 (m, 2H), 2.51-2.65 (m, 2H), 1.21-1.35 (m, 6H) |
| 73 | 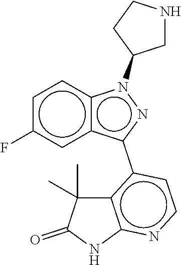 | Indazole 2 | 366.1 | 1H NMR (600 MHz, DMSO-d6) δ ppm 11.16 (s, 1H), 8.99-9.97 (m, 2H), 8.22 (d, J = 5.43 Hz, 1H), 7.91- 7.99 (m, 1H), 7.53-7.60 (m, 1H), 7.41-7.52 (m, 1H), 7.22-7.31 (m, 1H), 5.65-6.16 (m, 1H), 3.81- 3.89 (m, 1H), 3.37-3.58 (m, 3H), 2.54-2.63 (m, 1H), 2.34-2.45 (m, 1H), 1.31 (s, 6H) |
| 74 | 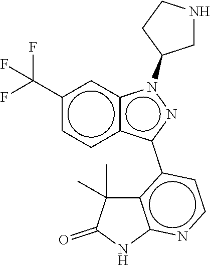 | Indazole 2 | 416.1 | 1H NMR (500 MHz, DMSO-d6, 300K) δ (ppm) = 11.21 (s, 1H), 9.61- 9.31 (m, 2H), 8.45 (s, 1H), 8.26 (d, J = 5.4 Hz, 1H), 8.00 (d, J = 8.6 Hz, 1H), 7.56 (dd, J = 1.1, 8.7 Hz, 1H), 7.26 (d, J = 5.4 Hz, 1H), 6.06-5.79 (m, 1H), 3.98-3.83 (m, 1H), 3.58- 3.40 (m, 3H), 2.68-2.53 (m, 1H), 2.42 (td, J = 6.4, 13.3 Hz, 1H), 1.35 (s, 3H), 1.25 (s, 3H) |
| 75 | 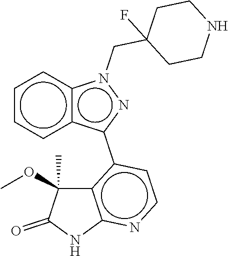 | Indazole 1 (using compound II′ obtained from OHMe isomer 1) | 410.1 | 1H NMR (DMSO-d6, 600 MHz): δ (ppm) 11.32 (s, 1H), 8.55-9.10 (m, 2H), 8.31 (d, J = 5.4 Hz, 1H), 7.83 (d, J = 8.2 Hz, 1H), 7.79 (d, J = 8.5 Hz, 1H), 7.51 (ddd, J = 8.4, 7.0, 0.9 Hz, 1H), 7.36 (d, J = 5.3 Hz, 1H), 7.23-7.30 (m, 1H), 4.80-4.95 (m, 2H), 3.21-3.34 (m, 2H), 2.92-3.04 (m, 2H), 2.74 (s, 3H), 1.86-2.17 (m, 4H), 1.68 (s, 3H) |
| 76 | 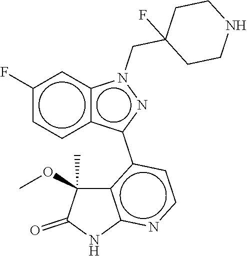 | Indazole 1 (using compound II′ obtained from OHMe isomer 1) | 428.1 | 1H NMR (DMSO-d6, 600 MHz): δ (ppm) 11.34 (s, 1H), 8.53-9.00 (m, 2H), 8.31 (d, J = 5.3 Hz, 1H), 7.85 (dd, J = 8.9, 5.1 Hz, 1H), 7.66 (d, J = 9.8 Hz, 1H), 7.33 (d, J = 5.4 Hz, 1H), 7.15 (td, J = 9.0, 2.2 Hz, 1H), 4.74- 4.98 (m, 2H), 3.22-3.32 (m, 2H), 2.91-3.04 (m, 2H), 2.75 (s, 3H), 1.86-2.17 (m, 4H), 1.65 (s, 3H) |
| 77 | 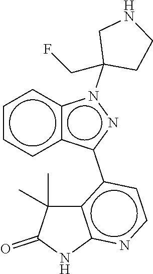 | Indazole 5 | 380.3 | 1H NMR (DMSO-d6, 500 MHz): δ (ppm) 11.11 (br s, 1H), 8.22 (d, J = 5.6 Hz, 1H), 7.85 (d, J = 8.3 Hz, 1H), 7.81 (d, J = 8.6 Hz, 1H), 7.48 (t, J = 7.7 Hz, 1H), 7.31 (d, J = 5.4 Hz, 1H), 7.28 (t, J = 7.3 Hz, 1H), 4.63-4.98 (m, 2H), 3.61 (br s, 2H), 2.81-3.17 (m, 3H), 2.63 (tt, J = 8.5, 4.2 Hz, 1H), 2.41-2.48 (m, 1H), 1.37 (s, 3H), 1.31 (s, 3H) |
| 78 | 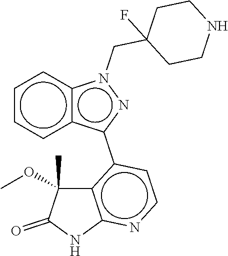 | Indazole 1 (using compound II′ obtained from OHMe isomer 2) | 410.1 | 1H NMR (DMSO-d6, 600 MHz): δ (ppm) 11.32 (s, 1H), 8.54-9.01 (m, 2H), 8.31 (d, J = 5.4 Hz, 1H), 7.83 (d, J = 8.2 Hz, 1H), 7.79 (d, J = 8.7 Hz, 1H), 7.51 (ddd, J = 8.3, 7.1, 0.9 Hz, 1H), 7.36 (d, J = 5.4 Hz, 1H), 7.27 (td, J = 7.5, 0.7 Hz, 1H), 4.82- 4.98 (m, 2H), 3.24-3.33 (m, 2H), 2.93-3.05 (m, 2H), 2.74 (s, 3H), 1.85-2.17 (m, 4H), 1.68 (s, 3H) |
| 79 | 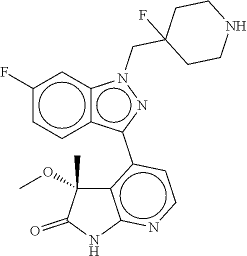 | Indazole 1 (using compound II′ obtained from OHMe isomer 2) | 428.1 | 1H NMR (DMSO-d6, 600 MHz): δ (ppm) 11.34 (s, 1H), 8.54-8.97 (m, 2H), 8.31 (d, J = 5.4 Hz, 1H), 7.85 (dd, J = 8.9, 5.0 Hz, 1H), 7.66 (d, J = 9.8 Hz, 1H), 7.34 (d, J = 5.4 Hz, 1H), 7.15 (td, J = 9.1, 2.3 Hz, 1H), 4.73- 4.99 (m, 2H), 3.23-3.33 (m, 2H), 2.92-3.08 (m, 2H), 2.75 (s, 3H), 1.84-2.15 (m, 4H), 1.65 (s, 3H) |
| 80 | 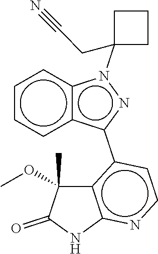 | Indazole 3 (using compound II′ obtained from OHMe isomer 2) | 388.1 | 1H NMR (DMSO-d6, 600 MHz): δ (ppm) 11.29 (s, 1H), 8.30 (d, J = 5.4 Hz, 1H), 7.89 (d, J = 8.2 Hz, 1H), 7.68 (d, J = 8.7 Hz, 1H), 7.47 (ddd, J = 8.3, 7.1, 0.9 Hz, 1H), 7.39 (d, J = 5.4 Hz, 1H), 7.25-7.32 (m, 1H), 3.39- 3.48 (m, 2H), 2.90-3.05 (m, 2H), 2.80 (s, 3H), 2.56-2.67 (m, 2H), 2.15-2.27 (m, 1H), 1.93-2.03 (m, 1H), 1.59 (s, 3H) |
| 81 | 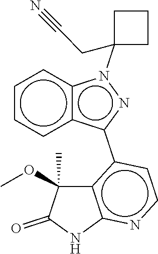 | Indazole 3 (using compound II′ obtained from OHMe isomer 1) | 388.1 | 1H NMR (DMSO-d6, 600 MHz): δ (ppm) 11.29 (s, 1H), 8.30 (d, J = 5.4 Hz, 1H), 7.89 (d, J = 8.2 Hz, 1H), 7.68 (d, J = 8.5 Hz, 1H), 7.47 (ddd, J = 8.4, 7.0, 1.0 Hz, 1H), 7.39 (d, J = 5.3 Hz, 1H), 7.24-7.33 (m, 1H), 3.39-3.49 (m, 2H), 2.86-3.07 (m, 2H), 2.80 (s, 3H), 2.55-2.67 (m, 2H), 2.14-2.26 (m, 1H), 1.93-2.04 (m, 1H), 1.59 (s, 3H) |
| 82 | 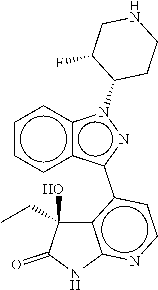 | Indazole 1 (using compound II′ obtained from EtOH isomer 1) | 396 | 1H NMR (DMSO-d6, 600 MHz): δ (ppm) 11.12 (br s, 1H), 8.28 (d, J = 5.4 Hz, 1H), 8.13 (d, J = 8.4 Hz, 1H), 7.99 (d, J = 8.7 Hz, 1H), 7.57 (ddd, J = 8.3, 7.1, 0.9 Hz, 1H), 7.55 (d, J = 5.6 Hz, 1H), 7.33-7.42 (m, 1H), 6.18 (s, 1H), 5.09-5.31 (m, 1H), 4.75-5.02 (m, 1H), 3.12-3.25 (m, 2H), 2.95- 3.08 (m, 1H), 2.77 (br t, J = 12.4 Hz, 1H), 2.43-2.49 (m, 1H), 2.01 (s, 1H), 1.77-1.97 (m, 3H), 0.42 (t, J = 7.6 Hz, 3H) |
| 83 | 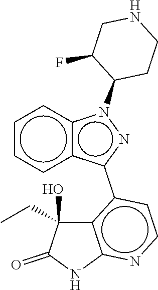 | Indazole 1 (using compound II′ obtained from EtOH isomer 1) | 396.1 | 1H NMR (DMSO-d6, 600 MHz): δ (ppm) 11.13 (br s, 1H), 8.29 (d, J = 5.6 Hz, 1H), 8.18 (d, J = 8.2 Hz, 1H), 8.00 (d, J = 8.7 Hz, 1H), 7.61 (d, J = 5.4 Hz, 1H), 7.58 (ddd, J = 8.3, 7.1, 0.9 Hz, 1H), 7.35-7.41 (m, 1H), 6.36 (s, 1H), 5.13-5.27 (m, 1H), 4.77-4.92 (m, 1H), 3.13-3.25 (m, 2H), 2.94- 3.07 (m, 1H), 2.79 (br t, J = 12.3 Hz, 1H), 2.50 (dt, J = 3.7, 1.8 Hz, 1H), 2.14 (s, 1H), 1.89 (br dd, J = 12.2, 2.5 Hz, 1H), 1.83 (q, J = 7.6 Hz, 2H), 0.43 (t, J = 7.5 Hz, 3H) |
| 84 | 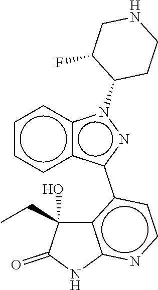 | Indazole 1 (using compound II′ obtained from EtOH isomer 2) | 396.1 | 1H NMR (DMSO-d6, 600 MHz): δ (ppm) 11.11 (br d, J = 0.6 Hz, 1H), 8.28 (d, J = 5.4 Hz, 1H), 8.13 (d, J = 8.2 Hz, 1H), 7.99 (d, J = 8.7 Hz, 1H), 7.57 (ddd, J = 8.4, 7.0, 0.9 Hz, 1H), 7.55 (d, J = 5.4 Hz, 1H), 7.35-7.40 (m, 1H), 6.18 (s, 1H), 5.11-5.23 (m, 1H), 4.81-4.97 (m, 1H), 3.12-3.25 (m, 2H), 2.91-3.07 (m, 1H), 2.77 (br t, J = 12.3 Hz, 1H), 2.40-2.49 (m, 1H), 1.97-2.10 (m, 1H), 1.77-1.97 (m, 3H), 0.42 (t, J = 7.6 Hz, 3H) |
| 85 | 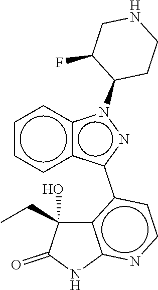 | Indazole 1 (using compound II′ obtained from EtOH isomer 2) | 396.1 | 1H NMR (DMSO-d6, 600 MHz): δ (ppm) 11.13 (br s, 1H), 8.29 (d, J = 5.6 Hz, 1H), 8.18 (d, J = 8.4 Hz, 1H), 8.00 (d, J = 8.7 Hz, 1H), 7.61 (d, J = 5.4 Hz, 1H), 7.58 (ddd, J = 8.3, 7.1, 0.9 Hz, 1H), 7.36-7.40 (m, 1H), 6.36 (s, 1H), 5.08-5.35 (m, 1H), 4.74-4.92 (m, 1H), 3.10-3.24 (m, 2H), 2.90- 3.07 (m, 1H), 2.79 (br t, J = 12.5 Hz, 1H), 2.50 (dt, J = 3.7, 1.8 Hz, 1H), 1.89 (br dd, J = 12.5, 2.3 Hz, 1H), 1.83 (q, J = 7.6 Hz, 2H), 0.43 (t, J = 7.5 Hz, 3H) |
| 86 | 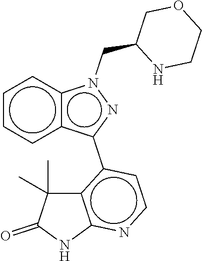 | Indazole 4 | 378.3 | 1H NMR (DMSO-d6, 600 MHz): δ (ppm) 11.10 (s, 1H), 8.22 (d, J = 5.4 Hz, 1H), 7.77-7.87 (m, 2H), 7.49 (ddd, J = 8.4, 6.9, 0.8 Hz, 1H), 7.30 (d, J = 5.4 Hz, 1H), 7.22-7.27 (m, 1H), 4.46 (d, J = 5.6 Hz, 2H), 3.53- 3.73 (m, 2H), 3.38 (td, J = 10.5, 2.4 Hz, 1H), 3.22-3.30 (m, 3H), 2.64- 2.93 (m, 2H), 1.36 (d, J = 16.1 Hz, 6H) |
| 87 | 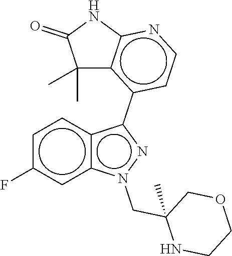 | Indazole 4 | 410.4 | 1H NMR (DMSO-d6, 500 MHz): δ (ppm) 10.98-11.25 (m, 1H), 8.22 (br d, J = 3.2 Hz, 1H), 7.77-7.87 (m, 1H), 7.72 (br d, J = 9.3 Hz, 1H), 7.27 (br s, 1H), 7.06-7.18 (m, 1H), 4.64 (br d, J = 14.9 Hz, 1H), 4.44-4.56 (m, 1H), 3.40-3.73 (m, 4H), 3.03-3.13 (m, 1H), 2.70-2.82 (m, 1H), 2.16-2.42 (m, 1H), 1.35 (br d, J = 1.5 Hz, 6H), 0.95 (br s, 3H) |
| 88 | 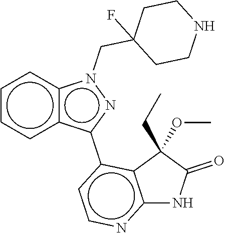 | Indazole 1 (synthesized from racemic and separated in chiral purification with method B, OMe/Et isomer 1) | 424.3 | 1H NMR (DMSO-d6, 500 MHz): δ (ppm) 11.01-11.60 (m, 1H), 8.30 (d, J = 5.4 Hz, 1H), 7.67-7.83 (m, 2H), 7.42-7.53 (m, 1H), 7.32 (d, J = 5.4 Hz, 1H), 7.24 (t, J = 7.5 Hz, 1H), 4.57-4.84 (m, 2H), 2.61-2.87 (m, 7H), 2.10-2.38 (m, 2H), 1.85-1.99 (m, 1H), 1.49-1.80 (m, 4H), 0.58 (t, J = 7.6 Hz, 3H) |
| 89 | 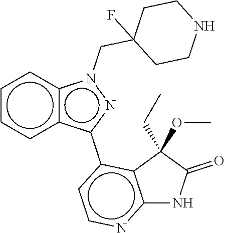 | Indazole 1 (synthesized from racemic and separated in chiral purification with method B, OMe/Et isomer 2) | 424.3 | 1H NMR (DMSO-d6, 500 MHz): δ (ppm) 10.85-11.72 (m, 1H), 8.30 (d, J = 5.4 Hz, 1H), 7.76 (t, J = 8.4 Hz, 2H), 7.47 (t, J = 7.8 Hz, 1H), 7.31 (d, J = 5.4 Hz, 1H), 7.24 (t, J = 7.5 Hz, 1H), 4.58-4.85 (m, 2H), 2.59-2.88 (m, 7H), 2.32 (dd, J = 12.6, 7.7 Hz, 1H), 1.82-2.19 (m, 2H), 1.47-1.81 (m, 4H), 0.58 (t, J = 7.6 Hz, 3H) |
| 90 | 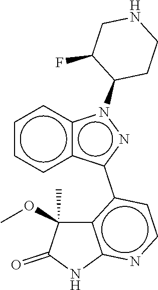 | Indazole 1(using compound II′ obtained from OHMe isomer 1) | 396.1 | 1H NMR (DMSO-d6, 600 MHz): δ (ppm) 11.23 (br s, 1H), 8.28 (d, J = 5.4 Hz, 1H), 7.90 (d, J = 8.7 Hz, 1H), 7.81 (d, J = 8.1 Hz, 1H), 7.47 (t, J = 7.7 Hz, 1H), 7.30 (d, J = 5.4 Hz, 1H), 7.26 (t, J = 7.6 Hz, 1H), 5.02-5.11 (m, 1H), 4,85 (d, J = 51.2 Hz, 1H), 3.13-3.22 (m, 2H), 2.98 (dd, J = 37.6, 13.2 Hz, 1H), 2.84 (s, 3H), 2.69-2.78 (m, 1H), 2.62-2.68 (m, 1H), 2.18 (br s, 1H), 1.84-1.92 (m, 1H), 1.54 (s, 3H) |
| 91 | 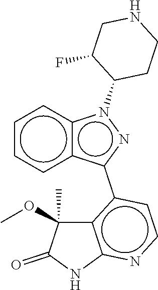 | Indazole 1(using compound II′ obtained from OHMe isomer 1) | 396.1 | 1H NMR (DMSO-d6, 600 MHz): δ (ppm) 11.25 (br s, 1H), 8.29 (d, J = 5.4 Hz, 1H), 7.91 (d, J = 8.5 Hz, 1H), 7.87 (d, J = 8.2 Hz, 1H), 7.48 (ddd, J = 8.3, 7.0, 1.0 Hz, 1H), 7.37 (d, J = 5.4 Hz, 1H), 7.27 (ddd, J = 8.0, 7.0, 0.7 Hz, 1H), 5.04-5.14 (m, 1H), 4.86 (d, J = 50.3 Hz, 1H), 3.15-3.22 (m, 2H), 2.98 (dd, J = 37.7, 14.1 Hz, 1H), 2.72-2.79 (m, 1H), 2.70 (s, 3H), 2.63-2.69 (m, 1H), 2.16 (br s, 1H), 1.89 (br dd, J = 12.3, 3.5 Hz, 1H), 1.73 (s, 3H) |
| 92 | 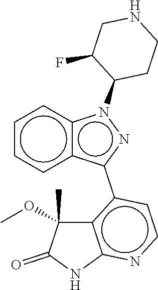 | Indazole 1(using compound II′ obtained from OHMe isomer 2) | 396 | 1H NMR (DMSO-d6, 600 MHz): δ (ppm) 11.25 (br s, 1H), 8.28 (d, J = 5.4 Hz, 1H), 7.90 (d, J = 8.7 Hz, 1H), 7.81 (d, J = 8.2 Hz, 1H), 7.48 (t, J = 7.7 Hz, 1H), 7.30 (d, J = 5.4 Hz, 1H), 7.26 (t, J = 7.6 Hz, 1H), 5.02-5.11 (m, 1H), 4.89 (d, J = 50.5 Hz, 1H), 3.15-3.27 (m, 2H), 2.98 (dd, J = 37.9, 13.9 Hz, 1H), 2.84 (s, 3H), 2.72-2.78 (m, 1H), 2.60-2.68 (m, 1H), 2.21 (br s, 1H), 1.85-1.91 (m, 1H), 1.54 (s, 3H) |
| 93 | 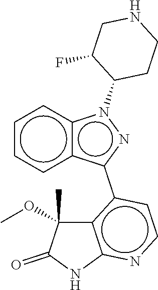 | Indazole 1(using compound II′ obtained from OHMe isomer 2) | 396.1 | 1H NMR (DMSO-d6, 600 MHz): δ (ppm) 11.26 (br s, 1H), 8.29 (d, J = 5.4 Hz, 1H), 7.91 (d, J = 8.5 Hz, 1H), 7.87 (d, J = 8.1 Hz, 1H), 7.48 (t, J = 7.5 Hz, 1H), 7.37 (d, J = 5.4 Hz, 1H), 7.27 (t, J = 7.6 Hz, 1H), 5.09 (br d, J = 19.8 Hz, 1H), 4.87 (d, J = 50.9 Hz, 1H), 3.15-3.21 (m, 2H), 2.98 (dd, J = 38.6, 15.3 Hz, 1H), 2.72-2.79 (m, 1H), 2.70 (s, 3H), 2.63-2.69 (m, 1H), 2.15 (br s, 1H), 1.89 (br dd, J = 12.5, 3.5 Hz, 1H), 1.73 (s, 3H) |
| 94 | 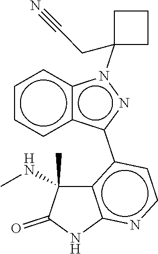 | Indazole 1 (using specific indazole 3 as compound III and synthesized from racemic and separated in chiral purification with method A, NHMe/Me isomer 2) | 387.3 | 1H NMR (600 MHz, DMSO-d6) δ ppm 11.10 (s, 1H), 0.00 (d, J = 5.43 Hz, 1H), 8.04-8.10 (m, 1H), 7.68 (d, J = 8.51 Hz, 1H), 7.51 (m, J = 5.40 Hz, 2H), 7.31-7.38 (m, 1H), 3.47- 3.58 (m, 2H), 2.96-3.07 (m, 1H), 2.86-2.95 (m, 2H), 2.59-2.69 (m, 2H), 1.94-2.29 (m, 2H), 1.88 (s, 3H), 1.39 (s, 3H) |
| 95 | 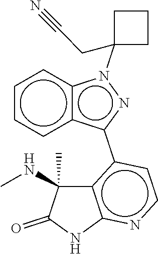 | Indazole 1 (using specific indazole 3 as compound III and synthesized from racemic and separated in chiral purification with method A, NHMe/Me isomer 1) | 387.3 | 1H NMR (600 MHz, DMSO-d6) δ ppm 11.10 (s, 1H), 8.25 (d, J = 5.58 Hz, 1H), 8.07 (d, J = 8.22 Hz, 1H), 7.65-7.72 (m, 1H), 7.44-7.58 (m, 2H), 7.29-7.37 (m, 1H), 3.47- 3.60 (m, 2H), 2.96-3.06 (m, 1H), 2.84-2.96 (m, 2H), 2.56-2.70 (m, 2H), 1.96-2.29 (m, 2H), 1.88 (s, 3H), 1.40 (s, 3H) |
| 96 | 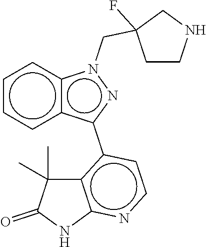 | Indazole 2 | 380.3 | 1H NMR (500 MHz, DMSO-d6) δ 11.14 (s, 1H), 9.83-9.17 (m, 2H), 8.23 (d, J = 5.4 Hz, 1H), 7.86 (d, J = 8.6 Hz, 1H), 7.83 (d, J = 8.3 Hz, 1H), 7.54 (t, J = 7.3 Hz, 1H), 7.33-7.24 (m, 2H), 5.22-5.03 (m, 2H), 3.72- 3.53 (m, 1H), 3.53-3.24 (m, 3H), 2.48-2.32 (m, 1H), 2.26-2.08 (m, 1H), 1.36 (d, J = 8.3 Hz, 6H) |
| 97 | 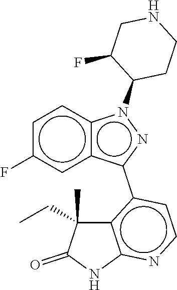 | Indazole 1 (using compound II′ obtained from MeEt isomer 2) | 412.3 | 1H NMR (DMSO-d6, 500 MHz): δ (ppm) 10.75-11.39 (m, 1H), 8.21 (d, J = 5.4 Hz, 1H), 8.01 (dd, J = 9.2, 4.3 Hz, 1H), 7.61 (dd, J = 9.0, 2.2 Hz, 1H), 7.43 (td, J = 9.0, 2.2 Hz, 1H), 7.31 (d, J = 5.4 Hz, 1H), 5.06-5.19 (m, 1H), 4.77-4.94 (m, 1H), 3.11- 3.26 (m, 2H), 2.92-3.07 (m, 1H), 2.75 (br t, J = 12.5 Hz, 1H), 2.61 (qd, J = 12.3, 3.9 Hz, 1H), 2.28 (br s, 1H), 1.99-2.09 (m, 1H), 1.89 (br d, J = 9.8 Hz, 1H), 1.61-1.71 (m, 1H), 1.45 (s, 3H), 0.34 (t, J = 7.3 Hz, 3H) |
| 98 | 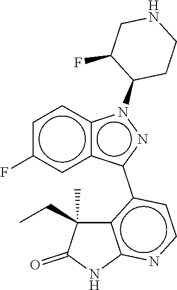 | Indazole 1 (using compound II′ obtained from MeEt isomer 1) | 412.3 | 1H NMR (600 MHz, DMSO-d6) δ 10.04-11.93 (m, 1H), 8.21 (d, J = 5.43 Hz, 1H), 8.00 (dd, J = 4.26, 9.24 Hz, 1H), 7.60 (dd, J = 2.27, 9.17 Hz, 1H), 7.42 (dt, J = 2.35, 9.02 Hz, 1H), 7.30 (d, J = 5.43 Hz, 1H), 5.07-5.16 (m, 1H), 4.77-4.93 (m, 1H), 3.11-3.22 (m, 2H), 2.93-3.04 (m, 1H), 2.75 (br t, J = 12.25 Hz, 1H), 2.60 (dq, J = 4.18, 12.40 Hz, 1H), 1.98-2.06 (m, 1H), 1.95-2.42 (m, 1H), 1.88 (br d, J = 9.24 Hz, 1H), 1.61-1.69 (m, 1H), 1.44 (s, 3H), 0.34 (t, J = 7.41 Hz, 3H) |
| 99 | 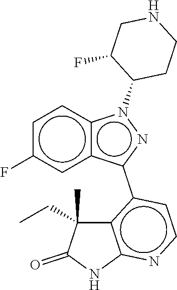 | Indazole 1 (using compound II′ obtained from MeEt isomer 2) | 412.3 | 1H NMR (600 MHz, DMSO-d6) δ 8.21 (d, J = 5.43 Hz, 1H), 8.00 (dd, J = 4.18, 9.17 Hz, 1H), 7.61 (dd, J = 2.35, 9.24 Hz, 1H), 7.42 (dt, J = 2.42, 9.06 Hz, 1H), 7.32 (d, J = 5.43 Hz, 1H), 5.07-5.17 (m, 1H), 4.77- 4.93 (m, 1H), 3.13-3.25 (m, 4H), 2.92-3.04 (m, 1H), 2.75 (br t, J = 12.47 Hz, 1H), 2.55-2.66 (m, 1H), 2.10-2.18 (m, 1H), 1.89 (br d, J = 9.68 Hz, 1H), 1.66-1.76 (m, 1H), 1.37 (s, 3H), 0.38 (t, J = 7.41 Hz, 3H) |
| 100 | 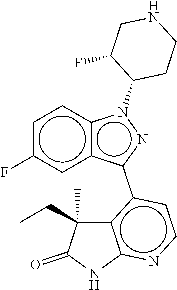 | Indazole 1 (using compound II′ obtained from MeEt isomer 1) | 412.3 | 1H NMR (600 MHz, DMSO-d6) δ 11.11 (br s, 1H), 8.21 (d, J = 5.43 Hz, 1H), 8.00 (dd, J = 4.25, 9.10 Hz, 1H), 7.61 (dd, J = 2.27, 9.17 Hz, 1H), 7.42 (dt, J = 2.42, 9.06 Hz, 1H), 7.32 (d, J = 5.43 Hz, 1H), 5.05-5.17 (m, 1H), 4.73-4.92 (m, 1H), 3.15-3.20 (m, 2H), 2.92-3.06 (m, 1H), 2.75 (br t, J = 12.32 Hz, 1H), 2.52-2.65 (m, 1H), 1.96-2.35 (m, 2H), 1.89 (br d, J = 9.54 Hz, 1H), 1.67-1.74 (m, 1H), 1.34-1.39 (m, 3H), 0.38 (t, J = 7.41 Hz, 3H) |
| 101 | 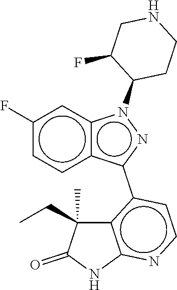 | Indazole 1 (using compound II′ obtained from MeEt isomer 1) | 412.3 | 1H NMR (DMSO-d6, 500 MHz): δ (ppm) 8.22 (d, J = 5.4 Hz, 1H), 7.82- 7.90 (m, 2H), 7.29 (d, J = 5.4 Hz, 1H), 7.15 (td, J = 9.0, 2.2 Hz, 1H), 4.98-5.10 (m, 1H), 4.77-4.93 (m, 1H), 3.12-3.22 (m, 3H), 2.91-3.06 (m, 2H), 2.69-2.79 (m, 1H), 2.55- 2.67 (m, 1H), 1.95-2.05 (m, 1H), 1.87 (br dd, J = 12.5, 2.9 Hz, 1H), 1.64 (dd, J = 13.4, 7.3 Hz, 1H), 1.44 (s, 3H), 0.33 (t, J = 7.5 Hz, 3H) |
| 102 | 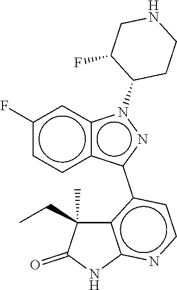 | Indazole 1 (using compound II′ obtained from MeEt isomer 1) | 412.3 | 1H NMR (DMSO-d6, 500 MHz): δ (ppm) 10.86-11.42 (m, 1H), 8.22 (d, J = 5.4 Hz, 1H), 7.80-7.93 (m, 2H), 7.31 (d, J = 5.6 Hz, 1H), 7.15 (td, J = 9.1, 2.1 Hz, 1H), 4.96-5.17 (m, 1H), 4.69-4.92 (m, 1H), 3.17 (br d, J = 11.5 Hz, 2H), 2.91-3.06 (m, 1H), 2.70-2.81 (m, 1H), 2.56-2.68 (m, 1H), 2.11 (dd, J = 13.7, 7.1 Hz, 1H), 2.00 (s, 1H), 1.88 (br dd, J = 12.6, 3.1 Hz, 1H), 1.69 (dd, J = 13.4, 7.3 Hz, 1H), 1.36 (s, 3H), 0.38 (t, J = 7.5 Hz, 3H) |
| 103 | 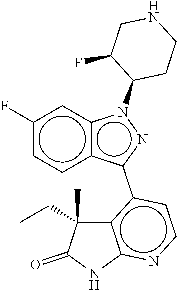 | Indazole 1 (using compound II′ obtained from MeEt isomer 2) | 412.3 | 1H NMR (DMSO-d6, 500 MHz): δ (ppm) 10.83-11.36 (m, 1H), 8.22 (d, J = 5.4 Hz, 1H), 7.84-7.89 (m, 2H), 7.31 (d, J = 5.4 Hz, 1H), 7.15 (td, J = 9.1, 2.1 Hz, 1H), 4,97-5.11 (m, 1H), 4.84 (d, J = 52.8 Hz, 1H), 3.13-3.22 (m, 2H), 2.91-3.06 (m, 1H), 2.69- 2.78 (m, 1H), 2.56-2.66 (m, 1H), 2.30 (br s, 1H), 2.08-2.16 (m, 1H), 1.84-1.91 (m, 1H), 1.65-1.74 (m, 1H), 1.36 (s, 3H), 0.38 (t, J = 7.5 Hz, 3H) |
| 104 | 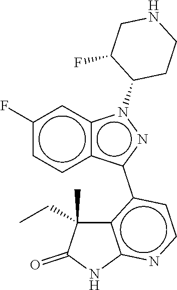 | Indazole 1 (using compound II′ obtained from MeEt isomer 2) | 412.3 | 1H NMR (DMSO-d6, 500 MHz): δ (ppm) 8.22 (d, J = 5.4 Hz, 1H), 7.83- 7.89 (m, 2H), 7.29 (d, J = 5.4 Hz, 1H), 7.15 (t, J = 8.8 Hz, 1H), 4.98- 5.11 (m, 1H), 4.78-4.93 (m, 1H), 3.12-3.23 (m, 3H), 2.93-3.04 (m, 2H), 2.69-2.77 (m, 1H), 2.55-2.67 (m, 1H), 2.00 (dd, J = 13.3, 7.5 Hz, 1H), 1.87 (br dd, J = 12.5, 2.9 Hz, 1H), 1.64 (dd, J = 13.4, 7.3 Hz, 1H), 1.44 (s, 3H), 0.33 (t, J = 7.3 Hz, 3H) |
| 105 | 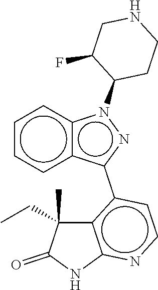 | Indazole 1 (using compound II′ obtained from MeEt isomer 2) | 394.3 | 1H NMR (600 MHz, DMSO-d6) δ 11.11 (br s, 1H), 8.22 (d, J = 5.4 Hz, 1H), 7.92 (d, J = 8.5 Hz, 1H), 7.86 (d, J = 8.2 Hz, 1H), 7.49 (t, J = 7.6 Hz, 1H), 7.33 (d, J = 5.4 Hz, 1H), 7.28 (t, J = 7.4 Hz, 1H), 5.15-5.05 (m, 1H), 4.85 (d, J = 51.2 Hz, 1H), 3.22-2.93 (m, 3H), 2.77 (br t, J = 12.3 Hz, 1H), 2.69-2.58 (m, 1H), 2.20-2.05 (m, 2H), 1.92-1.86 (m, 1H), 1.74- 1.66 (m, 1H), 1.38 (s, 3H), 0.38 (t, J = 7.4 Hz, 3H) |
| 106 | 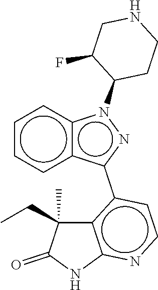 | Indazole 1 (using compound II′ obtained from MeEt isomer 1) | 394.1 | 1H NMR (DMSO-d6, 500 MHz) δ 11.11 (s, 1H), 8.22 (d, J = 5.4 Hz, 1H), 7.93 (d, J = 8.6 Hz, 1H), 7.85 (d, J = 8.3 Hz, 1H), 7.4-7.5 (m, 1H), 7.32 (d, J = 5.4 Hz, 1H), 7.28 (t, J = 7.6 Hz, 1H), 5.00-5.20 (m, 1H), 4.80-4.90 (m, 1H), 3.10-3.20 (m, 2H), 2.90- 3.10 (m, 1H), 2.77 (td, J = 1.0, 12.2 Hz, 1H), 2.65 (qd, J = 4.2, 12.5 Hz, 1H), 2.1-2.3 (m, 1H), 2.03 (spt, J = 7.6 Hz, 1H), 1.80-1.90 (m, 1H), 1.65 (spt, J = 7.3 Hz, 1H), 1.46 (s, 3H), 0.33 (t, J = 7.5 Hz, 3H) |
| 107 | 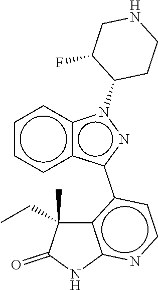 | Indazole 1 (using compound II′ obtained from MeEt isomer 2) | 394.1 | 1H NMR (DMSO-d6, 600 MHz): δ (ppm) 11.10 (br s, 1H), 8.22 (d, J = 5.4 Hz, 1H), 7.92 (d, J = 8.7 Hz, 1H), 7.85 (d, J = 8.2 Hz, 1H), 7.49 (ddd, J = 8.3, 7.1, 0.9 Hz, 1H), 7.31 (d, J = 5.3 Hz, 1H), 7.27 (td, J = 7.5, 0.7 Hz, 1H), 5.02-5.19 (m, 1H), 4.74-4.95 (m, 1H), 3.09-3.24 (m, 2H), 3.01 (dd, J = 38.7, 14.7 Hz, 1H), 2.76 (br t, J = 12.5 Hz, 1H), 2.59-2.69 (m, 1H), 2.13 (br dd, J = 4.2, 3.3 Hz, 1H), 1.99-2.07 (m, 1H), 1.89 (br dd, J = 12.6, 2.9 Hz, 1H), 1.59-1.70 (m, 1H), 1.45 (s, 3H), 0.33 (t, J = 7.3 Hz, 3H) |
| 108 | 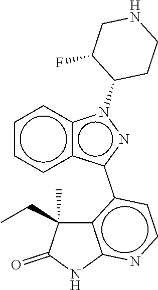 | Indazole 1 (using compound II′ obtained from MeEt isomer 1) | 394.1 | 1H NMR (600 MHz, DMSO-d6) δ 8.22 (d, J = 5.4 Hz, 1H), 7.92 (d, J = 8.5 Hz, 1H), 7.86 (d, J = 8.2 Hz, 1H), 7.49 (ddd, J = 8.3, 7.1, 0.9 Hz, 1H), 7.33 (d, J = 5.4 Hz, 1H), 7.28 (ddd, J = 8.0, 7.0, 0.7 Hz, 1H), 5.16-5.05 (m, 1H), 4.85 (d, J = 51.4 Hz, 1H), 3.29-3.27 (m, 1H), 3.22-3.15 (m, 2H), 3.07-2.91 (m, 2H), 2.77 (br t, J = 12.3 Hz, 1H), 2.69-2.60 (m, 1H), 2.18-2.10 (m, 1H), 1.89 (br dd, J = 12.3, 2.8 Hz, 1H), 1.75-1.66 (m, 1H), 1.38 (s, 3H), 0.38 (t, J = 7.3 Hz, 3H) |
| 109 | 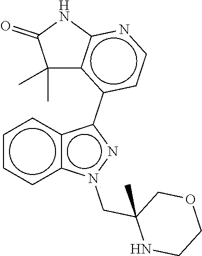 | Indazole 4 | 392.1 | 1H NMR (600 MHz, DMSO-d6) δ 11.10 (br s, 1H), 8.21 (d, J = 5.4 Hz, 1H), 7.83 (d, J = 8.5 Hz, 1H), 7.80 (d, J = 8.2 Hz, 1H), 7.49 (t, J = 7.7 Hz, 1H), 7.29 (d, J = 5.4 Hz, 1H), 7.24 (t, J = 7.6 Hz, 1H), 4.69 (d, J = 14.4 Hz, 1H), 4.53 (d, J = 14.4 Hz, 1H), 3.65 (br d, J = 11.0 Hz, 1H), 3.58 (d, J = 11.2 Hz, 1H), 3.52-3.46 (m, 1H), 3.29-3.26 (m, 1H), 3.13-3.07 (m, 1H), 2.74 (br d, J = 13.6 Hz, 1H), 2.38 (s, 1H), 1.36 (s, 6H), 0.96- 0.91 (m, 3H) |
| 110 | 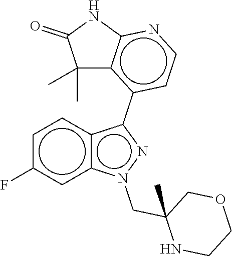 | Indazole 4 | 410.4 | 1H NMR (DMSO-d6, 500 MHz): δ (ppm) 11.13 (br s, 1H), 8.22 (d, J = 5.4 Hz, 1H), 7.81 (dd, J = 9.0, 5.1 Hz, 1H), 7.71 (dd, J = 9.9, 2.1 Hz, 1H), 7.26 (d, J = 5.4 Hz, 1H), 7.12 (td, J = 9.0, 2.2 Hz, 1H), 4.64 (d, J = 14.4 Hz, 1H), 4.49 (d, J = 14.4 Hz, 1H), 3.60- 3.67 (m, 1H), 3.55 (d, J = 11.2 Hz, 1H), 3.29-3.30 (m, 1H), 3.08 (ddd, J = 12.3, 8.6, 3.1 Hz, 1H), 2.71-2.79 (m, 1H), 2.21-2.39 (m, 1H), 1.35 (s, 6H), 1.06 (t, J = 7.0 Hz, 1H), 0.95 (s, 3H) |
| 111 | 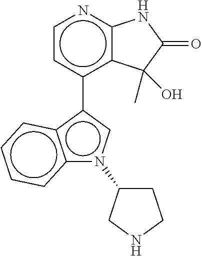 | Indole 1 | 349 | 1H NMR (DMSO-d6, 500 MHz): δ (ppm) 10.97 (s, 1H), 9.49 (br s, 2H), 8.45 (s, 1H), 8.14 (dd, J = 5.6, 2.0 Hz, 1H), 7.85 (dd, J = 7.9, 4.0 Hz, 1H), 7.69 (d, J = 8.1 Hz, 1H), 7.28- 7.34 (m, 2H), 7.19-7.25 (m, 1H), 5.42 (dt, J = 19.1, 7.2 Hz, 1H), 3.99 (br s, 2H), 3.18-3.60 (m, 4H), 2.53- 2.65 (m, 1H), 2.27-2.42 (m, 1H), 1.27 (s, 1,5H), 1.24 (s, 1,5H) |
| 112 | 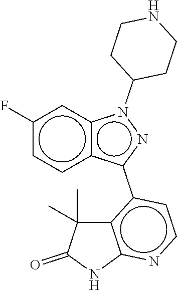 | Indazole 1 | 380 | 1H NMR (DMSO-d6, 500 MHz): δ (ppm) 11.16 (s, 1H), 8.66-9.16 (m, 2H), 8.23 (d, J = 5.4 Hz, 1H), 7.87 (dd, J = 9.0, 5.1 Hz, 1H), 7.78 (dd, J = 9.8, 2.2 Hz, 1H), 7.31 (d, J = 5.4 Hz, 1H), 7.16 (td, J = 9.0, 2.2 Hz, 1H), 5.07 (tt, J = 11.4, 3.9 Hz, 1H), 4.02 (br s, 2H), 3.51 (br d, J = 13.0 Hz, 2H), 3.06-3.21 (m, 2H), 2.28- 2.39 (m, 2H), 2.21 (br d, J = 12.2 Hz, 2H), 1.36 (s, 6H) |

### PKC-theta Activity, Prodrugs and Metabolites of Compounds

PKC-theta is selectively expressed in T lymphocytes and plays an important role in the T cell antigen receptor (TCR)-triggered activation of mature T cells, and the subsequent release of cytokines such as IL-2 and T cell proliferation (Isakov and Altman, Annu. Rev. Immunol., 2002, 20, 761-94). Thus, reduction of IL-2 levels is indicative of a desirable response that could provide a treatment of diseases and disorders as described herein, such as autoimmune and oncological disease.

Due to its involvement in T-cell activation, selective inhibition of PKC-theta may reduce harmful inflammation mediated by Th17 (mediating autoimmune diseases) or by Th2 (causing allergies) (Madouri et al, Journal of Allergy and Clinical Immunology. 139 (5): 2007, pp 1650-1666). without diminishing the ability of T cells to get rid of viral-infected cells. Inhibitors could be used in T-cell mediated adaptive immune responses. Inhibition of PKC-theta downregulates transcription factors (NF-κB, NF-AT) and results in lower production of IL-2. It was observed that animals without PKC-theta are resistant to some autoimmune diseases. (Zanin-Zhorov et al., Trends in Immunology. 2011, 32(8): 358-363). PKC-theta is therefore an interesting target for potential cancer and autoimmune therapies.

Studies in PKC-theta-deficient mice have demonstrated that while antiviral responses are independent of PKC-theta activity, T cell responses associated with autoimmune diseases are PKC-theta-dependent (Jimenez et al., J. Med. Chem. 2013, 56(5) pp 1799-1810). Thus, potent and selective inhibition of PKC-theta is expected to block autoimmune T cell responses without compromising antiviral immunity. However, the similarity of the PKC isoforms, particularly PKC-delta, and selectivity over other protein kinases represents a challenge to the development of a suitable PKC-inhibitor for clinical use.

In order to address such concerns, in aspects and embodiments, compounds (or ‘active agents’) of the disclosure may beneficially provide a potent and selective (having a selectivity of greater than 5-fold, preferably greater than 20-fold by a suitable measure, such as pIC50 in a suitable assay) PKC-theta inhibition over other PKC-isoforms, such as PKC-delta, and other kinases.

The active agents or compounds of the present invention may be provided as prodrugs of compounds of the disclosure.

The term ‘active agent’ is typically used to refer to a compound according to the disclosure which has inhibition activity against PKC-theta; especially under physiological conditions. However, it is often the case that the active agent may be difficult to administer or deliver to the physiological site of relevance, e.g. due to solubility, half-life or many other chemical or biological reasons. Therefore, it is known to use ‘prodrugs’ of the active agent in order to overcome physiochemical, biological or other barriers in drug efficiency and/or toxicity. Moreover, prodrug strategy may be used to increase the selectivity of drugs for their intended target. In accordance with the disclosure, therefore, prodrugs may be beneficial in targeting the active agent to the biological sites of interest while advantageously bypassing e.g. the stomach (or lungs), where problematic of inconvenient side-effects may be manifested due to localised inhibition of PKC-theta activity.

An active agent may be formed from a compound or prodrug of the disclosure by metabolism of the drug in vivo, and/or by chemical or enzymatic cleavage of the prodrug in vivo. Typically, a prodrug may be a pharmacologically inactive compound that requires chemical or enzymatic transformation to become an effective, active agent inside the body in which it is intended to have its therapeutic effect. On the other hand, since a prodrug may, in some embodiments, have very close structural similarity to the active agent, in some such embodiments, the prodrug may also have activity against the PKC-theta target. This may be particularly the case where the active agent is formed from a compound of prodrug of the disclosure by metabolism or a minor chemical transformation, such that the metabolite is closely related to the parent compound/prodrug. Accordingly, prodrugs of the disclosure may be active inhibitors of PKC-theta. Suitably, however, such prodrugs may be characterised by having lower inhibition activity against PKC-theta than the drug/active agent that is derived from the prodrug of the disclosure.

On the other hand, where the therapeutic effect is derived from the release of the active agent from a larger chemical entity, then the eventual active agent/compound/drug may have significant structural differences compared to the prodrug from which is was derived. In such cases, the prodrug can effectively ‘mask’ the form(s) of the active agent, and in such cases the prodrug may be completely (or essentially) completely inactive under physiological conditions.

### Dosage Forms, Medicaments and Pharmaceuticals

The compounds, molecules or agents of the disclosure may be used to treat (e.g. cure, alleviate or prevent) one or more diseases, infections or disorders. Thus, in accordance with the disclosure, the compounds and molecules may be manufactured into medicaments or may be incorporated or formulated into pharmaceutical compositions.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired. In general, a daily dosage of active agent of between about 0.001 and about 1,000 mg/kg of body weight can be expected. For some applications, the dosage may suitably be within the range of about 0.01 to about 100 mg/kg; between about 0.1 to about 25 mg/kg, or between about 0.5 and 10 mg/kg.

Depending on known factors, such as those noted above, the required dosage of the active agent may be administered in a single daily dose, or the total daily dosage may be administered in divided doses of e.g. two, three, or four times daily. Suitably, the therapeutic treatment regime according to the disclosure is devised for a single daily dose or for a divided daily dose of two doses.

Dosage forms of the pharmaceutical compositions of the disclosure suitable for administration may contain from about 1 mg to about 2,000 mg of the active ingredient per unit. Typically, the daily dosage of compounds may be at least about 10 mg and at most about 1,500 mg per human dose; such as between about 25 and 1,250 mg or suitably between about 50 and 1,000 mg. Typically, the daily dosage of compounds may be at most about 1000 mg. In such compositions the compound of the invention will ordinarily be present in an amount of about 0.5-95% by weight based on the total weight of the composition.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting PKC-theta.

Diseases or disorders which may benefit from PKC-theta inhibition include, for example, autoimmune disorders, inflammatory diseases, cancers and/or oncologic diseases, such as rheumatoid arthritis, multiple sclerosis, psoriasis, Sjogren's syndrome and systemic lupus erythematosus or vasculitic conditions, cancers of hematopoietic origin or solid tumors, including chronic myelogenous leukemia, myeloid leukemia, non-Hodgkin lymphoma and other B cell lymphomas.

For therapeutic applications, the effective amount or therapeutically effective amount of a compound/active agent of the disclosure may be at least about 50 nM or at least about 100 nM; typically at least about 200 nM or at least about 300 nM in the blood of the subject. The effective amount or therapeutically effective amount may be at most about 5 μM, at most about 3 μM, suitably at most about 2 μM and typically at most about 1 μM in the blood of the subject. For example, the therapeutically effective amount may be at most about 500 nM, such as between about 100 nM and 500 nM. In some embodiments the amount of therapeutic compound is measured in serum of the subject and the above concentrations may then apply to serum concentration of the compounds of the disclosure.

When administered to a subject, a compound of the disclosure is suitably administered as a component of a composition that comprises a pharmaceutically acceptable carrier or vehicle. One or more additional pharmaceutical acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound is to be administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the disclosure can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the disclosure are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach yet will release the material in the duodenum or elsewhere in the intestine. Suitably, the release will avoid the deleterious effects of the stomach environment, either by protection of the compound (or composition) or by release of the compound (or composition) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

While it can be beneficial to provide therapeutic compositions and/or compounds of the disclosure in a form suitable for oral administration, for example, to improve patient compliance and for ease of administration, in some embodiments compounds or compositions of the disclosure may cause undesirable side-effects, such as intestinal inflammation which may lead to premature termination of a therapeutic treatment regime. Thus, in some embodiments, the therapeutic treatment regime is adapted to accommodate ‘treatment holidays’, e.g. one or more days of non-administration. For example, treatment regimens and therapeutic methods of the disclosure may comprise a repetitive process comprising administration of the therapeutic composition or compound for a number of consecutive days, followed by a treatment holiday of one or more consecutive days. For example, a treatment regime of the disclosure may comprise a repetitive cycle of administration of the therapeutic composition or compound for between 1 and 49 consecutive days, between 2 and 42 days, between 3 and 35 days, between 4 and 28 days, between 5 and 21 days, between 6 and 14 days, or between 7 and 10 days; followed by a treatment holiday of between 1 and 14 consecutive days, between 1 and 12 days, between 1 and 10 days, or between 1 and 7 days (e.g. 1, 2, 3, 4, 5, 6 or 7 days).

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the disclosure is via pulmonary or nasal delivery.

Additives may be included to enhance cellular uptake of the therapeutic agent of the disclosure, such as the fatty acids oleic acid, linoleic acid and linolenic acid.

The therapeutic agents of the disclosure may also be formulated into compositions for topical application to the skin of a subject.

Where the invention provides more than one active compound/agent for use in combination, generally, the agents may be formulated separately or in a single dosage form, depending on the prescribed most suitable administration regime for each of the agents concerned. When the therapeutic agents are formulated separately, the pharmaceutical compositions of the invention may be used in a treatment regime involving simultaneous, separate or sequential administration with the other one or more therapeutic agent. The other therapeutic agent(s) may comprise a compound of the disclosure or a therapeutic agent known in the art).

The compounds and/or pharmaceutical compositions of the disclosure may be formulated and suitable for administration to the central nervous system (CNS) and/or for crossing the blood-brain barrier (BBB).

The invention will now be described by way of the following non-limiting examples.

### EXAMPLES

### Materials and Methods

Sample preparation: Powders were solubilized in DMSO-de, vortexed vigorously until the solution was clear and transferred to a NMR tube for data acquisition.

### NMR Spectroscopy:

Liquid-state NMR experiments were recorded on a 600 MHz (14.1 Tesla) Bruker Avance III NMR spectrometer (600 MHz for ¹H, 151 MHz for ¹³C) using a triple-resonance ¹H, ¹⁵N, ¹³C CP-TCI 5 mm cryoprobe (Bruker Biospin, Germany).

Liquid-state NMR experiments were recorded on a 500 MHz (11.75 Tesla) Bruker Avance I NMR spectrometer (500 MHz for ¹H, 125 MHz for ¹³C) using a Dual Resonance BBI 5 mm probe (Bruker Biospin, Germany).

Liquid-state NMR experiments were recorded on a 400 MHz (9.4 Tesla) Bruker Avance NEO NMR spectrometer (400 MHz for ¹H, 100 MHz for ¹³C) using a SEI 5 mm probe (Bruker Biospin, Germany).

All the experiments used for the resonance assignment procedure and the elucidation of the products structure (1D ¹H, 2D ¹H-¹H-COSY, 2D ¹H-¹H-ROESY, 2D ¹H-¹³C—HSQC, 2D ¹H-¹³C-HMBC) were recorded at 300 K. ¹H chemical shifts are reported in δ (ppm) as s (singlet), d (doublet), t (triplet), q (quartet), dd (double doublet), m (multiplet) or br s (broad singlet)

### LCMS Chromatography:

LCMS chromatography were recorded the following apparatus using:
Waters HPLC: Alliance 2695, UV: PDA 996, MS: ZQ (simple Quad) ZQ2
Waters UPLC: Acquity, UV: Acquity PDA, MS: Qda
Waters UPLC: Acquity, UV: Acquity TUV, MS: Qda
Waters UPLC: Acquity, UV: Acquity PDA, MS: QDa, ELSD

The apparatus was tested using a column Gemini NX-C18 Phenomenex (30×2 mm) 3 μm for the Waters HPLC or a CSH C18 Waters (50×2.1 mm), 1.7 μm for the UPLC Waters. All of them used a combination of the following eluents: H₂O+0.05% TFA (v/v) and ACN+0.035% TFA (v/v) and a positive electrospray ES+ as ionization mode. The UV detection was set up at 220 and 254 nm.

Temperatures are given in degrees Celsius (° C.). The reactants used in the examples below may be obtained from commercial sources or they may be prepared from commercially available starting materials as described herein or by methods known in the art. All of the compounds of the invention are synthesized according to the Examples described herein. The progress of the reactions described herein were followed as appropriate by e.g. LC, GC or TLC, and as the skilled person will readily realise, reaction times and temperatures may be adjusted accordingly.

### Chiral Purification:

### Method A:

Instrument: Waters Prep SFC80
Stationary Phase: Chiralpak IC 5 μm, 250×20 mm
Mobile phase: CO2/(EtOH+0.5% IPAm) 70/30
Flowrate: 50 ml/min
UV detection: 220 nm
Temperature: 40° C.
Pressure: 100 bars

### Method B:

Instrument: Waters Prep SFC80;
Stationary Phase: Chiralcel OJ-H 5 μm, 250×20 mm
Mobile phase: CO2/(EtOH+0.5% IPAm) 70/30
Flowrate: 50 mL/min
UV detection: λ=254 nm
Temperature: 40° C.—Pressure: 100 bars

### Abbreviations

In addition to the definitions above, the following abbreviations are used in the synthetic schemes above and the examples below. If an abbreviation used herein is not defined, it has its generally accepted meaning:
Ac Acetyl
ACN Acetonitrile
AcOH Acetic acid
BH₃ Borane
Boc tert-butyloxycarbonyl
B(OiPr)₃ Triisopropyl borate
CMBP Cyanomethylenetributylphosphorane
CS₂CO₃ Cesium carbonate
DAST Diethylaminosulfur trifluoride
DBU 1,8-Diazabicyclo(5.4.0)undec-7-ene
DCM Dichloromethane
DIPEA Diisopropylethylamine
DMAP 4-Dimethylaminopyridine
DMF Dimethylformamide
DMSO Dimethylsulfoxide
Et
Ethyl
Ethyl acetate
EtOAc
Et₃N Triethylamine
EtOH Ethanol
Et₂O Diethyl ether
h hour
H₂O water
HCl Hydrochloric acid
iPrOH Isopropanol
K₃PO₄ tripotassium phosphate
KOAC Potassium acetate
KOH Potassium hydroxide
min minutes
Me Methyl
MeCN Acetonitrile
MeO or OMe Methoxy
MeOH Methanol
MgSO₄ Magnesium sulfate
MS Mass spectrometry
Ms Mesyl
NaOH Sodium hydroxide
Na₂SO₄ Sodium sulfate
NaHCO₃Sodium bicarbonate
Na₂CO₃ Sodium carbonate
nBuLi n-Butyl Lithium
NBS N-Bromosuccinimide
NH₄Cl Ammonium chloride
NH₄HCO₃ Ammonium hydrogenocarbonate
Pd(PPh₃)₄ tetrakistriphenylphosphine palladium
Pd(dppf)Cl₂ bis(diphenylphosphino)ferrocene] dichloropalladium(II)
Pd/C Palladium on carbon
Ph Phenyl
rt Room temperature (18 to 22° C.)
TBAF tetrabutylammonium fluoride
TBDPSCl tert-butyl-chlorodiphenylsilane
TFA Trifluoroacetic acid
THF Tetrahydrofuran
Xantphos 4,5-Bis(diphenylphosphino)-9,9-dimethylxanthene
XPhos Dicyclohexyl[2′,4′,6′-tris(propan-2-yl)[1,1′-biphenyl]-2-yl]phosphane

### Example 1—Chemical Synthesis Routes

### Scaffolds

### Dimethyl Scaffold Synthesis

### Synthesis of 4-bromo-3,3-dimethyl-1H-pyrrolo[2,3-b]pyridin-2-one

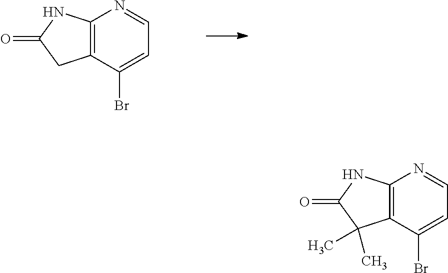

In a 250 ml three-necked round bottom flask, 1 M lithium bis(trimethylsilyl)amide solution (33 mL, 33.4 mmol, 3.8 eq.) was added dropwise via an additional funnel to a solution of 4-bromo-1,3-dihydro-2H-pyrrolo[2,3-b]pyridin-2-one (2.00 g, 8.92 mmol, 1 eq.) in anhydrous THF (44 mL, 0.2 N) at −78° C. The mixture was stirred at −78° C. for 10 min. Then iodomethane (1.4 mL, 22.3 mmol, 2.5 eq.) was added. The reaction was allowed to warm up to room temperature and stirred at room temperature for 1 h. Then NH₄Cl sat and ethyl acetate were added. The two phases were separated and the aqueous phase was extracted with ethyl acetate. Combined organic phases were dried over Na₂SO₄, filtered and evaporated to give crude product. The crude material was purified by flash chromatography on silica gel using a gradient of dichloromethane/ethyl acetate.

It was transferred via solid phase on dicalite. Relevant fractions were collected and concentrated under vacuum to afford 4-bromo-3,3-dimethyl-1H-pyrrolo[2,3-b]pyridin-2-one as a pale yellow powder (63% Yield). ¹H NMR (DMSO-d₆, 400 MHz): δ (ppm) 11.26 (s, 1H), 7.95 (d, J=5.7 Hz, 1H), 7.19 (d, J=5.7 Hz, 1H), 1.39 (s, 6H); m/z=241.2, 243.2 [M+H]+.

### Synthesis of 4-bromo-3,3-dimethyl-1-tetrahydropyran-2-yl-pyrrolo[2,3-b]pyridin-2-one

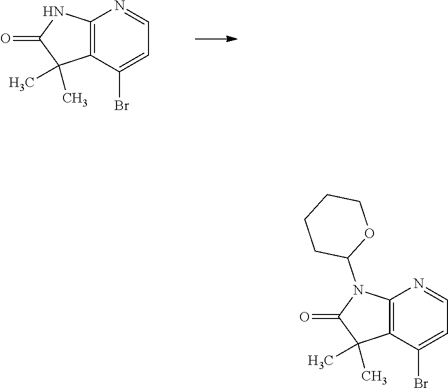 [SMILES: CC1(C)C(=O)Nc2nccc(Br)c21 ; CC1(C)C(=O)N(C2CCCCO2)c2nccc(Br)c21]

In a 20 mL microwave vial, 3,4-dihydro-2H-pyran (0.68 mL, 7.47 mmol, 3 eq.) was added to a stirred solution of 4-bromo-3,3-dimethyl-1H-pyrrolo[2,3-b]pyridin-2-one (600 mg, 2.49 mmol) and p-toluene sulfonic acid hydrate (95 mg, 0.498 mmol, 0.2 eq.) in anhydrous toluene (12 mL, 0.2 N). The reaction was stirred at 90″C for 5 h. The solvent was removed under vacuum to give crude material as an orange oil. The crude material was purified by flash chromatography on silica gel using a gradient of heptane/ethyl acetate. Relevant fractions were collected and concentrated under vacuum to afford 4-bromo-3,3-dimethyl-1-tetrahydropyran-2-yl-pyrrolo[2,3-b]pyridin-2-one (750 mg, 93% Yield). ¹H NMR (DMSO-d₆, 400 MHz): δ (ppm) 8.07 (d, J=5.6 Hz, 1H), 7.32 (d, J=5.6 Hz, 1H), 5.40 (dd, J=11.3, 2.1 Hz, 1H), 3.97 (d, J=10.8 Hz, 1H), 3.56 (qd, J=11.2, 10.8, 5.0 Hz, 1H), 2.85 (qd, J=13.7, 12.7, 3.8 Hz, 1H), 2.01-1.86 (m, 1H), 1.68-1.48 (m, 4H), 1.42 (s, 6H), m/z=325.2, 327.0 [M+H]+.

### Synthesis of 3,3-dimethyl-1-tetrahydropyran-2-yl-4-(4,4,5,5-tetramethyl-1,3,2-dioxaborolan-2-yl)pyrrolo[2,3-b]pyridin-2-one

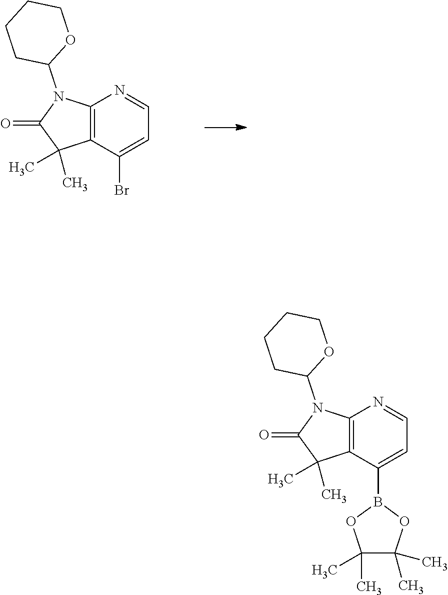 [SMILES: CC1(C)C(=O)N(C2CCCCO2)c2nccc(Br)c21 ; CC1(C)C(=O)N(C2CCCCO2)c2nccc(B3OC(C)(C)C(C)(C)O3)c21]

A sealed vial was charged under nitrogen with 4-bromo-3,3-dimethyl-1-tetrahydropyran-2-yl-pyrrolo[2,3-b]pyridin-2-one (0.75 g, 2.31 mmol), bis(pinacolato)diboron (0.88 g. 3.46 mmol, 1.5 eq.), potassium acetate (715 mg, 6.92 mmol, 3 eq.) and [1,1′-bis(diphenylphosphino)ferrocene] dichloropalladium(II), complex with dichloromethane (193 mg, 0.231 mmol, 0.1 eq.) in anhydrous dioxane (8 mL, 0.3 N). The vial was sealed and degassed with nitrogen. The reaction mixture was stirred at 100° C. overnight. The reaction mixture was filtered through a pad of dicalite and the filtrate was evaporated to dryness to give crude material as a dark oil. The crude product was purified by flash chromatography on silica gel using a gradient of heptane/ethyl acetate. Relevant fractions were collected and concentrated under vacuum to afford 3,3-dimethyl-1-tetrahydropyran-2-yl-4-(4,4,5,5-tetramethyl-1,3,2-dioxaborolan-2-yl)pyrrolo[2,3-b]pyridin-2-one (490 mg, 57% Yield) as a yellow oil. ¹H NMR (DMSO-d₆, 400 MHz): δ (ppm) 8.19 (d, J=5.1 Hz, 1H), 7.24 (d, J=5.1 Hz, 1H), 5.42 (dd, J=11.3, 2.0 Hz, 1H), 3.96 (d, J=11.1 Hz, 1H), 3.64-3.44 (m, 1H), 2.89 (d, J=11.4 Hz, 1H), 1.91 (s, 1H), 1.73-1.46 (m, 4H), 1.40 (s, 6H), 1.35 (s, 12H). m/z=373.4 [M+H]+.

### Ethyl/Methyl Scaffold Synthesis

### Synthesis of 3,4-dibromo-3-methyl-1H-pyrrolo[2,3-b]pyridin-2-one

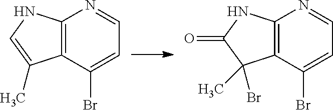

To a stirred solution of 4-bromo-3-methyl-1H-pyrrolo[2,3-b]pyridine (460 mg, 2.07 mmol) in tert-butanol (16 mL, 0.13 N) was added in small portions pyridinium bromide-perbromide (1.46 g, 4.56 mmol, 2.2 eq.) over 10 min. The reaction was stirred at room temperature overnight. t-Butanol was removed in vacuo. Water was added followed by ethyl acetate. The two phases were separated and the aqueous phase was extracted with EtOAc. Combined organic phases were washed with water, dried over Na₂SO₄, concentrated under high vacuum to give 3,4-dibromo-3-methyl-1H-pyrrolo[2,3-b]pyridin-2-one (660 mg, 96% Yield) as a white solid. ¹H NMR (DMSO-d₆, 400 MHz): δ (ppm) 11.77 (s, 1H), 8.04 (d, J=5.7 Hz, 1H), 7.32 (d, J=5.7 Hz, 1H), 2.07 (s, 3H); (product not stable in LCMS)

### Synthesis of 4-bromo-3-methyl-1,3-dihydropyrrolo[2,3-b]pyridin-2-one

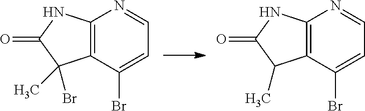

In a 50 mL round-bottomed flask, at room temperature, zinc powder (847 mg, 13.0 mmol, 2 eq.) was added in portions to a stirred suspension of 3,4-dibromo-3-methyl-1H-pyrrolo[2,3-b]pyridin-2-one (2.00 g, 6.01 mmol) in a mixture of methanol (30 mL) and acetic acid (15 mL). The reaction was stirred at room temperature for 10 min. The mixture was neutralized with an aqueous solution of NaHCO₃ until pH=6. The solution was filtered and the aqueous phase was extracted with EtOAc. Combined organic phases were washed with brine, dried over Na₂SO₄, filtered and evaporated to give 4-bromo-3-methyl-1,3-dihydropyrrolo[2,3-b]pyridin-2-one (1.08 g, 76% Yield) as a white solid. ¹H NMR (DMSO-d₆, 400 MHz): δ (ppm) 11.22 (s, 1H), 7.95 (dd, J=5.7, 0.8 Hz, 1H), 7.18 (d, J=5.7 Hz, 1H), 3.66-3.49 (m, 1H), 1.43 (d, J=7.6 Hz, 3H); m/z=227.1, 229.1 [M+H]+.

### Synthesis of 4-bromo-3-ethyl-3-methyl-1H-pyrrolo[2,3-b]pyridin-2-one

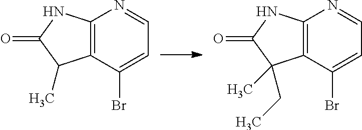

At −78° C., under an argon stream, 1 M lithium [bis(trimethylsilyl)amide] solution (2.2 mL, 2.16 mmol, 2 eq.) was added dropwise to a solution of 4-bromo-3-methyl-1,3-dihydropyrrolo[2,3-b]pyridin-2-one (350 mg, 1.08 mmol) in anhydrous tetrahydrofuran (2.7 mL, 0.4 N). The reaction was stirred at −78° C. for 10 min. Then iodoethane (0.087 mL, 1.08 mmol, 1 eq.) was added and the mixture was stirred at room temperature under argon stream for 1 h. Then an aqueous solution of HCl 1N was added slowly to reach pH6-7 followed by ethyl acetate. The two phases were separated and the aqueous phase was extracted with ethyl acetate. Combined organic phases were dried using a phase separator and evaporated to give crude material as an orange solid. The crude material was purified by flash chromatography on silica gel using a gradient of heptane/ethyl acetate. It was transferred via solid phase. Relevant fractions were collected and concentrated under vacuum to afford 4-bromo-3-ethyl-3-methyl-1H-pyrrolo[2,3-b]pyridin-2-one (155 mg, 56% Yield) as a beige powder. ¹H NMR (400 MHz, DMSO-d6) δ 11.30 (s, 1H), 7.96 (d, J=5.7 Hz, 1H), 7.21 (d, J=5.7 Hz, 1H), 2.21-2.05 (m, 1H), 1.77 (dq, J=14.7, 7.4 Hz, 1H), 1.38 (s, 3H), 0.50 (t, J=7.4 Hz, 3H); m/z=255.1, 257.1 [M+H]+.

The two enantiomers were obtained from chiral separation of the racemic mixture in SFC conditions.
Instrument: Novasep SFC Superprep
Stationary Phase: Chiralpak AD-H 20 μm, 300×50 mm
Mobile phase: CO2/MeOH 73/27
Flowrate: 1000 g/min UV detection: λ=295 nm
Temperature: 45° C.
Pressure: 130 bars
Sample: dissolution in MeOH
rt (MeEt isomer 1)=4.74 min and rt (MeEt isomer 2)=7.06 min

The S-isomer has been arbitrarily assigned as MeEt isomer 1 and the R-isomer has been arbitrarily assigned as MeEt isomer 2. The same nomenclature has been used to describe all related derivatives.

The following protocols were the same for racemic mixture and the pure enantiomers. The synthesis of boronic esters is described with the racemic mixture.

### Synthesis of 4-bromo-3-ethyl-3-methyl-1-tetrahydropyran-2-yl-pyrrolo[2,3-b]pyridin-2-one

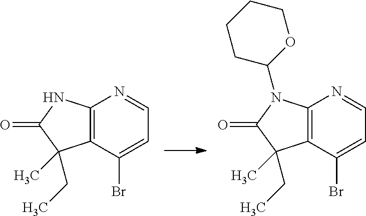 [SMILES: CCC1(C)C(=O)Nc2nccc(Br)c21 ; CCC1(C)C(=O)N(C2CCCCO2)c2nccc(Br)c21]

A 50 mL vial was charged with 4-bromo-3-ethyl-3-methyl-1H-pyrrolo[2,3-b]pyridin-2-one (2.14 g, 6.79 mmol), 3,4-dihydro-2H-pyran (1.9 mL, 20.4 mmol, 3 eq.), and p-toluene sulfonic acid hydrate (271 mg, 1.43 mmol, 0.2 eq) in anhydrous toluene (34 mL, 0.2 N). The reaction mixture was stirred at 80° C. overnight. The reaction mixture was allowed to reach room temperature. Then water was added and the reaction mixture was extracted with EtOAc. Combined organic layers were dried using a phase separator and concentrated under vacuum to give crude material as an orange solid. The crude material was purified by flash chromatography on silica gel using a gradient of Cyclohexane/EtOAc. It was transferred via solid phase on dicalite. Relevant fractions were collected and concentrated under vacuum to afford 4-bromo-3-ethyl-3-methyl-1-tetrahydropyran-2-yl-pyrrolo[2,3-b]pyridin-2-one (1.45 g, 62.951% Yield) as a yellow oil. ¹H NMR (400 MHz, DMSO-d6) δ 8.08 (d, J=5.6 Hz, 1H), 7.33 (d, J=5.7 Hz, 1H), 5.42 (dd, J=11.4, 1.8 Hz, 1H), 3.97 (d, J=10.9 Hz, 1H), 3.54 (tt, J=11.2, 2.9 Hz, 1H), 2.86 (pd, J=13.1, 3.9 Hz, 1H), 2.18 (ddh, J=15.0, 7.5, 3.5 Hz, 1H), 1.93 (d, J=10.8 Hz, 1H), 1.81 (dqd, J=14.7, 7.3, 1.7 Hz, 1H), 1.69-1.45 (m, 4H), 1.40 (d, J=0.8 Hz, 3H), 0.45 (t, J=7.4 Hz, 3H). m/z=338.9, 340.8 [M+H]+.

### Synthesis of 3-ethyl-3-methyl-1-tetrahydropyran-2-yl-4-(4,4,5,5-tetramethyl-1,3,2-dioxaborolan-2-yl)pyrrolo[2,3-b]pyridin-2-one

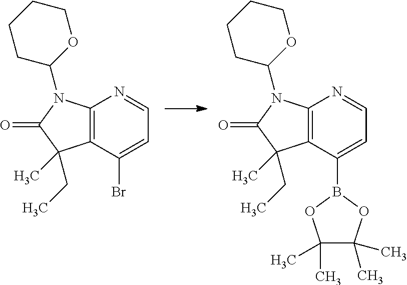 [SMILES: CCC1(C)C(=O)N(C2CCCCO2)c2nccc(Br)c21 ; CCC1(C)C(=O)N(C2CCCCO2)c2nccc(B3OC(C)(C)C(C)(C)O3)c21]

In a 20 mL microwave-vial were introduced bis(pinacolato)diboron (2.19 g, 8.61 mmol, 2 eq.), potassium acetate (1.33 g, 12.9 mmol, 3 eq.), 4-bromo-3-ethyl-3-methyl-1-tetrahydropyran-2-yl-pyrrolo[2,3-b]pyridin-2-one (1460 mg, 4.30 mmol) and [1,1′-bis(diphenylphosphino)ferrocene] dichloropalladium(II), complex with dichloromethane (352 mg, 0.430 mmol, 0.1 eq.) in anhydrous dioxane (43 mL, 0.1 N). The mixture was degassed with nitrogen and then stirred at 100° C. for 2 h. The reaction mixture was allowed to reach room temperature and filtered through a dicalite pad. The dicalite was washed with EtOAc. Combined organic layers were concentrated under vacuum to give crude material as a brown oil. The crude material was purified by flash chromatography on silica gel using a gradient of Cyclohexane/EtOAc. It was transferred via solid phase on dicalite. Relevant fractions were collected and concentrated under vacuum to afford 3-ethyl-3-methyl-1-tetrahydropyran-2-yl-4-(4,4,5,5-tetramethyl-1,3,2-dioxaborolan-2-yl)pyrrolo[2,3-b]pyridin-2-one (1.08 g, 52% Yield) as a pale yellow oil. ¹H NMR (DMSO-d₆, 400 MHz): δ (ppm) 8.19 (d, J=5.2 Hz, 1H), 7.25 (d, J=5.1 Hz, 1H), 5.43 (dd, J=11.4, 2.0 Hz, 1H), 3.96 (d, J=11.1 Hz, 1H), 3.64-3.49 (m, 1H), 3.01-2.79 (m, 1H), 2.33-2.16 (m, 1H), 1.93 (d, J=11.0 Hz, 1H), 1.87-1.73 (m, 2H), 1.71-1.43 (m, 6H), 1.34 (s, 12H), 0.38 (t. J=7.4 Hz, 3H); m/z=387.0 [M+H]+.

### Me/OH Scaffold Synthesis

### Synthesis of 4-bromo-3-hydroxy-3-methyl-1H-pyrrolo[2,3-b]pyridin-2-one

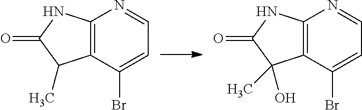

A round bottom flask was charged with sodium hydride (60%, 203 mg, 5.09 mmol, 1.1 eq.) in THF (10 mL) under N2. The mixture was cooled down to 0° C. and 4-bromo-3-methyl-1,3-dihydropyrrolo[2,3-b]pyridin-2-one (1.05 g, 4.62 mmol) in THF (13 mL) was added dropwise. Then the reaction was opened and left to the air overnight at room temperature. Then an aqueous solution of HCl 1N was added. The aqueous phase was extracted with ethyl acetate. Combined organic phases were dried over phase separator and evaporated to give crude material. The product was triturated in DCM to afford 4-bromo-3-hydroxy-3-methyl-1H-pyrrolo[2,3-b]pyridin-2-one (697 mg, 62% Yield) as a pale yellow solid. ¹H NMR (DMSO-d₆, 400 MHz): δ (ppm) 11.11 (s, 1H), 7.95 (d, J=5.7 Hz, 1H), 7.18 (d, J=5.7 Hz, 1H). 6.11 (s, 1H), 1.50 (s, 3H); m/z=243.1, 245.1 [M+H]+.

The two enantiomers were obtained from chiral separation of the racemic mixture in SFC conditions:
Instrument: Waters prep SFC Supersep
Stationary Phase: Chiralpak AD-H 20 μm, 250×50 mm
Mobile phase: CO2/MeOH 87/13
Flowrate: 1000 g/min UV detection: λ=290 nm
Temperature: 40° C.
Pressure: 150 bars
Sample: dissolution in MeOH
rt (OHMe isomer 1)=6.05 min and rt (OHMe isomer 2)=8.34 min

The S-isomer has been arbitrarily assigned as OHMe isomer 1 and the R-isomer has been arbitrarily assigned as OHMe isomer 2. The same nomenclature has been used to describe all related derivatives.

The following protocols were the same for racemic mixture or the pure enantiomers. The boronic esters synthesis will be described for enantiomer 1.

### Synthesis of (3R)-4-bromo-3-hydroxy-3-methyl-1-tetrahydropyran-2-yl-pyrrolo[2,3-b]pyridin-2-one

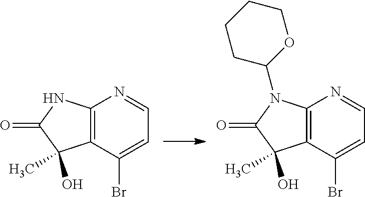 [SMILES: C[C@]1(O)C(=O)Nc2nccc(Br)c21 ; C[C@]1(O)C(=O)N(C2CCCCO2)c2nccc(Br)c21]

In a sealed vial, 3,4-dihydro-2H-pyran (3.0 mL, 32.9 mmol, 4 eq.) was added to a stirred solution of (3R)-4-bromo-3-hydroxy-3-methyl-1H-pyrrolo[2,3-b]pyridin-2-one (2.00 g, 8.23 mmol) and p-toluene sulfonic acid hydrate (313 mg, 1.65 mmol, 0.2 eq.) in anhydrous toluene (27 mL, 0.3 N). The reaction was stirred at 90° C. overnight. Then the mixture was cooled at 0° C. and 4 M hydrogen chloride (4.1 mL, 16.5 mmol, 2 eq.) was added. The mixture was stirred for 2 h at room temperature. The solution was concentrated under vacuum. Dichloromethane and an aqueous solution of NaHCO₃ aq were added. The aqueous phase was extracted by dichloromethane. The organic phase was dried on a phase separator and concentrated under vacuum. The crude material was purified by flash chromatography on silica gel using a gradient of heptane/EtOAc. Relevant fractions were collected and evaporated to afford (3R)-4-bromo-3-hydroxy-3-methyl-1-tetrahydropyran-2-yl-pyrrolo[2,3-b]pyridin-2-one (1.02 g, 36% Yield). ¹H NMR (DMSO-d₆, 400 MHz): δ (ppm) 8.07 (dd, J=5.6, 1.2 Hz, 1H), 7.31 (dd, J=5.7, 0.8 Hz, 1H), 6.28 (d, J=6.8 Hz, 1H), 5.37 (dd, J=11.3, 1.9 Hz, 1H), 4.02-3.90 (m, 1H), 3.54 (td, J=11.0, 10.6, 3.2 Hz, 1H), 2.90-2.73 (m, 1H), 1.93 (d, J=10.0 Hz, 1H), 1.69-1.44 (m, 7H); m/z=327.0, 328.9 [M+H]+.

### Synthesis of (3R)-3-hydroxy-3-methyl-1-tetrahydropyran-2-yl-4-(4,4,5,5-tetramethyl-1,3,2-dioxaborolan-2-yl)pyrrolo[2,3-b]pyridin-2-one

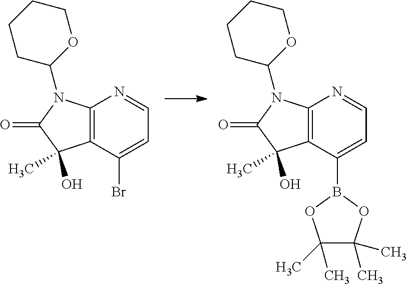 [SMILES: C[C@]1(O)C(=O)N(C2CCCCO2)c2nccc(Br)c21 ; CC1(C)OB(c2ccnc3c2[C@@](C)(O)C(=O)N3C2CCCCO2)OC1(C)C]

A vial was charged with bis(pinacolato)diboron (640 mg, 2.52 mmol, 1.5 eq.), potassium acetate (521 mg, 5.04 mmol, 3 eq.), (3R)-4-bromo-3-hydroxy-3-methyl-1-tetrahydropyran-2-yl-pyrrolo[2,3-b]pyridin-2-one (0.55 g, 1.68 mmol) and [1,1′-bis(diphenylphosphino)ferrocene] dichloropalladium(II), complex with dichloromethane (140 mg, 0.168 mmol, 0.1 eq.) in anhydrous dioxane (5.6 mL, 0.3N). The vial was sealed and degassed with nitrogen. The reaction mixture was stirred at 100° C. for 2 h. The reaction mixture was filtered through a pad of dicalite and the filtrate was evaporated to dryness to give crude material as a dark oil. The crude material was purified by flash chromatography on silica gel using a gradient of dichloromethane/ethyl acetate. It was transferred via solid phase on dicalite. Fractions were collected and concentrated under vacuum to afford (3R)-3-hydroxy-3-methyl-1-tetrahydropyran-2-yl-4-(4,4,5,5-tetramethyl-1,3,2-dioxaborolan-2-yl)pyrrolo[2,3-b]pyridin-2-one (211 mg, 28% Yield) was obtained as a yellow gum. ¹H NMR (DMSO-d₆, 400 MHz): δ (ppm) 8.18 (d, J=5.0 Hz, 1H), 7.14 (d, J=5.1 Hz, 1H), 5.92 (d, J=6.4 Hz, 1H), 5.38 (d, J=9.9 Hz, 1H), 3.96 (d, J=11.0 Hz, 1H), 3.59-3.49 (m, 1H), 2.86 (q, J=13.4, 12.5 Hz, 1H), 1.92 (s, 1H), 1.70-1.41 (m, 7H), 1.33 (d, J=7.0 Hz, 12H); m/z=293.2 [M+H]+.

### Me/OMe Scaffold Synthesis

### Synthesis of (3R)-4-bromo-3-methoxy-3-methyl-1-tetrahydropyran-2-yl-pyrrolo[2,3-b]pyridin-2-one

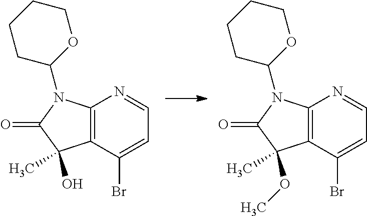 [SMILES: C[C@]1(O)C(=O)N(C2CCCCO2)c2nccc(Br)c21 ; CO[C@@]1(C)C(=O)N(C2CCCCO2)c2nccc(Br)c21]

In a 50 mL round-bottomed flask, at 0° C., under nitrogen, sodium hydride (60%, 378 mg, 9.44 mmol, 1.5 eq.) was added to a stirred solution of (3R)-4-bromo-3-hydroxy-3-methyl-1-tetrahydropyran-2-yl-pyrrolo[2,3-b]pyridin-2-one (2.06 g, 6.30 mmol) in anhydrous DMF (32 mL, 0.2 N). The reaction was stirred at room temperature for 30 mn. Then 2 M iodomethane in tert-butylmethyl ether (6.3 mL, 12.6 mmol, 2 eq.) was added dropwise at 0° C. The reaction was stirred at 0° C. for 15 min and allowed to reach room temperature. After 45 min at room temperature, the reaction was quenched with water and EtOAc was added. The two phases were separated and the aqueous phase was extracted with EtOAc. Combined organic phases were washed with water, dried using a phase separator and evaporated to give (3R)-4-bromo-3-methoxy-3-methyl-1-tetrahydropyran-2-yl-pyrrolo[2,3-b]pyridin-2-one as an orange gum (1.49 g, 63% Yield). ¹H NMR (DMSO-d₆, 400 MHz): δ (ppm) 8.16 (d, J=5.6 Hz, 1H), 7.40 (dd, J=5.6, 0.8 Hz, 1H), 5.42 (dt, J=11.4, 2.6 Hz, 1H). 4.00-3.93 (m, 1H), 3.61-3.49 (m, 1H), 2.91 (s, 3H), 2.87-2.75 (m, 1H), 1.94 (d, J=10.9 Hz, 1H), 1.70-1.41 (m, 7H); m/z=341.1, 343.1 [M+H]+.

### Synthesis of (3R)-3-methoxy-3-methyl-1-tetrahydropyran-2-yl-4-(4,4,5,5-tetramethyl-1,3,2-dioxaborolan-2-yl)pyrrolo[2,3-b]pyridin-2-one

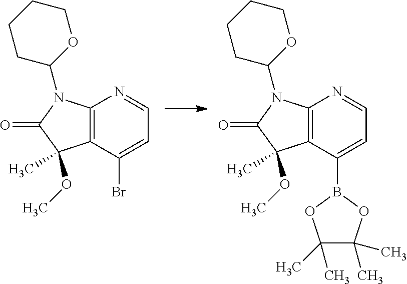 [SMILES: CO[C@@]1(C)C(=O)N(C2CCCCO2)c2nccc(Br)c21 ; CO[C@@]1(C)C(=O)N(C2CCCCO2)c2nccc(B3OC(C)(C)C(C)(C)O3)c21]

A reacti-vial, under a nitrogen atmosphere, was charged with tricyclohexylphosphane (459 uL, 0.290 mmol, 0.075 eq.), bis(pinacolato)diboron (1.96 g, 7.73 mmol, 4 eq.), (3R)-4-bromo-3-methoxy-3-methyl-1-tetrahydropyran-2-yl-pyrrolo[2,3-b]pyridin-2-one (1.45 g, 3.87 mmol) and anhydrous dioxane (19 mL, 0.2 N). Then potassium acetate (767 mg, 7.73 mmol, 4 eq.) and tris(dibenzylideneacetone)dipalladium(0) (186 mg, 0.193 mmol, 0.05 eq.) were added. The reaction was stirred at 100° C. for 2 h. The solvent was evaporated. Then water and dichloromethane were added. The two phases were separated and the aqueous phase was extracted with dichloromethane. Combined organic phases were dried using a phase separator and evaporated to give crude material as an orange gum. The crude material was purified by flash chromatography on silica gel using a gradient of heptane/ethyl acetate. It was transferred via solid phase. Relevant fractions were collected and concentrated under vacuum to afford (3R)-3-methoxy-3-methyl-1-tetrahydropyran-2-yl-4-(4,4,5,5-tetramethyl-1,3,2-dioxaborolan-2-yl)pyrrolo[2,3-b]pyridin-2-one (665 mg, 43% Yield) as an orange gum. ¹H NMR (DMSO-d₆, 400 MHz): δ (ppm) 8.26 (d, J=5.1 Hz, 1H), 7.22 (dd, J=5.1, 1.7 Hz, 1H), 5.42 (ddd, J=11.4, 5.4, 2.1 Hz, 1H), 4.01-3.94 (m, 1H), 3.62-3.48 (m, 1H), 2.89-2.76 (m, 4H), 1.94 (d, J=11.4 Hz, 1H), 1.73-1.46 (m, 7H), 1.33 (d, J=2.6 Hz, 12H); m/z=307.2 [M+H]+ (acid form).

### Et/OH Scaffold Synthesis

### Synthesis of 3-bromo-4-chloro-3-ethyl-1H-pyrrolo[2,3-b]pyridin-2-one

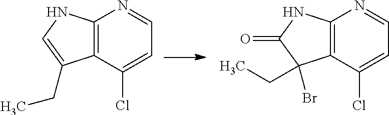

To a stirred solution of 4-chloro-3-ethyl-1H-pyrrolo[2,3-b]pyridine hydrochloride (3.00 g, 13.8 mmol) in tert-butanol (106 mL, 0.13 N) was added in small portions pyridinium bromide-perbromide (11.05 g, 34.5 mmol). The reaction was stirred at room temperature during 3 h. tert-butanol was removed under vacuum. The product was triturated in water and filtered to afford 3-bromo-4-chloro-3-ethyl-1H-pyrrolo[2,3-b]pyridin-2-one (2.95 g, 77% Yield) as a beige solid. 1H NMR (DMSO-d₆, 400 MHz): δ (ppm) 11.89 (s, 1H), 8.18 (d, J=5.7 Hz, 1H), 7.21 (d, J=5.7 Hz, 1H), 2.84-2.56 (m, 1H), 2.47-2.23 (m, 1H), 0.62 (t, J=7.4 Hz, 3H)

### Synthesis of 4-chloro-3-ethyl-1,3-dihydropyrrolo[2,3-b]pyridin-2-one

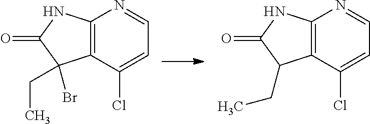

To a stirred suspension of 3-bromo-4-chloro-3-ethyl-1H-pyrrolo[2,3-b]pyridin-2-one (2.95 g, 10.7 mmol) in THF (33 mL, 0.3 N), at rt, was added zinc (1.05 g, 16.1 mmol) and then water (0.58 mL, 32.1 mmol) dropwise. The mixture was stirred at room temperature during 2 h. Then the solution was filtered under Dicalite to remove all residue of zinc. The filtrate was concentrated under vacuum to afford 4-chloro-3-ethyl-1,3-dihydropyrrolo[2,3-b]pyridin-2-one (2.1 g, 98% Yield) as a yellow solid; m/z=197.1, 199.1 [M+H]+.

### Synthesis of 4-chloro-3-ethyl-3-hydroxy-1H-pyrrolo[2,3-b]pyridin-2-one

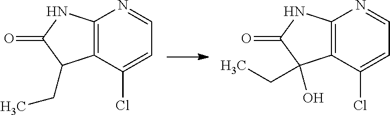

An aqueous solution of sodium hydroxide 10N (2.7 mL, 26.7 mmol) was added to a solution of 4-chloro-3-ethyl-1,3-dihydropyrrolo[2,3-b]pyridin-2-one (2.10 g, 10.7 mmol) in ethanol (49 mL, 0.2 N). The mixture was stirred at room temperature overnight. The mixture was concentrated under vacuum and a mixture of an aqueous solution of NH₄Cl and MeTHF was added. Phases were separated and the organic phase dried and concentrated under vacuum to afford 4-chloro-3-ethyl-3-hydroxy-1H-pyrrolo[2,3-b]pyridin-2-one (2.2 g, 94% Yield) as a yellow solid. ¹H NMR (400 MHz, DMSO-d₆) δ 8.07 (d, J=5.7 Hz, 1H), 7.06 (d, J=5.7 Hz, 1H), 6.19 (s, 1H), 2.13 (tt, J=14.3, 7.8 Hz, 1H), 2.03-1.87 (m, 1H), 0.55 (t, J=7.5 Hz, 3H); m/z=213.1, 215.1 [M+H]+.

The two enantiomers were obtained from chiral separation of the racemic mixture in SFC conditions:
Instrument: Waters prep SFC200
Stationary Phase: Chiralpak IC 5 μm, 250×30 mm
Mobile phase: CO2/MeOH 80/20
Flowrate: 100 mL/min UV detection: λ=210 nm
Temperature: 40° C.
Pressure: 100 bars
Sample: dissolution in MeOH
rt (OHEt isomer 1)=4.82 min and rt (OHEt isomer 2)=6.74 min

The S-isomer has been arbitrarily assigned as OHEt isomer 1 and the R-isomer has been arbitrarily assigned as OHEt isomer 2. The same nomenclature has been used to describe all related derivatives.

### Et/OMe Scaffold Synthesis

### Synthesis of 4-chloro-3-ethyl-3-hydroxy-1-tetrahydropyran-2-yl-pyrrolo[2,3-b]pyridin-2-one

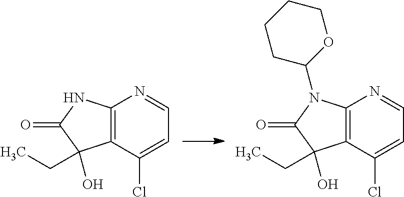 [SMILES: CCC1(O)C(=O)Nc2nccc(Cl)c21 ; CCC1(O)C(=O)N(C2CCCCO2)c2nccc(Cl)c21]

In a sealed vial, 3,4-dihydro-2H-pyran (0.79 mL, 8.67 mmol, 4 eq.) was added to a stirred solution of 4-chloro-3-ethyl-3-hydroxy-1H-pyrrolo[2,3-b]pyridin-2-one (614 mg, 2.89 mmol) and p-toluene sulfonic acid hydrate (110 mg, 0.578 mmol) in anhydrous toluene (12 mL, 0.2 N). The reaction was stirred at 90° C. overnight. Then the mixture was cooled at 0° C. and 4 M hydrogen chloride (1.4 mL, 5.78 mmol, 2 eq.) was added. The mixture was stirred 3 h at room temperature. The solution was concentrated under vacuum. Ethyl acetate and an aqueous solution of NaHCO₃ aq were added. The aqueous phase was extracted by ethyl acetate. The organic phase was dried on a phase separator and concentrated under vacuum. The crude material was purified by flash chromatography on silica gel using a gradient of heptane/EtOAc. Relevant fractions were collected and evaporated to afford 4-chloro-3-ethyl-3-hydroxy-1-tetrahydropyran-2-yl-pyrrolo[2,3-b]pyridin-2-one (446 mg, 52% Yield) as a yellow oil. ¹H NMR (DMSO-d₆, 400 MHz): δ (ppm) 8.19 (d, J=5.7 Hz, 1H), 7.18 (d, J=5.7 Hz, 1H), 6.34 (d, J=4.5 Hz, 1H), 5.39 (d, J=11.3 Hz, 1H), 3.97 (d, J=10.5 Hz, 1H), 3.55 (t, J=11.2 Hz, 1H), 2.92-2.73 (m, 1H), 2.17 (dtd, J=15.4, 7.7, 3.5 Hz, 1H), 1.99-1.88 (m, 2H), 1.64-1.44 (m, 4H), 0.50 (t, J=7.6 Hz, 3H); m/z=297.1, 299.1 [M+H]+.

### Synthesis of 4-chloro-3-ethyl-3-methoxy-1-tetrahydropyran-2-yl-pyrrolo[2,3-b]pyridin-2-one

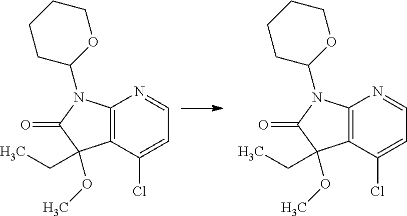 [SMILES: CCC1(OC)C(=O)N(C2CCCCO2)c2nccc(Cl)c21 ; CCC1(OC)C(=O)N(C2CCCCO2)c2nccc(Cl)c21]

To a solution of 4-chloro-3-ethyl-3-hydroxy-1-tetrahydropyran-2-yl-pyrrolo[2,3-b]pyridin-2-one (220 mg, 0.741 mmol) in anhydrous DMF (3.7 mL, 0.2 N) was added sodium hydride (60%, 44 mg, 1.11 mmol) at 0° C. under N₂. The resulting mixture was stirred 20 min at 0° C. Then iodomethane (0.092 mL, 1.48 mmol) was added dropwise at 0° C. The mixture was stirred 5 min at 0° C. and allowed to reach RT. The resulting mixture was stirred 30 min at RT under N₂. The mixture was quenched with water and extracted with EtOAc. The combined organic layers were washed with water and brine, dried over phase separator and concentrated under vacuum to afford 4-chloro-3-ethyl-3-methoxy-1-tetrahydropyran-2-yl-pyrrolo[2,3-b]pyridin-2-one (213 mg, 90% Yield) as a yellow oil. ¹H NMR (DMSO-d₆, 400 MHz): δ (ppm) 8.29 (d, J=5.7 Hz, 1H), 7.29 (dd, J=5.7, 1.2 Hz, 1H), 5.43 (d, J=11.3 Hz, 1H), 3.98 (d, J=11.0 Hz, 1H), 3.55 (1, J=11.1 Hz, 1H), 3.28 (d, J=4.8 Hz, 1H), 2.95 (d, J=1.2 Hz, 3H), 2.81 (d, J=11.4 Hz, 1H), 2.18 (ddd, J=13.2, 7.7, 2.4 Hz, 1H), 1.98 (dd, J=13.3, 7.5 Hz, 1H), 1.57 (d, J=45.7 Hz, 4H), 0.55 (1, J=7.5 Hz, 3H), m/z=311.2-313.2 [M+H]+.

### Scaffold Me/NMe

### Synthesis of 4-bromo-3-methyl-3-(methylamino)-1H-pyrrolo[2,3-b]pyridin-2-one

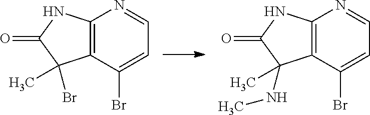

In a reactivial, a solution of methanamine in THF (6.0 mL, 8.04 mmol 1.34N) (cooled at −30° C.) was added at −30° C. to 3,4-dibromo-3-methyl-1H-pyrrolo[2,3-b]pyridin-2-one (500 mg, 1.63 mmol). The mixture was allowed to reach 0° C. and stirred 7 h at 0° C. The solution was concentrated to dryness to give a yellow gum. The crude material was purified by flash chromatography on silica gel using a gradient of heptane/EtOAc. It was transferred via liquid injection in DCM on a 24 g Redisep column. Relevant fractions were collected and concentrated under vacuum to afford 4-bromo-3-methyl-3-(methylamino)-1H-pyrrolo[2,3-b]pyridin-2-one as a white solid (179 mg, 43%); ¹H NMR (400 MHz, DMSO-d6) δ 11.23 (s, 1H), 7.96 (d, J=5.7 Hz, 1H), 7.20 (d, J=5.7 Hz, 1H), 1.90 (s, 3H), 1.41 (s, 3H); m/z=256.0, 258.0 [M+H]+

### Other Scaffolds

### Synthesis of 7-bromo-1,3-dihydroimidazo[4,5-b]pyridin-2-one

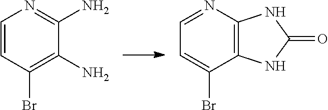 [SMILES: Nc1nccc(Br)c1N ; O=c1[nH]c2nccc(Br)c2[nH]1]

4-bromopyridine-2,3-diamine (5.00 g, 25.3 mmol) and 1,1′-carbonyldiimidazole (8.19 g, 50.5 mmol) were introduced to a sealed vial. THF (140 mL) was added and the mixture was stirred at 60° C. overnight. The flask was cooled with an ice-bath for 5 min. The precipitate was filtered through a glass-frit and washed once with cold THF followed by water. The solid was dried under vacuum. 7-bromo-1,3-dihydroimidazo[4,5-b]pyridin-2-one was afforded as a brown powder (5.14 g, 94%). ¹H NMR (DMSO-d₆, 400 MHz): δ (ppm) 11.60 (s, 1H), 11.39 (s, 1H), 7.74 (d, J=5.7 Hz, 1H), 7.17 (d, J=5.7 Hz, 1H); m/z=214.0, 216.0 [M+H]+.

### Synthesis of 7-bromo-3-tetrahydropyran-2-yl-1H-imidazo[4,5-b]pyridin-2-one

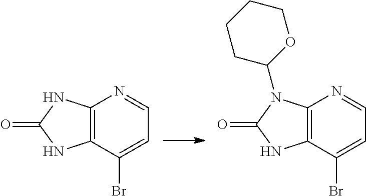 [SMILES: O=c1[nH]c2nccc(Br)c2[nH]1 ; O=c1[nH]c2c(Br)ccnc2n1C1CCCCO1]

To a solution of 7-bromo-1,3-dihydroimidazo[4,5-b]pyridin-2-one (500 mg, 2.34 mmol) in anhydrous THF (17.5 mL, 0.1 N) was added 3,4-dihydro-2H-pyran (0.64 mL, 7.01 mmol, 3 eq.) and p-toluene sulfonic acid hydrate (89 mg, 0.467 mmol, 0.2 eq.). The mixture was stirred at 75° C. overnight. 3,4-dihydro-2H-pyran (0.64 mL, 7.01 mmol, 3 eq.) was added and the reaction mixture was stirred at 75° C. for 3 h. The reaction was allowed to reach room temperature and quenched with water. EtOAc was added and the two layers were separated. Aqueous layer was extracted with EtOAc. Combined organic layer was dried over Na₂SO₄, filtered and concentrated under vacuum to give crude material as a brown oil. The crude mixture was purified by flash chromatography using a gradient of cyclohexane/EtOAc. It was transferred via solid deposit on dicalite. Relevant fractions were collected and concentrated under vacuum to afford 7-bromo-3-tetrahydropyran-2-yl-1H-imidazo[4,5-b]pyridin-2-one (452 mg, 65% Yield) as a yellow solid. 1H NMR (DMSO-d₆, 400 MHz): δ (ppm) 11.77 (s, 1H), 7.84 (d, J=5.6 Hz, 1H), 7.28 (d, J=5.7 Hz. 1H), 5.41 (dd, J=11.3, 2.2 Hz, 1H), 4.02-3.92 (m, 1H), 3.58 (td, J=11.3, 3.4 Hz, 1H), 2.94 (qd, J=12.6, 4.1 Hz, 1H), 1.99-1.90 (m, 1H), 1.76-1.45 (m, 4H); m/z=298.0; 300.0 [M+H]+.

### Synthesis of 3-tetrahydropyran-2-yl-7-(4,4,5,5-tetramethyl-1,3,2-dioxaborolan-2-yl)-1H-imidazo[4,5-b]pyridin-2-one

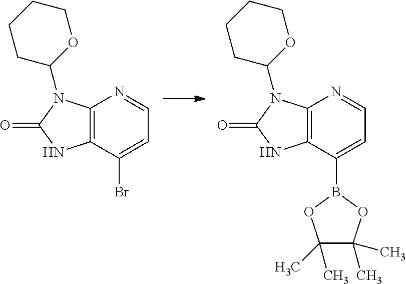 [SMILES: O=c1[nH]c2c(Br)ccnc2n1C1CCCCO1 ; CC1(C)OB(c2ccnc3c2[nH]c(=O)n3C2CCCCO2)OC1(C)C]

To a solution of 7-bromo-3-tetrahydropyran-2-yl-1H-imidazo[4,5-b]pyridin-2-one (300 mg, 1.01 mmol) in anhydrous dioxane (10 mL, 0.1 N) was added potassium acetate (420 mg, 4.02 mmol, 4 eq.) and bis(pinacolato)diboron (767 mg, 3.02 mmol, 3 eq.). The mixture was degassed with N₂ and [1,1′-bis(diphenylphosphino)ferrocene] dichloropalladium(II) (78 mg. 0.101 mmol, 0.1 eq.) was added. The resulting mixture was stirred 2 h at 95° C. under N₂. The mixture was filtered on dicalite and concentrated to give 3-tetrahydropyran-2-yl-7-(4,4,5,5-tetramethyl-1,3,2-dioxaborolan-2-yl)-1H-imidazo[4,5-b]pyridin-2-one (1.1 g, 57% Yield) as a dark oil. The crude material was engaged in next steps without more purification. m/z=264.1 [M+H]+. (boronic acid).

### Synthesis of 7-bromo-1-methyl-3-tetrahydropyran-2-yl-imidazo[4,5-b]pyridin-2-one

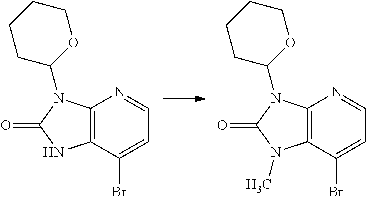 [SMILES: O=c1[nH]c2c(Br)ccnc2n1C1CCCCO1 ; Cn1c(=O)n(C2CCCCO2)c2nccc(Br)c21]

To a solution of 7-bromo-3-tetrahydropyran-2-yl-1H-imidazo[4,5-b]pyridin-2-one (502 mg, 1.63 mmol) in anhydrous DMF (8.3 mL, 0.1N) at 0° C. was added sodium hydride (78 mg, 1.95 mmol, 1.2 eq., 60%). The mixture was stirred for 15 min and iodomethane (125 μL, 2.01 mmol, 1.2 eq.) was added at the same temperature. The reaction mixture was stirred for 1 h. Water was added and the resulting precipitate was filtered and washed with water. The solid was dried at 40° C. under vacuum to afford 7-bromo-1-methyl-3-tetrahydropyran-2-yl-imidazo[4,5-b]pyridin-2-one (0.40 g, 77% Yield) as a pinkish solid. ¹H NMR (DMSO-d₆, 400 MHz): δ (ppm) 7.86 (d, J=5.6 Hz. 1H), 7.32 (d, J=5.6 Hz, 1H), 5.49 (dd, J=11.3, 2.2 Hz, 1H), 3.97 (dd, J=11.2, 2.0 Hz, 1H). 3.59 (s, 4H), 2.92 (qd, J=13.5, 13.0, 4.4 Hz, 1H), 2.03-1.89 (m, 1H), 1.79-1.41 (m, 4H); m/z=312.1, 314.1 [M+H]+.

### Synthesis of 7-bromo-3H-oxazolo[4,5-b]pyridin-2-one

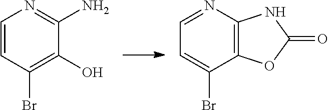 [SMILES: Nc1nccc(Br)c1O ; O=c1[nH]c2nccc(Br)c2o1]

2-amino-4-bromopyridin-3-ol (200 mg, 1.01 mmol) and 1,1′-carbonyldiimidazole (0.33 g, 2.01 mmol, 2 eq.) were introduced in a sealed vial. THF (6 mL, 0.2 N) was added and the mixture was stirred at 60° C. overnight. The solution was evaporated under vacuum and the crude triturated in DCM. The solid obtained was filtered and dried under vacuum to obtain 7-bromo-3H-oxazolo[4,5-b]pyridin-2-one as a brown powder (140 mg, 32% Yield). ¹H NMR (DMSO-d₆, 400 MHz): δ (ppm) 7.85 (d, J=5.8 Hz, 1H), 7.25 (d, J=5.8 Hz, 1H).

### Synthesis of 4-bromospiro[1H-pyrrolo[2,3-b]pyridine-3,1′-cyclopentane]-2-one

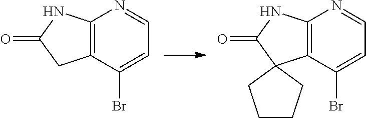 [SMILES: O=C1Cc2c(Br)ccnc2N1 ; O=C1Nc2nccc(Br)c2C12CCCC2]

A solution of 4-bromo-1,3-dihydro-2H-pyrrolo[2,3-b]pyridin-2-one (500 mg, 2.35 mmol) in anhydrous THF (7.8 mL, 0.3N) was cooled to −78° C. and 1 M lithium [bis(trimethylsilyl)amide] solution (8.2 mL, 8.21 mmol, 3.5 eq.) was added. After stirring for 30 minutes 1,4-diiodobutane (371 μL, 2.82 mmol, 1.2 eq.) was added dropwise. The reaction mixture was allowed to warm to room temperature and stirred overnight. The reaction was quenched with a saturated aqueous solution of NH₄Cl and extracted with EtOAc. The organic phase was dried using a phase separator and evaporated to give crude material as an oil. The crude material was purified by flash chromatography on silica gel using a gradient of heptane/EtOAc. It was transferred via solid phase on silica. Relevant fractions were collected and concentrated to give 4-bromospiro[1H-pyrrolo[2,3-b]pyridine-3,1′-cyclopentane]-2-one (258 mg, 41% yield). ¹H NMR (400 MHz, DMSO-d6) δ 11.12 (s, 1H), 7.91 (d, J=5.7 Hz, 1H), 7.19 (d, J=5.7 Hz, 1H), 2.15 (dd, J=8.1, 5.5 Hz, 2H), 2.08-1.82 (m, 6H); m/z=267.1, 269.1 [M+H]+.

### Synthesis of 4′-bromo-1′-tetrahydropyran-2-yl-spiro[cyclopentane-1,3′-pyrrolo[2,3-b]pyridine]-2′-one

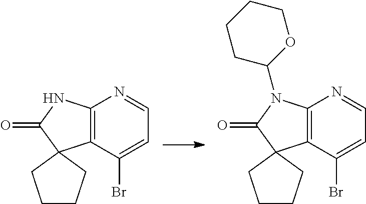 [SMILES: O=C1Nc2nccc(Br)c2C12CCCC2 ; O=C1N(C2CCCCO2)c2nccc(Br)c2C12CCCC2]

3,4-dihydro-2H-pyran (0.26 mL, 2.90 mmol, 3 eq.) was added to a stirred solution of 4-bromospiro[1H-pyrrolo[2,3-b]pyridine-3,1′-cyclopentane]-2-one (258 mg, 0.966 mmol) and p-toluene sulfonic acid hydrate (37 mg, 0.193 mmol, 0.2 eq.) in anhydrous toluene (4.8 mL, 0.2 N). The reaction was stirred at 90° C. overnight. The solvent was removed under vacuum. The crude material was purified by flash chromatography on silica gel using a gradient of heptane/ethyl acetate. Relevant fractions were collected and concentrated under vacuum to afford 4′-bromo-1′-tetrahydropyran-2-yl-spiro[cyclopentane-1,3′-pyrrolo[2,3-b]pyridine]-2′-one (238 mg, 70% Yield). ¹H NMR (400 MHz, DMSO-d₆) δ 8.04 (d, J=5.6 Hz, 1H), 7.32 (d, J=5.7 Hz, 1H), 5.37 (dd, J=11.3, 2.1 Hz, 1H), 3.96 (d, J=11.3 Hz, 1H), 3.53 (td, J=11.2, 4.0 Hz, 1H), 2.95-2.76 (m, 1H), 2.17 (dd, J=13.2, 5.9 Hz, 2H), 2.04-1.87 (m, 7H), 1.69-1.50 (m, 4H); m/z=351.2-353.2 [M+H]+.

### Synthesis of 1′-tetrahydropyran-2-yl-4′-(4,4,5,5-tetramethyl-1,3,2-dioxaborolan-2-yl)spiro[cyclopentane-1,3′-pyrrolo[2,3-b]pyridine]-2′-one

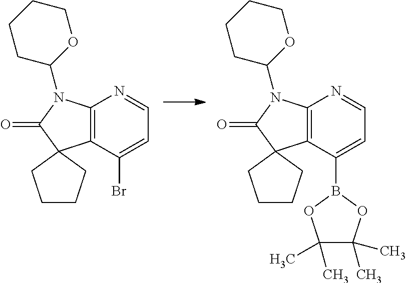 [SMILES: O=C1N(C2CCCCO2)c2nccc(Br)c2C12CCCC2 ; CC1(C)OB(c2ccnc3c2C2(CCCC2)C(=O)N3C2CCCCO2)OC1(C)C]

A vial was charged with bis(pinacolato)diboron (258 mg, 1.02 mmol, 1.5 eq.), potassium acetate (210 mg, 2.03 mmol, 3 eq.), 4′-bromo-1′-tetrahydropyran-2-yl-spiro[cyclopentane-1,3′-pyrrolo[2,3-b]pyridine]-2′-one (238 mg, 0.68 mmol) and [1,1′-bis(diphenylphosphino)ferrocene] dichloropalladium(II), complex with dichloromethane (57 mg, 0.068 mmol, 0.1 eq.) in anhydrous dioxane (2.2 mL, 0.3 N). The vial was sealed and degassed with nitrogen. The reaction mixture was stirred at 100° C. overnight. The reaction mixture was filtered through a pad of celite and the filtrate was evaporated to dryness to give crude material as a dark oil. The crude material was purified by flash chromatography on silica gel using a gradient of dichloromethane/ethyl acetate. It was transferred via solid phase on dicalite. Relevant fractions were collected and concentrated under vacuum to afford 1′-tetrahydropyran-2-yl-4′-(4,4,5,5-tetramethyl-1,3,2-dioxaborolan-2-yl)spiro[cyclopentane-1,3′-pyrrolo[2,3-b]pyridine]-2′-one (190 mg, 35% Yield). ¹H NMR (Chloroform-d, 400 MHz): δ (ppm) 8.16 (d, J=5.2 Hz, 1H), 7.28 (d, J=5.1 Hz, 1H), 5.52 (dd, J=11.3, 2.2 Hz, 1H), 4.21-4.10 (m, 1H), 3.69 (td, J=11.9, 2.2 Hz, 1H), 3.00 (qd, J=13.1, 12.6, 4.1 Hz, 1H), 2.29-1.95 (m, 9H), 1.85-1.60 (m, 4H), 1.35 (s, 12H); m/z=399.4 [M+H]+.

### Synthesis of 4-bromo-5-chloro-3,3-dimethyl-1H-pyrrolo[2,3-b]pyridin-2-one

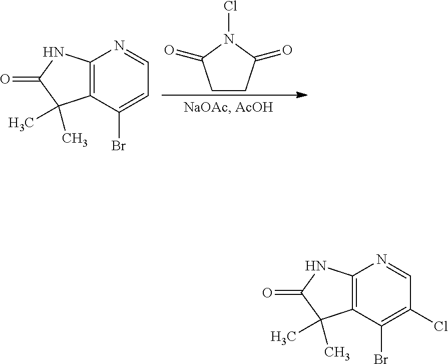

In a 50 mL round-bottomed flask, at room temperature, N-chlorosuccinimide (133 mg, 0.996 mmol, 1.6 eq.) was added to a stirred suspension of 4-bromo-3,3-dimethyl-1H-pyrrolo[2,3-b]pyridin-2-one (150 mg, 0.622 mmol) and sodium acetate (26 mg, 0.311 mmol, 0.5 eq.) in acetic acid (0.8 mL, 0.8 N). The mixture was heated at 60° C. for 2 h. N-chlorosuccinimide (133 mg, 0.996 mmol, 1.6 eq.) was added and the solution was stirred at 80° C. overnight. The reaction mixture was diluted with water and quenched with an aqueous solution of Na₂S₂O₃ 1M. The solid obtained was filtered through a glass-frit to give 4-bromo-5-chloro-3,3-dimethyl-1H-pyrrolo[2,3-b]pyridin-2-one (143 mg, 82% Yield) as a yellow powder. The product was engaged in next step without further purification. ¹H NMR (DMSO-d₆, 400 MHz): δ (ppm) 11.41 (s, 1H), 8.27 (s, 1H), 1.41 (s, 6H); m/z=275.0, 277.0 [M+H]+

### Synthesis of 4-chloro-5-fluoro-3,3-dimethyl-1H-pyrrolo[2,3-b]pyridin-2-one

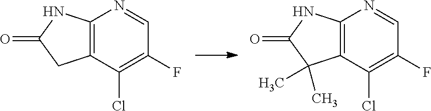

In a round-bottom flask, at 0° C., 1 M lithium [bis(trimethylsilyl)amide] solution (38 ml, 37.7 mmol, 3.7 eq.) was added dropwise to a stirred solution of 4-chloro-5-fluoro-1H,2H,3H-pyrrolo[2,3-b]pyridin-2-one (2.00 g. 10.2 mmol) in anhydrous 2-methyltetrahydrofuran (26 mL, 0.4 N). The mixture was stirred at 0° C. for 10 min. Then iodomethane (1.6 mL, 25.5 mmol, 2.5 eq.) was added dropwise at 0° C. and the mixture was stirred for 3 h at this temperature. An aqueous solution of NH₄Clₛₐₜ was added slowly. Water was added and the mixture was extracted with EtOAc. The combined organic layers were washed with water, brine, dried over phase separator and concentrated to afford a green solid. The crude product was taken in a mixture of diisopropylether/Et₂O (50/50) and filtered to afford 4-chloro-5-fluoro-3,3-dimethyl-1H-pyrrolo[2,3-b]pyridin-2-one (1.8 g, 78% Yield) as a green solid. ¹H NMR (DMSO-d₆, 400 MHz): δ (ppm) 11.32 (s, 1H), 8.24 (d, J=2.2 Hz, 1H), 1.41 (s, 6H). m/z=215.2, 217.2 [M+H]+

### Synthesis of 4-chloro-5-fluoro-3,3-dimethyl-1-tetrahydropyran-2-yl-pyrrolo[2,3-b]pyridin-2-one

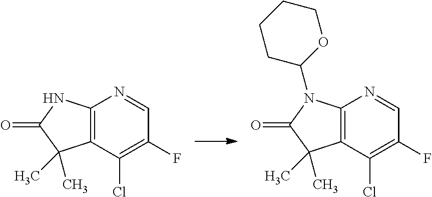 [SMILES: CC1(C)C(=O)Nc2ncc(F)c(Cl)c21 ; CC1(C)C(=O)N(C2CCCCO2)c2ncc(F)c(Cl)c21]

A 20 mL vial was successively charged with 4-chloro-5-fluoro-3,3-dimethyl-1H-pyrrolo[2,3-b]pyridin-2-one (830 mg, 3.87 mmol), anhydrous toluene (13 mL, 0.3 N), p-toluene sulfonic acid hydrate (147 mg, 0.773 mmol, 0.2 eq.) and 3,4-dihydro-2H-pyran (1.1 mL, 11.6 mmol, 3 eq). The reaction was stirred overnight at 90° C. Then 3,4-dihydro-2H-pyran (0.5 ml) was added and the reaction was stirred at 90° C. for another night. The solvent was evaporated to give crude material as a brown oil. The crude material was purified by flash chromatography on silica gel using a gradient of heptane/ethyl acetate. It was transferred via solid phase. Relevant fractions were collected and concentrated under vacuum to afford 4-chloro-5-fluoro-3,3-dimethyl-1-tetrahydropyran-2-yl-pyrrolo[2,3-b]pyridin-2-one (785 mg, 67% Yield) as an orange gum. 1H NMR (400 MHz, DMSO-d6) δ 8.37 (d, J=2.0 Hz, 1H), 5.38 (dd, J=11.3, 2.1 Hz, 1H), 3.97 (d, J=10.7 Hz, 1H), 3.55 (td, J=11.3, 4.0 Hz, 1H), 2.82 (qd, J=13.7, 12.9, 4.1 Hz, 1H), 1.97-1.88 (m, 1H), 1.69-1.48 (m, 4H), 1.44 (s, 6H), m/z=299.2, 301.2 [M+H]+

### Synthesis of 5-fluoro-3,3-dimethyl-1-tetrahydropyran-2-yl-4-(4,4,5,5-tetramethyl-1,3,2-dioxaborolan-2-yl)pyrrolo[2,3-b]pyridin-2-one

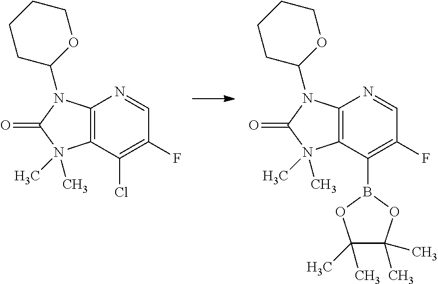

A reacti-vial, under nitrogen atmosphere, was charged with tricyclohexylphosphane (284 uL, 0.180 mmol, 0.075 eq.), bis(pinacolato)diboron (1.22 g. 4.79 mmol, 2 eq.), 4-chloro-5-fluoro-3,3-dimethyl-1-tetrahydropyran-2-yl-pyrrolo[2,3-b]pyridin-2-one (715 mg. 2.39 mmol) and anhydrous dioxane (12 mL, 0.2 N). Then potassium acetate (475 mg, 4.79 mmol, 2 eq) and tris(dibenzylideneacetone)dipalladium(0) (115 mg. 0.120 mmol, 0.05 eq.) were added. The reaction was stirred overnight at 100° C. The mixture was filtered on dicalite and concentrated to give crude material as a black oil. The crude material was purified by flash chromatography on silica gel using a gradient of heptane/ethyl acetate. 5-fluoro-3,3-dimethyl-1-tetrahydropyran-2-yl-4·(4,4,5,5-tetramethyl-1,3,2-dioxaborolan-2-yl)pyrrolo[2,3-b]pyridin-2·one (670 mg, 22% Yield) was obtained as a yellow solid (mixture of product and debrominated one). m/z=391.4 [M+H]+

### Synthesis of 5-fluoro-3-methyl-1,3-dihydropyrrolo[2,3-b]pyridin-2-one;hydrochloride

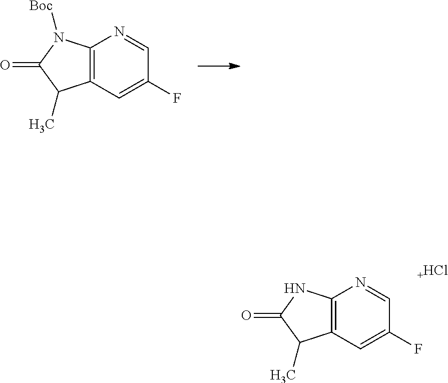

4 M hydrogen chloride in dioxane (1.0 mL, 4.00 mmol, 5 eq.) was added to a solution of tert-butyl 5-fluoro-3-methyl-2-oxo-3H-pyrrolo[2,3-b]pyridine-1-carboxylate (210 mg, 0.752 mmol) in anhydrous dioxane (2 mL, 0.3 N). The vial was sealed and the reaction was stirred at 60° C. for 1 h. The solution was concentrated to dryness to give 5-fluoro-3-methyl-1,3-dihydropyrrolo[2,3-b]pyridin-2-one;hydrochloride (139 mg, 84% yield) as a white solid. ¹H NMR (500 MHz, DMSO-d₆) δ 11.01 (br s, 1H), 8.03 (t, J=1.83 Hz, 1H), 7.69 (dd, J=2.20, 8.31 Hz, 1H), 3.54-3.61 (m, 1H), 1.35 (d, J=7.58 Hz, 3H); m/z=167.1 [M+H]+

### Synthesis of 3-ethyl-5-fluoro-3-methyl-1H-pyrrolo[2,3-b]pyridin-2-one

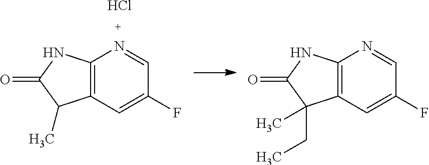

In a 2-5 mL vial, at 0° C., 1 M lithium [bis(trimethylsilyl)amide] solution (1.7 mL, 1.71 mmol, 3.8 eq.) was added dropwise via syringe to a stirred suspension of 5-fluoro-3-methyl-1,3-dihydropyrrolo[2,3-b]pyridin-2-one hydrochloride (98 mg, 0.445 mmol) in anhydrous 2-methyltetrahydrofuran (1.5 mL, 0.3 N). The reaction mixture was stirred at 0° C. for 10 min. iodoethane (0.065 mL, 0.813 mmol, 1.8 eq.) was added dropwise at 0° C. and the reaction was stirred at room temperature over the weekend. Water was added and the mixture was acidified with an aqueous solution of HCl to PH=5. EtOAc was added. The two phases were separated and the aqueous phase was extracted with EtOAc. Combined organic phases were washed with brine, dried using a phase separator and evaporated to give 3-ethyl-5-fluoro-3-methyl-1H-pyrrolo[2,3-b]pyridin-2-one (104 mg, 90% yield) as an orange solid. ¹H NMR (400 MHz, DMSO-d6) δ 11.05 (s, 1H), 8.05 (dd, J=2.7, 1.9 Hz, 1H), 7.75 (dd, J=8.3, 2.8 Hz, 1H), 1.86-1.69 (m, 2H), 1.28 (s, 3H), 0.57 (t, J=7.4 Hz, 3H). m/z=195.2 [M+H]+

### Synthesis of 3-ethyl-5-fluoro-3-methyl-1-tetrahydropyran-2-yl-pyrrolo[2,3-b]pyridin-2-one

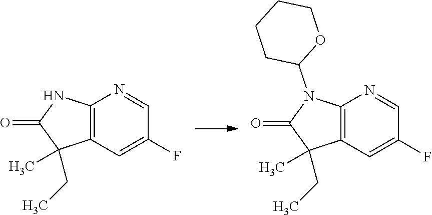 [SMILES: CCC1(C)C(=O)Nc2ncc(F)cc21 ; CCC1(C)C(=O)N(C2CCCCO2)c2ncc(F)cc21]

A 2-5 mL vial was charged with 3-ethyl-5-fluoro-3-methyl-1H-pyrrolo[2,3-b]pyridin-2-one (126 mg, 0.519 mmol), 3,4-dihydro-2H-pyran (0.14 mL. 1.56 mmol, 3 eq.) and p-toluene sulfonic acid hydrate (20 mg, 0.104 mmol, 0.2 N) in anhydrous toluene (1.7 mL, 0.3 N). The resulting mixture was stirred overnight at 95° C. and concentrated to dryness. The crude material was purified by flash chromatography on silica gel using a gradient of Heptane/EtOAc to afford 3-ethyl-5-fluoro-3-methyl-1-tetrahydropyran-2-yl-pyrrolo[2,3-b]pyridin-2-one (80 mg, 51% yield). ¹H NMR (DMSO-d₆, 600 MHz): δ (ppm) 8.17-8.18 (m, 1H), 7.85 (dd, J=8.2, 2.8 Hz, 1H), 5.36 (d, J=10.4 Hz, 1H), 3.95 (dt, J=11.4, 2.0 Hz, 1H), 3.53 (tt, J=11.4, 2.8 Hz, 1H), 2.79-2.94 (m, 1H), 1.89-1.95 (m, 1H), 1.74-1.86 (m, 2H), 1.53-1.65 (m, 2H), 1.45-1.55 (m, 2H), 1.29 (s, 3H), 0.51 (td, J=7.4, 3.4 Hz, 3H); m/z=279.2 [M+H]+.

### Synthesis of 5-ethyl-3-fluoro-5-methyl-7-tetrahydropyran-2-yl-4-(4,4,5,5-tetramethyl-1,3,2-dioxaborolan-2-yl)-7H-cyclopenta[b]pyridin-6-one

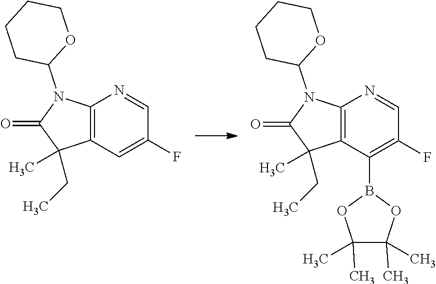 [SMILES: CCC1(C)C(=O)N(C2CCCCO2)c2ncc(F)cc21 ; CCC1(C)C(=O)N(C2CCCCO2)c2ncc(F)c(B3OC(C)(C)C(C)(C)O3)c21]

In a 2-5 mL vial, sealed, at −60° C. under N2, 1 M lithium diisopropylamide solution (0.60 mL, 0.600 mmol, 2.3 eq.) was added dropwise to a stirred solution of 3-ethyl-5-fluoro-3-methyl-1-tetrahydropyran-2-yl-pyrrolo[2,3-b]pyridin-2-one (78 mg, 0.256 mmol) in anhydrous THF (2 mL, 0.1 N). The reaction was stirred at −60° C. for 30 mn. triisopropyl borate (0.15 mL, 0.650 mmol, 2.5 eq.) was added dropwise at −60° C. The reaction was stirred at −60° C. for 30 mn and the mixture was allowed to warm to room temperature for 4 h. 2,3-dimethylbutane-2,3-diol (0.60 mL, 0.512 mmol, 2 eq) was added to the mixture then after 10 mn stirring, acetic acid (0.015 mL, 0.269 mmol, 1.05 eq) was added. The reaction was stirred at room temperature overnight. The mixture was filtered through dicalite. Solvent was partially evaporated under N2 stream and the solution extracted by an aqueous solution of NaOH 5%. The resulting aqueous layer was collected and acidified down to pH=6 at 0° C., by dropwise addition of 3N HCl, then extracted with EtOAc. Combined organic phases were washed with brine, dried using a phase separator and evaporated to give 5-ethyl-3-fluoro-5-methyl-7-tetrahydropyran-2-yl-4-(4,4,5,5-tetramethyl-1,3,2-dioxaborolan-2-yl)-7H-cyclopenta[b]pyridin-6-one (50 mg, 26% yield) as a brown gum. m/z=323.2 [M+H]+ (acid form) (impure)

### Scaffold Coupling—General Procedure (Indole)

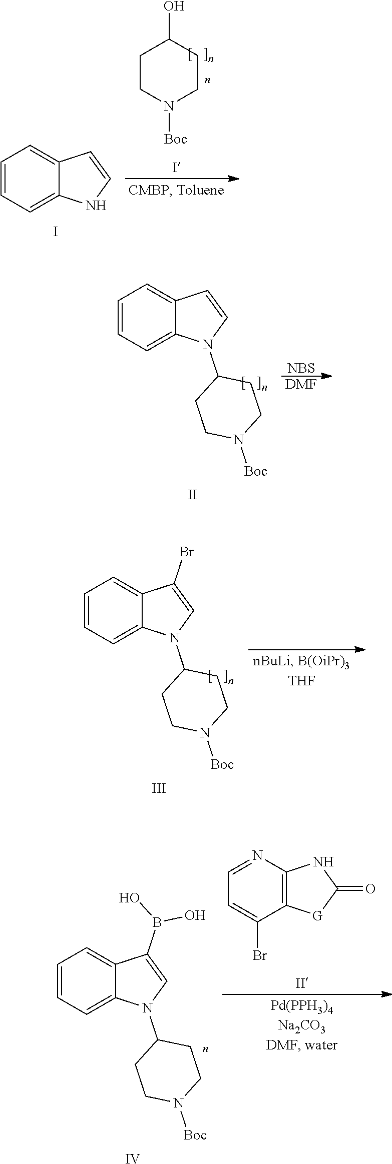

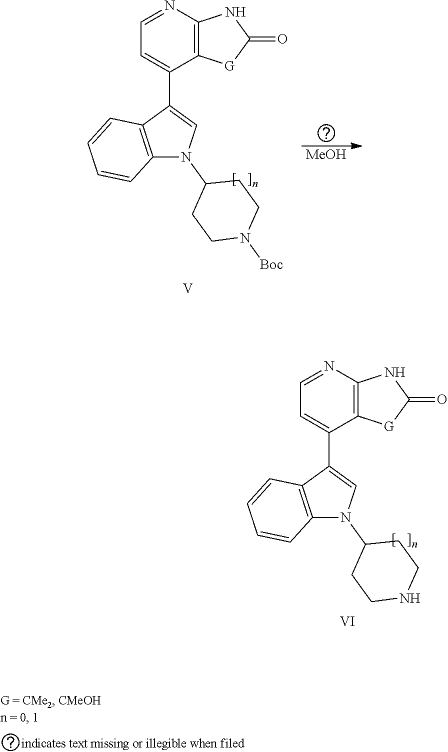

### 1. Mitsunobu Reaction

To a stirred mixture of indole I (1.66 mmol) in anhydrous toluene (8 mL, 0.2 N), were added cyanomethylenetributylphosphorane (3.31 mmol, 2 eq.) and alcohol I′ (2.48 mmol, 1.5 eq.). The reaction was stirred at 80° C. overnight. Cyanomethylenetributylphosphorane (3.31 mmol, 2 eq.) and alcohol I′ (2.48 mmol, 1.5 eq.) were added and the mixture stirred at 80° C. for a further 4 h. The reaction mixture was concentrated to dryness and the crude was purified by flash chromatography column with a gradient of EtOAc in cyclohexane. Relevant fractions were collected and concentrated under vacuum to afford expected products II.

### Example: Synthesis of tert-butyl 4-indol-1-ylpiperidine-1-carboxylate (n=1, G=CMe₂)

White oil, yield 82%, ¹H NMR (DMSO-d₆, 400 MHz): δ (ppm) 7.60-7.52 (m, 2H), 7.50 (d, J=3.2 Hz, 1H), 7.13 (t, J=7.8 Hz, 1H), 7.02 (t, J=7.4 Hz, 1H), 6.45 (d, J=3.2 Hz, 1H), 4.58 (ddt, J=11.7, 7.7, 3.8 Hz, 1H), 4.13 (d, J=12.2 Hz, 2H), 2.98 (s, 2H), 1.94 (d, J=10.4 Hz, 2H), 1.83 (qd, J=12.3, 4.3 Hz, 2H), 1.44 (s, 9H); m/z=245.3 [M+H-tBu]+

### 2. Bromination

N-bromosuccinimide (1.45 mmol, 1.05 eq.) was added to a solution of substituted indole II (1.38 mmol) in anhydrous DMF (13.8 mL, 0.1 N). The resulting mixture was stirred 6 h at room temperature under N₂. N-bromosuccinimide (1 eq.) was added and the reaction mixture was stirred at room temperature overnight under N₂. Water was added and the mixture was extracted with EtOAc. The combined organic layers were washed with water and brine, dried over phase separator and concentrated under vacuum. The crude product was purified on silica gel column with a gradient of heptane/EtOAc. Relevant fractions were collected and concentrated under vacuum to give brominated products III.

### Example: Synthesis of tert-butyl 4-(3-bromoindol-1-yl)piperidine-1-carboxylate (n=1, G=CMe₂)

White oil, yield 83%, ¹H NMR (DMSO-d₆, 400 MHz): δ (ppm) 7.77 (s, 1H), 7.65 (d, J=8.3 Hz, 1H), 7.42 (d, J=7.9 Hz, 1H), 7.28-7.20 (m, 1H), 7.16 (t, J=7.4 Hz, 1H), 4.75-4.51 (m, 1H), 4.26-3.91 (m, 2H), 2.96 (s, 2H), 2.07-1.73 (m, 4H), 1.44 (s, 9H); m/z=323.1, 325.1 [M+H-tBu]+

### 3. Boronic Acid Formation

To a solution of bromoindoles III (0.854 mmol) in anhydrous THF (4.3 mL, 0.2 N) was added dropwise 1.2 M butyllithium solution (1.1 mL, 1.28 mmol, 1.5 eq.) at −78° C. The resulting mixture was stirred for 20 min at −78° C. under N₂. Then triisopropyl borate (0.59 mL, 2.56 mmol, 3 eq.) was added and the solution stirred 4 h30 while temperature was allowed to rise to room temperature. A mixture of Water/MeOH (1:1.3 ml) was added to quench the reaction. Water was added and the mixture was extracted with diethyl ether. The organic phase was washed with brine, dried over phase separator and concentrated to give expected boronic acids IV.

### Example: Synthesis of [1-(1-tert-butoxycarbonyl-4-piperidyl)indol-3-yl]boronic acid (n=1, G=CMe₂) Green Solid, Yield 51%, m/z=245.3 [M+H-Boc]+

### 4. Suzuki Coupling

A reacti-vial was charged with boronic acid IV (0.218 mmol, 1.05 eq.), bromine scaffold II′ (0.207 mmol), disodium carbonate (0.622 mmol, 3 eq.) in a mixture of DMF (1.5 mL) and water (0.5 mL). The mixture was degassed and tetrakistriphenylphosphine palladium (0.0207 mmol, 0.1 eq.) was added. The resulting mixture was stirred overnight at 90° C. under N₂. The mixture was filtered on dicalite and evaporated under vacuum. Crude products were purified on silica gel column with a gradient of heptane/EtOAc. Relevant fractions were collected and concentrated under vacuum to afford Suzuki coupling products V.

### Example: Synthesis of tert-butyl 4-[3-(3,3-dimethyl-2-oxo-1H-pyrrolo[2,3-b]pyridin-4-yl)indol-1-yl]piperidine-1-carboxylate (n=1, G=CMe₂)

Beige solid; Yield 20%; m/z=461.4 [M+H]+

### 5. Deprotection

4 M hydrogen chloride solution in dioxane (0.16 mmol, 4 eq.) was added to a solution of Suzuki coupling compounds V (0.041 mmol) in anhydrous methanol (0.2 mL, 0.2 eq.). The reaction was stirred at room temperature overnight. Diisopropyl ether was added to the mixture and the precipitate was filtered to give a gum. The precipitate was diluted in MeOH and the solution concentrated under vacuum to give final compounds as hydrochloric salts.

### Example 7: Synthesis of 3,3-dimethyl-4-[1-(4-piperidyl)indol-3-yl]-1H-pyrrolo[2,3-b]pyridin-2-one Dihydrochloride (n=1, G=CMe₂)

Orange solid; yield 95%, ¹H NMR (DMSO-d₆, 500 MHz): δ (ppm) 11.09 (s, 1H), 8.55-9.02 (m, 2H), 8.11 (d, J=5.4 Hz, 1H), 7.71 (d, J=8.3 Hz, 1H), 7.51 (s, 1H), 7.35 (d, J=7.8 Hz, 1H), 7.26 (td, J=7.6, 1.0 Hz, 1H), 7.10 (td, J=7.3, 0.5 Hz, 1H), 6.92 (d, J=5.4 Hz, 1H), 4.85 (tt, J=11.5, 4.7 Hz, 1H), 3.48 (br d, J=13.4 Hz, 3H), 3.10-3.23 (m, 2H), 2.17-2.29 (m, 4H), 1.13 (s, 6H); m/z=361.1

### Scaffold Coupling—Specific Examples

The indole I was either obtained from commercial sources or synthesised by standard techniques according to the procedures that follow.

### Scaffold Coupling—Specific Procedure (Specific Indole 1)

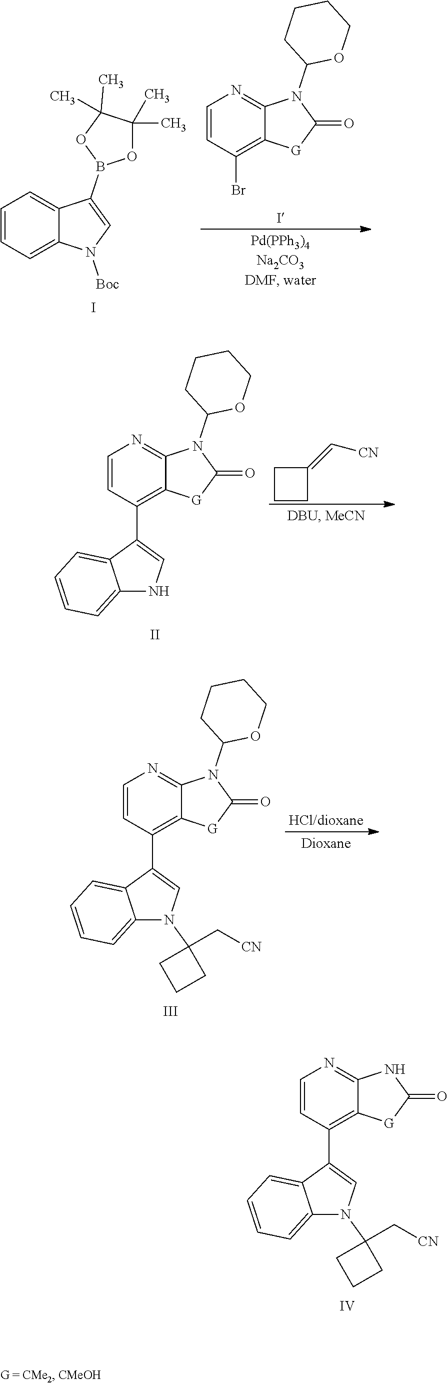

### 1. Suzuki Coupling

A reacti-vial was successively charged with bromine scaffold I′ (1.12 mmol), tert-butyl 3-(4,4,5,5-tetramethyl-1,3,2-dioxaborolan-2-yl)indole-1-carboxylate I (1.58 mmol, 1.4 eq.), disodium carbonate (3.35 mmol, 3 eq.), and tetrakistriphenylphosphine palladium (0.112 mmol, 0.1 eq.) in a mixture of DMF (9 mL) and water (1.9 mL). The vial was sealed, evacuated under vacuum and refilled with argon. The reaction was stirred at 100° C. overnight. The reaction mixture was diluted with EtOAc, filtered, washed with water, dried over Na₂SO₄ and evaporated. The crude material was purified by flash chromatography on silica gel using a gradient of heptane/EtOAc. Relevant fractions were collected and concentrated under vacuum to afford expected Suzuki coupling products II.

### Example: Synthesis of 4-(1H-indol-3-yl)-3,3-dimethyl-1-tetrahydropyran-2-yl-pyrrolo[2,3-b]pyridin-2-one (G=CMe₂)

Brown gum; yield 37%, ¹H NMR (DMSO-d₆, 400 MHz): δ (ppm) 11.47 (s, 1H), 8.21 (d, J=5.4 Hz, 1H), 7.51 (d, J=2.5 Hz, 1H), 7.48 (d, J=8.2 Hz, 1H), 7.30 (d, J=8.0 Hz, 1H), 7.22-7.11 (m, 1H), 7.06-6.97 (m, 2H), 5.48 (dd, J=11.3, 2.0 Hz, 1H), 3.99 (d, J=11.7 Hz, 1H), 3.65-3.51 (m, 1H), 3.08-2.87 (m, 1H), 1.95 (s, 1H), 1.71-1.45 (m, 4H), 1.14 (d, J=4.1 Hz, 6H); m/z=362.1 [M+H]+

### 2. Michael Reaction

A 4 mL reacti-vial was successively charged with cyclobutylideneacetonitrile (0.387 mmol, 2 eq.), Suzuki coupling products II (0.194 mmol, 1 eq.) in anhydrous acetonitrile (0.95 mL, 0.2N) and DBU (0.387 mmol, 2 eq.). The reaction was stirred at 85° C. overnight. Then 1 equivalent of cyclobutylideneacetonitrile was added and the reaction was stirred for 2 h. The reaction mixture was filtered and the precipitate was washed with acetonitrile to give afforded products III.

### Example: Synthesis of 2-[1-[3-(3,3-dimethyl-2-oxo-1-tetrahydropyran-2-yl-pyrrolo[2,3-b]pyridin-4-yl)indol-1-yl]cyclobutyl]acetonitrile (G=CMe₂)

White powder; yield 29%, ¹H NMR (DMSO-d₆, 400 MHz): δ (ppm) 8.22 (d, J=5.4 Hz, 1H), 7.46 (s, 1H), 7.43 (d, J=8.3 Hz, 1H), 7.33 (d, J=7.8 Hz, 1H), 7.22 (t, J=7.7 Hz, 1H), 7.11 (t, J=7.2 Hz, 1H), 6.99 (d, J=5.3 Hz, 1H), 5.52-5.41 (m, 1H), 3.99 (d, J=10.9 Hz, 1H), 3.58 (t, J=11.1 Hz, 1H), 3.48 (s, 2H), 2.95 (d, J=11.4 Hz, 1H), 2.76 (t, J=11.2 Hz, 2H), 2.63 (t, J=9.2 Hz, 2H), 2.28-2.12 (m, 1H), 1.97 (d, J=11.4 Hz, 2H), 1.73-1.40 (m, 4H), 1.16 (d, J=3.7 Hz, 6H); m/z=455.4 [M+H]+

### 3. Deprotection

4 M hydrogen chloride solution in dioxane (1.14 mmol, 20 eq.) was added to a solution of previous compounds III (0.057 mmol) in dioxane (0.2 mL, 0.3 N). The reaction was stirred at 45° C. overnight. Then 4 M hydrogen chloride solution in dioxane (1.14 mmol, 20 eq.) was added and the mixture stirred at 50° C. one more night. The solvent was evaporated. The crude material was purified via preparative HPLC under neutral conditions. Relevant fractions were combined and concentrated to give expected compounds IV.

### Example 21: Synthesis of 2-[1-[3-(3,3-dimethyl-2-oxo-1H-pyrrolo[2,3-b]pyridin-4-yl)indol-1-yl]cyclobutyl]acetonitrile (G=CMe₂)

White powder; yield 37%, ¹H NMR (DMSO-d₆, 600 MHz): δ (ppm) 11.04 (s, 1H), 8.09 (d, J=5.4 Hz, 1H), 7.43 (s, 1H), 7.42 (d, J=8.4 Hz, 1H), 7.34 (d, J=7.9 Hz, 1H), 7.21 (td, J=7.7, 1.0 Hz, 1H), 7.07-7.12 (m, 1H), 6.89 (d, J=5.3 Hz, 1H), 3.46 (s, 2H), 2.72-2.81 (m, 2H), 2.58-2.66 (m, 2H), 2.13-2.26 (m, 1H), 1.92-2.03 (m, 1H), 1.15 (s, 6H); m/z=371.0 [M+H]+.

### Scaffold Coupling—General Procedure (Indazole 1)

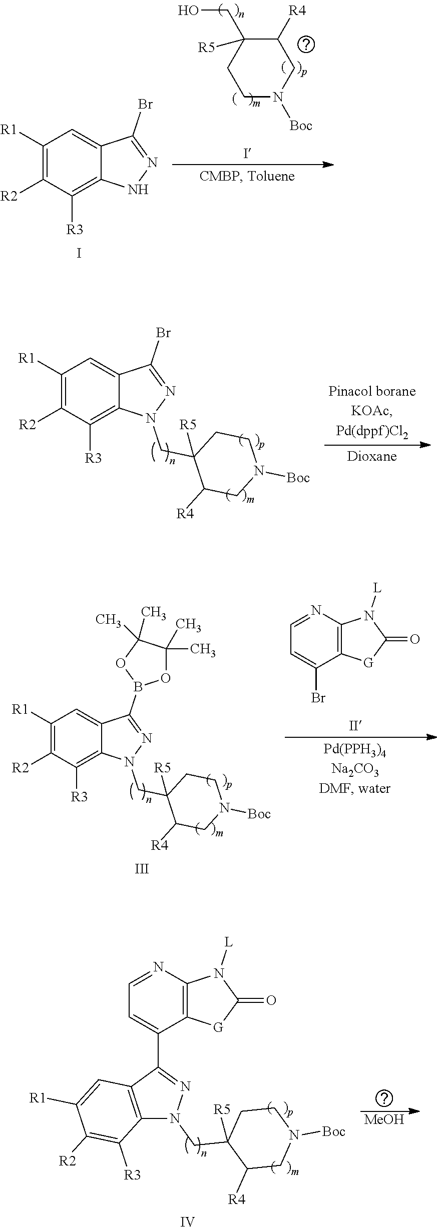

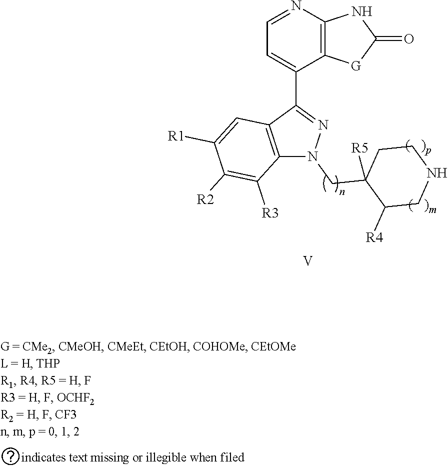

### 1. Mitsunobu Reaction

A sealed-vial was successively charged with bromoindazole I (1.23 mmol), anhydrous toluene (4 mL, 0.3 M), hydroxypiperidine-1-carboxylate I′ (2.46 mmol, 2 eq.) and cyanomethylenetributylphosphorane (2.46 mmol, 2 eq.) under nitrogen atmosphere. The reaction was stirred at 90° C. overnight. The solvent was evaporated to give crude material as a brown liquid. The crude material was purified by flash chromatography on silica gel using a gradient of heptane/ethyl acetate. It was transferred via solid pause on dicalite. Fractions were collected and concentrated under vacuum. A mixture of 2 diastereoisomers II was obtained. The mixture was used in the next step.

### Example: Synthesis of Tert-Butyl rac-(3R,4R)-4-(3-bromoindazole-1-yl)-3-fluoro-piperidine-1-carboxylate (R₁═R₂=R₃═R₅═H; R₄═F; n=0; m=p=1)

Yellow oil; diastereoisomers, quantitative yield, ¹H NMR (400 MHz, DMSO-d6) δ 7.81 (d, J=8.6 Hz, 1H), 7.60 (d, J=8.2 Hz, 1H), 7.57-7.50 (m, 1H), 7.32-7.26 (m, 1H), 5.09 (dd, J=18.2, 9.2 Hz, 1H), 4.90-4.68 (m, 1H), 4.35 (s, 1H), 4.09-4.01 (m, 1H), 3.06 (s, 2H), 2.07 (qd, J=10.0, 3.8 Hz, 2H), 1.45 (s, 9H); m/z=342.0, 344.0 [M-tBu+H]+

### 2. Boronic Ester Formation

In a 10 mL reacti-vial were introduced substituted bromoindazole II (0.753 mmol), bis(pinacolato)diboron (1.13 mmol, 1.5 eq) and bis(diphenylphosphino)ferrocene] dichloropalladium(II) (0.0753 mmol, 0.1 eq.) in anhydrous dioxane (2.5 mL, 0.3 N). The mixture was degassed with N₂ and stirred at 100° C. overnight. The mixture was filtered on dicalite and concentrated under vacuum to give crude material as a dark oil. The crude material Ill was used in next step without further purification.

### Example: Synthesis of Tert-Butyl rac-(3R,4R)-3-fluoro-4-[3-(4,4,5,5-tetramethyl-1,3,2-dioxaborolan-2-yl)indazol-1-yl]piperidine-1-carboxylate (R₁═R₂=R₃═R₅═H; R₄═F; n=0; m=p=1)

Black oil; m/z=364.0 [M+H]+ (boronic acid form)

### 3. Suzuki Coupling

A 10 mL vial was charged with bromine scaffold II′ (0.332 mmol), boronic esters III (0.602 mmol, 1.8 eq.) and disodium carbonate (0.996 mmol, 3 eq.) in a mixture of DMF (3 mL) and water (0.6 mL). The mixture was degassed and tetrakis triphenylphosphine palladium (0.033 mmol, 0.1 eq.) was added. The reaction was heated at 100° C. during 4 h. Water was added. The product precipitated and was filtered. It was then solubilized in DCM and the organic phase dried on phase separator and concentrated under vacuum. The crude material was purified by flash chromatography on silica gel using a gradient of heptane/EtOAc. Fractions were collected and concentrated under vacuum to afford expected Suzuki coupling compounds IV.

### Example: Synthesis of Tert-Butyl rac-(3R,4R)-4-[3-(3,3-dimethyl-2-oxo-1H-pyrrolo[2,3-b]pyridin-4-yl)indazol-1-yl]-3-fluoro-piperidine-1-carboxylate (R₁═R₂=R₃═R₅═H; R₄═F, n=0; m=p=1; G=CMe₂; L=H)

Beige powder; Yield 63%; ¹H NMR (DMSO-d₆, 500 MHz): δ (ppm) 11.13 (s, 1H), 8.24 (d, J=5.6 Hz, 1H), 7.80-7.90 (m, 2H), 7.47-7.57 (m, 1H), 7.36 (d, J=5.4 Hz, 1H), 7.23-7.31 (m, 1H), 5.20-5.36 (m, 1H), 4.71-4.98 (m, 1H), 4.26-4.49 (m, 1H), 4.03-4.12 (m, 1H), 2.94-3.23 (m, 2H), 2.03-2.27 (m, 2H), 1.44 (s, 9H), 1.34 (d, J=8.3 Hz, 6H); m/z=480.2 [M+H]+

### 4. Deprotection

4M hydrogen chloride in dioxane (1.04 mmol, 5 eq.) was added to a solution of Suzuki coupling products IV (0.21 mmol) in methanol (2 mL, 0.1 N). The mixture was stirred at room temperature overnight. The mixture was concentrated under vacuum. The product was triturated in diethyl ether and filtered. It was then dried under vacuum at 40° C. to afford final expected products V under salt forms.

### Racemate of Example 36: Synthesis of 3,3-dimethyl-4-[1-[rac-(3R,4R)-3-fluoro-4-piperidyl]indazol-3-yl]-1H-pyrrolo[2,3-b]pyridin-2-one;dihydrochloride (R₁═R₂=R₃═R₅═H; R₄═F, n=0; m=p=1; G=CMe₂)

Yellow powder; yield 91%, 1H NMR (500 MHz, DMSO-d6) δ ppm 11.16 (s, 1H); 8.97-10.01 (m, 2H), 8.24 (d, J=5.38 Hz, 1H); 7.84 (dd, J=8.31, 5.62 Hz, 2H), 7.50-7.59 (m, 1H), 7.34 (d, J=5.62 Hz, 1H), 7.27-7.32 (m, 1H), 5.37-5.48 (m, 1H), 5.19-5.37 (m, 1H); 3.85 (br s, 1H); 3.46-3.54 (m, 1H), 3.24-3.34 (m, 1H), 3.13-3.24 (m, 1H), 2.29-2.49 (m, 2H), 1.36 (d, J=13.20 Hz, 6H); m/z=380.0

### Scaffold Coupling—General Procedure (Indazole 2)

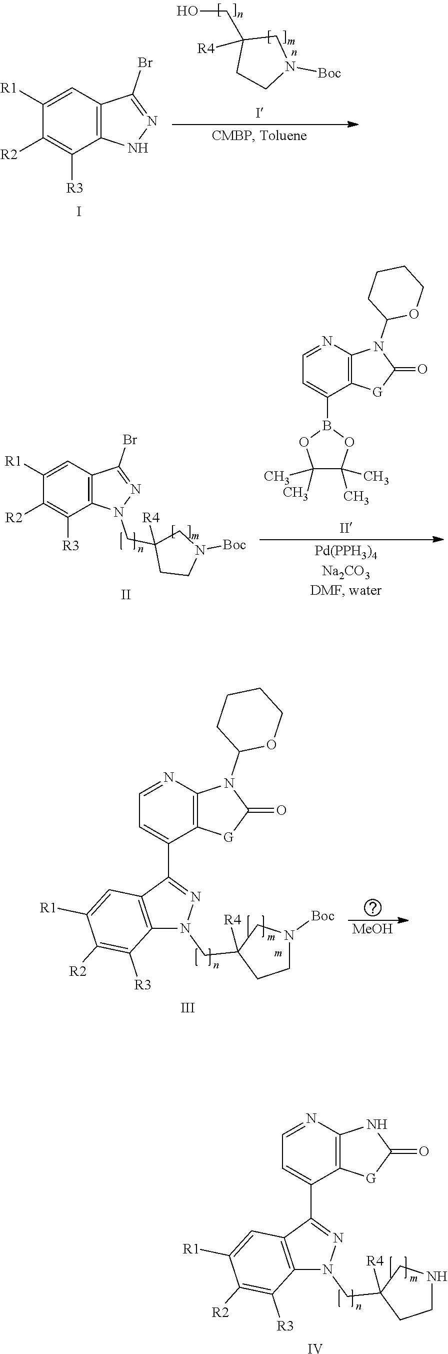

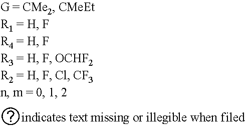

### 1. Mitsunobu

To a stirred mixture of bromoindazole I (1.97 mmol) in anhydrous toluene (8 mL, 0.25 N), were added cyanomethylenetributylphosphorane (5.91 mmol, 3 eq.) and hydroxypyrrolidine I′ (3.94 mmol, 2 eq.). The reaction was stirred at 100° C. overnight. The reaction mixture was concentrated to dryness and the crude was purified by flash chromatography column with a gradient of EtOAc in cyclohexane. Relevant fractions were collected and concentrated under vacuum to afford expected products II.

### Example: Synthesis of tert-butyl (3S)-3-(3-bromoindazole-1-yl)pyrrolidine-1-carboxylate (n=0, R₁═R₂═R₃=R₄═H)

Colorless oil, yield 44%, ¹H NMR (DMSO-d₆, 500 MHz): δ (ppm) 7.80 (d, J=8.6 Hz, 1H), 7.59 (d, J=8.1 Hz, 1H), 7.52 (ddd, J=8.4, 7.0, 1.0 Hz, 1H), 7.20-7.33 (m, 1H), 5.35-5.60 (m, 1H), 3.69-3.83 (m, 1H), 3.55 (dd, J=11.0, 4.9 Hz, 2H), 3.43 (br d, J=6.6 Hz, 1H), 2.20-2.45 (m, 2H), 1.32-1.50 (m, 9H); M/Z=366.2-368.2 [M+H]+

### 2. Suzuki Coupling

A 6-20 mL reacti-vial was successively charged with substituted bromoindazole II (0.41 mmol), disodium carbonate (1.23 mmol, 3 eq.), boronic ester II′ (0.491 mmol, 1.2 eq.) in a mixture of DMF (2.6 mL) and water (0.7 mL). The mixture was degassed and tetrakis triphenylphosphine palladium (0.0410 mmol, 0.01 eq.) was added. The reaction was stirred at 100° C. overnight. The reaction mixture was poured in water. The precipitate was filtered. The filtrate was solubilized with dichloromethane. The organic phase was dried on a phase separator and evaporated to give crude material. It was then purified by flash chromatography on silica gel using a gradient of heptane/EtOAc. Relevant fractions were collected and concentrated under vacuum to afford expected products III.

### Example: Synthesis of Tert-Butyl (3S)-3-[3-(3,3-dimethyl-2-oxo-1-tetrahydropyran-2-yl-pyrrolo[2,3-b]pyridin-4-yl)indazol-1-yl]pyrrolidine-1-carboxylate (n=0, R₁═R₂=R₃═R₄═H, G=CMe₂)

Beige foam; Yield 56%; 1H NMR (400 MHz, DMSO-d6) δ8.35 (d, J=5.4 Hz, 1H), 7.87 (dd, J=13.2, 8.4 Hz, 2H), 7.54 (ddd, J=8.3, 6.9, 0.9 Hz, 1H), 7.43 (d, J=5.4 Hz, 1H), 7.32-7.27 (m, 1H), 5.62 (s, 1H), 5.51-5.47 (m, 1H), 4.00 (d, J=11.3 Hz, 1H), 3.89 (s, 1H), 3.73 (d, J=10.6 Hz, 1H), 3.62-3.46 (m, 3H), 2.94 (d, J=11.4 Hz, 1H), 2.49-2.52 (m, 1H), 2.33 (s, 1H), 1.95 (s, 1H), 1.71-1.49 (m, 4H), 1.40 (d, J=5.2 Hz, 9H), 1.37 (s, 3H), 1.31 (d, J=3.6 Hz, 3H), m/z=532.4 [M+H]+

### 3. Deprotection

4 M hydrogen chloride solution in dioxane (9.18 mmol, 40 eq.) was added to a solution of Suzuki coupling compounds III (0.229 mmol) in anhydrous methanol (0.5 mL, 0.5 N). The reaction was stirred at 65° C. overnight. The reaction mixture was filtered to give final compounds IV (60 mg, 62% Yield) as hydrochlorhydric salts.

### Example 16: Synthesis of 3,3-dimethyl-4-[1-[(3S)-pyrrolidin-3-yl]indazol-3-yl]-1H-pyrrolo[2,3-b]pyridin-2-one Dihydrochloride (R₁═R₂=R₃═R₄═H, G=CMe₂)

White powder; yield 62%, 1H NMR (DMSO-d6, 500 MHz): δ (ppm) 11.14 (br s, 1H), 8.23 (d, J=5.4 Hz, 1H), 7.87 (d, J=8.6 Hz, 1H), 7.82 (d, J=8.3 Hz, 1H), 7.53 (t, J=7.7 Hz, 1H), 7.28 (q, J=4.8 Hz, 2H), 5.60 (s, 1H), 3.51-3.72 (m, 2H), 3.37 (br s, 4H), 2.44-2.48 (m, 1H), 2.33 (br d, J=5.9 Hz, 1H), 1.91 (s, 1H), 1.36 (s, 3H), 1.31 (s, 3H); m/z=348.0

### Scaffold Coupling—General Procedure (Indazole 3)

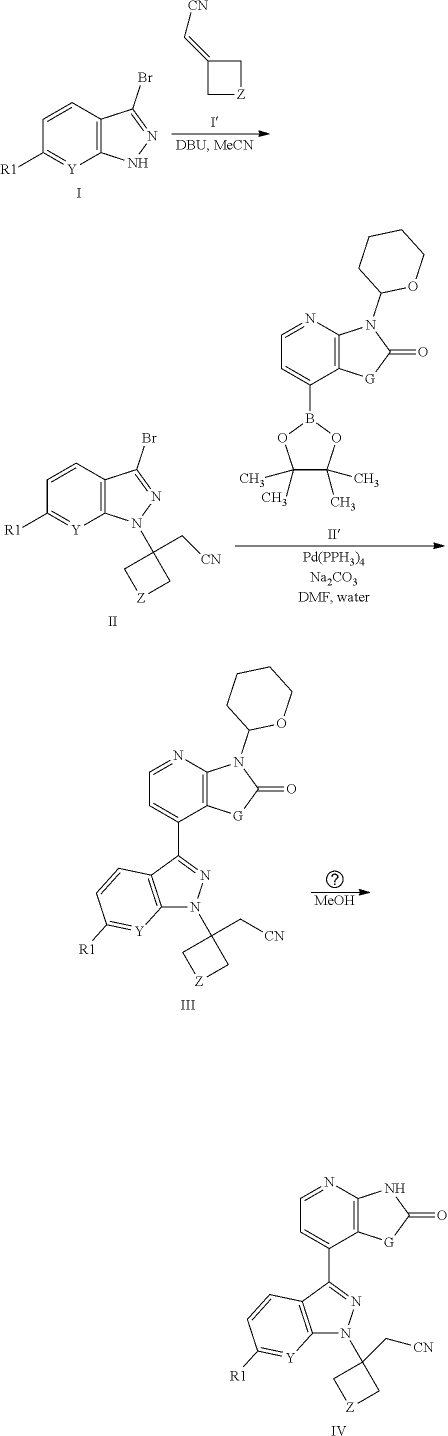

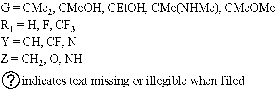

### 1. Michael Reaction

A 20 mL biotage vial was successively charged with 3-bromo-1H-indazole I (2.46 mmol), reactant I′ (2.46 mmol) and 1,8-diazabicyclo[5.4.0]undéc-7-ène (4.92 mmol, 2 eq.) in anhydrous acetonitrile (12 mL, 0.2 N). The mixture was stirred at 85° C. overnight. The mixture was concentrated under vacuum. The crude material was purified on flash chromatography with a gradient of Heptane/EtOAc. Relevant fractions were collected and concentrated under vacuum to afford corresponding products II.

### Example: Synthesis of 2-[1-(3-bromoindazole-1-yl)cyclobutyl]acetonitrile (Y═C—H, R₁═H, Z═CH₂)

White solid, yield 83%, ¹H NMR (DMSO-de, 500 MHz): δ (ppm) 7.60-7.64 (m, 2H), 7.47-7.52 (m, 1H), 7.26-7.32 (m, 1H), 3.38 (s, 2H), 2.80-2.94 (m, 2H), 2.52-2.61 (m, 2H), 2.17 (dquin, J=11.3, 9.0 Hz, 1H), 1.95 (dtt, J=11.2, 9.8, 3.2 Hz, 1H); m/z=290.1-292.1 [M+H]+

### 2. Suzuki Coupling

In an 20 mL biotage vial, substituted bromoindazole II (0.59 mmol, 1.1 éq.) was dissolved in a mixture of DMF (4 mL) and Water (1.3 mL), then boronic ester II′ (0.53 mmol) and disodium carbonate (1.60 mmol, 3 eq.) were added. The solution was degassed with N₂ and tetrakistriphenylphosphine palladium (0.053 mmol, 0.01 eq.) was added. The mixture was heated to 75° C. during 3 h. The solution was cooled and water was added. The product was extracted several times with EtOAc. Organic phases were gathered, filtered on a phase separator and concentrated under vacuum, to afford crude material. It was then purified by flash chromatography with a gradient of Heptane/EtOAc. Relevant fractions were collected and concentrated under vacuum to afford expected compounds III.

### Example: Synthesis of 2-[1-[3-(3-hydroxy-3-methyl-2-oxo-1-tetrahydropyran-2-yl-pyrrolo[2,3-b]pyridin-4-yl) indazol-1-yl]cyclobutyl]acetonitrile (Y═C—H, R₁═H, G=COHMe, Z═CH₂)

Pale yellow solid; Yield 15%; ¹H NMR (DMSO-d₆, 400 MHz): δ (ppm) 8.40 (dd, J=5.5, 0.9 Hz, 1H), 8.09 (d, J=8.4 Hz, 1H), 7.72 (d, J=8.6 Hz, 1H), 7.61 (dd, J=5.5, 2.0 Hz, 1H), 7.58-7.51 (m, 1H), 7.40-7.34 (m, 1H), 6.11 (d, J=5.3 Hz, 1H), 5.47 (d, J=11.2 Hz, 1H), 4.00 (d, J=8.7 Hz, 1H), 3.54-3.62 (m, 1H), 3.50 (s, 2H), 3.28-3.31 (m, 1H), 3.05-2.81 (m, 3H), 2.71-2.58 (m, 2H), 2.31-2.17 (m, 1H), 1.94-2.06 (d, J=1.8 Hz, 1H), 1.59 (d, J=50.3 Hz, 4H), 1.51 (d, J=1.4 Hz, 3H); m/z=458.4 [M+H]+

### 3. Deprotection

4 M hydrogen chloride solution in dioxane (1.5 mmol, 20 eq.) was added to a solution of Suzuki coupling compounds III (0.075 mmol) in anhydrous methanol (0.5 mL, 0.3N). The reaction was stirred at room temperature overnight then at 50° C. one night more. The mixture was basified with an aqueous solution of NaHCO₃, extracted with DCM, and the solvent was evaporated to give crude material. It was then purified in reverse phase with a gradient of H₂O/Acetonitrile. Fractions containing the product were gathered and concentrated to give expected products IV.

### Example 25: Synthesis of 2-[1-[3-(3-hydroxy-3-methyl-2-oxo-1H-pyrrolo[2,3-b]pyridin-4-yl)indazol-1-yl]cyclobutyl]acetonitrile (Y═C—H, R₁═H, G=COHMe, Z═CH₂)

White powder; yield 26%, ¹H NMR (DMSO-d₆, 500 MHz): δ (ppm) 11.09 (br s, 1H), 8.27 (d, J=5.4 Hz, 1H), 8.09 (d, J=8.3 Hz, 1H), 7.70 (d, J=8.6 Hz, 1H), 7.54 (ddd, J=8.4, 7.2, 1.0 Hz, 1H), 7.51 (d, J=5.6 Hz, 1H), 7.36 (dd, J=7.8, 6.8 Hz, 1H). 5.98 (s, 1H), 3.49 (d, J=2.2 Hz, 2H), 2.79-3.05 (m, 2H). 2.64 (ddd, J=12.2, 8.9, 2.7 Hz, 2H), 1.93-2.30 (m, 2H), 1.48 (s, 3H); m/z=374.2 [M+H]+

### Scaffold Coupling—General Procedure (Indazole 4)

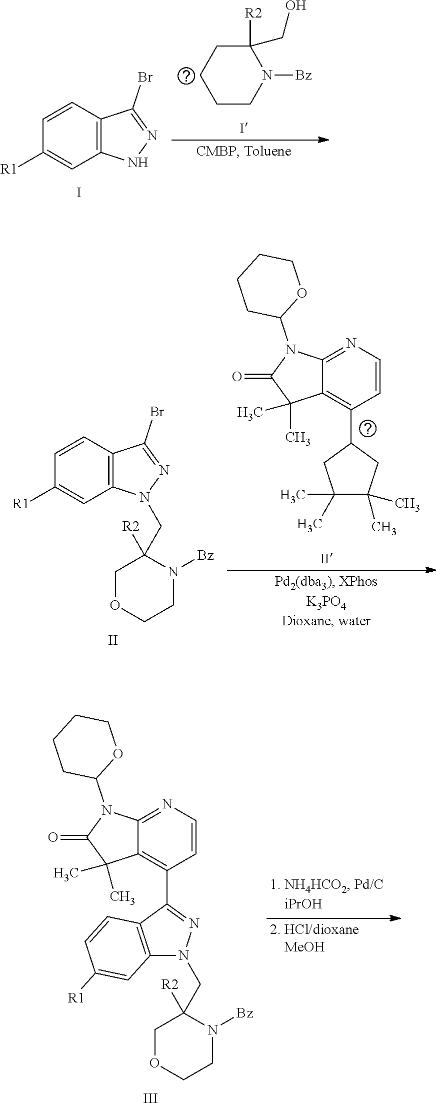

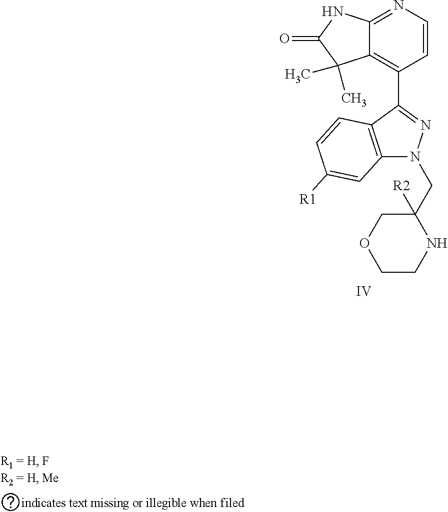

### 1. Mitsunobu

In a 10 mL vial, at room temperature, cyanomethylenetributylphosphorane (0.68 mL, 2.49 mmol, 2 eq.) was added to a stirred solution of bromoindazole I (1.24 mmol, 1 eq.) and alcohol (1.24 mmol, 1 eq.) in anhydrous toluene (3.7 mL, 0.3 N). The reaction mixture was stirred at 80° C. for 5 h. The reaction was allowed to reach room temperature, concentrated under vacuum to give crude material as a brown oil. The crude material was purified by flash chromatography on silica gel using a gradient of heptane/EtOAc. It was transferred via liquid injection in DCM. Relevant fractions were collected and concentrated under vacuum to give expected compounds II.

### Example: Synthesis of 4-benzyl-3-[(3-bromoindazole-2-yl)methyl]-3-methyl-morpholine (R₁═H, R₂=Me) 30

Colorless gum, 45% yield, 1H NMR (400 MHz, DMSO-d6) δ 7.82 (d, J=8.6 Hz, 1H), 7.60 (d, J=8.2 Hz, 1H), 7.54-7.41 (m, 1H), 7.40-7.16 (m, 6H). 4.83 (d, J=14.7 Hz, 1H), 4.54 (d, J=14.7 Hz, 1H), 3.93-3.79 (m, 1H), 3.75 (d, J=13.9 Hz, 1H), 3.63 (dd, J=9.8, 5.0 Hz, 1H), 3.51 (ddd, J=11.2, 8.2, 3.2 Hz, 1H), 3.43 (d, J=11.3 Hz, 1H), 3.26 (d, J=11.3 Hz, 1H), 2.69 (ddd, J=11.7, 8.2, 3.3 Hz, 1H), 2.43-2.28 (m, 1H), 1.06 (s, 3H); m/z=400.3, 402.3[M+H]+.

### 2. Suzuki Coupling

A 6-20 mL reacti-vial was successively charged with substituted bromoindazole II (0.532 mmol), 3,3-dimethyl-1-tetrahydropyran-2-yl-4-(4,4,5,5-tetramethyl-1,3,2-dioxaborolan-2-yl)pyrrolo[2,3-b]pyridin-2-one (0.532 mmol, 1 eq.), tripotassium phosphate (229 mg, 1.06 mmol, 3 eq.), Xphos (10 mg, 0.021 mmol, 0.04 eq.), tris(dibenzylideneacetone)dipalladium(0) (0.011 mmol, 0.02 eq.), dioxane (2.2 mL) and water (0.4 mL). The vial was sealed, evacuated under vacuum and refilled with argon. The reaction was stirred at 95° C. overnight. Water and EtOAc were added. The two phases were separated and the aqueous phase was extracted with EtOAc. Combined organic phases were dried using a phase separator and evaporated to give crude material. The crude material was purified by flash chromatography on silica gel using a gradient of heptane/EtOAc. It was transferred via liquid injection in DCM. Relevant fractions were collected and concentrated under vacuum to give expected products III.

### Example: Synthesis of 4-[1-[(4-benzyl-3-methyl-morpholin-3-yl)methyl]indazol-3-yl]-3,3-dimethyl-1-tetrahydropyran-2-yl-pyrrolo[2,3-b]pyridin-2-one (R₁═H, R₂=Me)

White foam; 67% yield; ¹H NMR (500 MHz, DMSO-d₆) δ 8.34 (d, J=5.38 Hz, 1H), 7.90 (d, J=8.56 Hz, 1H), 7.79 (d, J=8.31 Hz, 1H), 7.50 (t, J=7.61 Hz, 1H), 7.39 (d, J=5.38 Hz, 1H), 7.19-7.32 (m, 6H), 5.47-5.50 (m, 1H), 4.95 (d, J=14.43 Hz, 1H), 4.65 (d, J=14.67 Hz, 1H), 3.91-4.01 (m, 2H), 3.68-3.78 (m, 2H), 3.51-3.64 (m, 3H), 3.26-3.29 (m, 1H), 2.90-2.99 (m, 1H), 2.75 (br t, J=8.80 Hz, 1H), 2.37-2.42 (m, 1H), 1.96 (br d, J=11.49 Hz, 1H), 1.49-1.69 (m, 4H), 1.31-1.39 (m, 6H), 1.11 (s, 3H); m/z=566.5 [M+H]+

### 3. Deprotection of Benzyl

Suzuki coupling product III (0.09 mmol) was dissolved in anhydrous methanol (2.2 mL, 0.04 N). Ammonium formate (0.62 mmol, 7 eq.) and Pd/C 10% Engelhard (0.09 mmol, 1 eq.) were added. The reacti-vial was sealed, evacuated under vacuum and refilled with argon. The suspension was stirred at 110° C. for 3 h. The reaction mixture was filtered, washed with MeOH and concentrated, to give benzyl deprotected products.

### Example: Synthesis of 3,3-dimethyl-4-[1-[(3-methylmorpholin-3-yl)methyl]indazol-3-yl]-1-tetrahydropyran-2-yl-pyrrolo[2,3-b]pyridin-2-one (R₁═H, R₂=Me)

Brown gum; 61% yield; ¹H NMR (600 MHz, DMSO-D6, 300 K) δ (ppm)=8.33 (d, J=5.3 Hz, 1H), 7.84 (d, J=8.5 Hz, 1H), 7.78 (d, J=8.2 Hz, 1H), 7.50 (ddd, J=0.9, 7.1, 8.3 Hz, 1H), 7.38 (d, J=5.4 Hz, 1H), 7.27-7.20 (m, 1H), 5.49 (dd, J=2.1, 11.4 Hz, 1H), 4.69 (d, J=14.5 Hz, 1H), 4.54 (d, J=14.5 Hz, 1H), 3.99 (td, J=1.9, 11.4 Hz, 1H). 3.65 (td, J=3.8, 11.0 Hz, 1H), 3.61-3.46 (m, 3H), 3.13-3.06 (m, 1H), 3.04-2.83 (m, 1H), 2.78-2.70 (m, 1H), 2.37-2.14 (m, 1H), 1.96 (br dd, J=2.5, 10.0 Hz, 1H), 1.72-1.44 (m, 5H), 1.37 (d, J=1.0 Hz, 6H), 0.94 (s, 3H); m/z=476.5 [M+H]+

### 4. Deprotection of THP

4 M hydrogen chloride solution in dioxane (8 mmol, 8 eq.) was added to a solution of previous compounds (0.17 mmol) in anhydrous methanol (0.8 mL, 0.2 N). The reaction was stirred at 65° C. overnight. After cooling, an aqueous solution of NaHCO₃ was added and the mixture was extracted with EtOAc. Combined organic phases were dried over Na₂SO₄, filtered and evaporated to give expected compounds IV as hydrochloric salts.

### Racemic precursor of Example 31: Synthesis of 3,3-dimethyl-4-[1-[(3-methylmorpholin-3-yl)methyl]indazol-3-yl]-1H-pyrrolo[2,3-b]pyridin-2-one (R₁═H, R₂=Me)

White powder; yield 62%, 1H NMR (DMSO-d6, 600 MHz): δ (ppm) 11.10 (s, 1H), 8.21 (d, J=5.4 Hz, 1H), 7.83 (d, J=8.7 Hz, 1H), 7.80 (d, J=8.2 Hz, 1H), 7.49 (ddd, J=8.3, 7.0, 1.0 Hz, 1H), 7.29 (d, J=5.4 Hz, 1H), 7.22-7.26 (m, 1H), 4.69 (d, J=14.5 Hz, 1H), 4.53 (d, J=14.5 Hz, 1H), 3.65 (dt, J=10.8, 3.8 Hz, 1H), 3.58 (d, J=11.2 Hz, 1H), 3.49 (ddd, J=10.8, 8.3, 2.9 Hz, 1H), 3.28-3.30 (m, 1H), 3.10 (ddd, J=12.3, 8.6, 3.2 Hz, 1H), 2.71-2.78 (m, 1H), 2.33 (br s, 1H), 1.36 (d, J=1.6 Hz, 6H), 0.94 (s, 3H); m/z=392.1 [M+H]+

Enantiomeric Compounds were Obtained after Chiral Purification.

### Example 63 was Obtained as a Secondary Product During Benzyl Deprotection in Methanol

### Scaffold Coupling—General Procedure (Indazole 5)

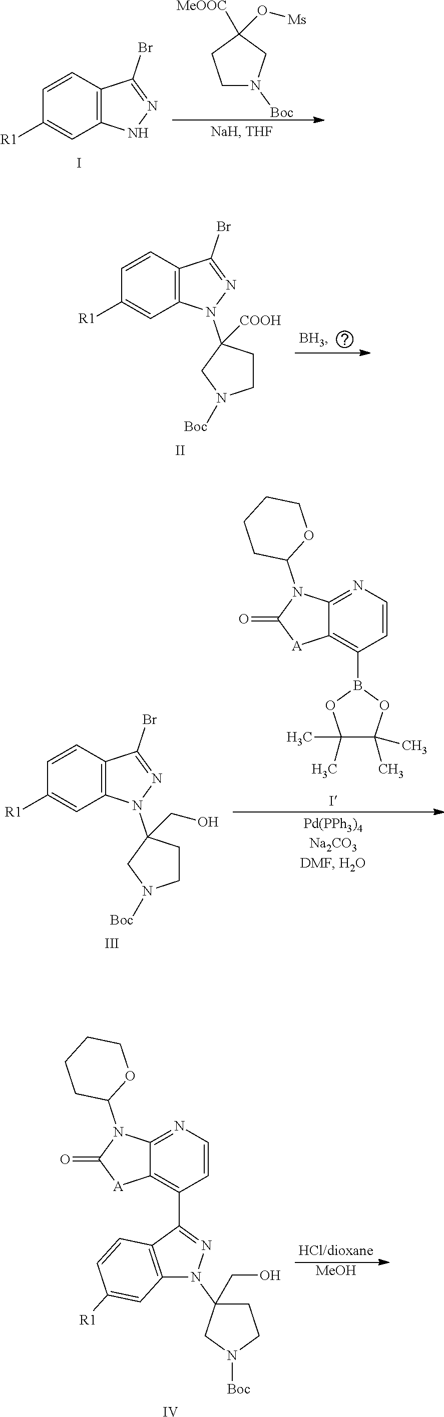

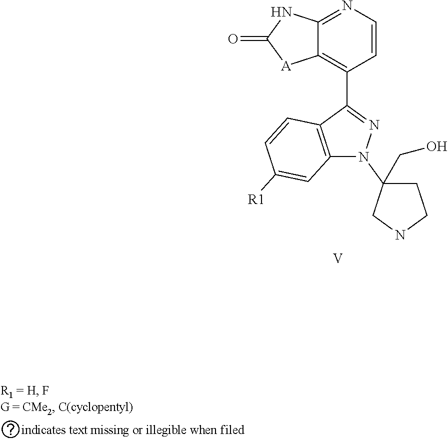

1. Synthesis of O1-tert-butyl O3-methyl 3-methylsulfonyloxypyrrolidine-1,3-dicarboxylate

At room temperature, methanesulfonyl chloride (1.2 mL, 14.9 mmol, 2 eq.) was added to a stirred suspension of triethylamine (2.1 mL, 14.9 mmol, 2 eq.) and 1-tert-butyl 3-methyl 3-hydroxypyrrolidine-1,3-dicarboxylate (1.90 g, 7.44 mmol, 2 eq.) in anhydrous dichloroethane (62 mL, 0.12 N). The reaction was stirred at 55° C. overnight. Water was added and the mixture extracted with DCE. The organic phase was dried and concentrated under vacuum. The crude was purified by flash chromatography using a gradient of heptane/EtOAc. Relevant fractions were collected and concentrated under vacuum to afford O1-tert-butyl O3-methyl 3-methylsulfonyloxypyrrolidine-1,3-dicarboxylate (1.5 g, 63% Yield) as a yellow oil. 1H NMR (Chloroform-d, 400 MHz): δ (ppm) 3.92 (t, J=14.9 Hz, 5H), 3.69-3.49 (m, 2H), 3.18 (s, 3H), 2.66-2.41 (m, 2H), 1.46 (s, 9H).

### 2. Substitution

A reacti-vial was charged with sodium hydride (1.99 mmol, 1.5 eq.) and anhydrous THF (0.05 mL). Then bromoindazole I (300 mg, 1.33 mmol) in anhydrous THF (0.1 mL) was added. O1-tert-butyl O3-methyl 3-methylsulfonyloxypyrrolidine-1,3-dicarboxylate (1.99 mmol, 2 eq.) in anhydrous THF (0.35 mL) was added dropwise at room temperature. The reaction was stirred at room temperature overnight. Sodium hydride (1.5 eq) was added and the reaction was stirred overnight at room temperature. The solvent was evaporated. This residue was solubilized in ethyl acetate and water was added. The two phases were separated. Combined aqueous phases were neutralized with an aqueous solution of HCl 1N and extracted with dichloromethane. Combined organic phases were dried using a phase separator and evaporated to give afforded products II.

### Example: Synthesis of 3-(3-bromo-6-fluoro-indazol-1-yl)-1-tert-butoxycarbonyl-pyrrolidine-3-carboxylic Acid (R₁═F)

Colorless gum, 37% yield, 1H NMR (400 MHz, DMSO-d6) δ 13.72 (s, 1H), 7.69 (dd, J=8.9, 5.2 Hz, 1H), 7.46 (s, 1H), 7.26-7.17 (m, 1H), 4.37-4.10 (m, 2H), 3.02-2.83 (m, 2H), 2.42 (s, 1H), 2.15 (s, 1H), 1.42 (s, 9H); m/z=400.3, 402.3 [M+H]+.

### 3. Reduction

A 2 mL reacti-vial was charged with substituted indazoles II (0.487 mmol) and a solution of 1 M borane tetrahydrofuran (0.974 mmol, 2 eq.) was added. The reaction was stirred at room temperature for 3 h. The reaction mixture was poured in an aqueous solution of NH₄Cl sat. and extracted with dichloromethane. Combined organic phases were dried over phase separator and evaporated to give crude material as a brown solid. The crude material was purified by flash chromatography on silica gel using a gradient of dichloromethane/ethyl acetate. It was transferred via solid pause on Isolute HM-N. Relevant fractions were collected and concentrated under vacuum to afford expected products III.

### Example: Synthesis of Tert-Butyl 3-(3-bromo-6-fluoro-indazol-1-yl)-3-(hydroxymethyl)pyrrolidine-1-carboxylate (R₁═F)

White foam; 44% yield; m/z=358, 360 [M+H-tBu]+

### 4. Suzuki

A 12 mL reacti-vial was charged with previous compounds III (0.222 mmol), boronic ester I′ (0.222 mmol, 1 eq.) and tetrakistriphenylphosphine palladium (0.0222 mmol, 0.1 eq.) in a mixture of DMF (1.7 mL) and Water (0.6 mL). The mixture was degassed with N₂, then disodium carbonate (0.666 mmol, 3 eq.) was added. The mixture was once again degassed with N₂, then it is stirred at 90° C. for 3 h. Water was added to the cold mixture and the precipitate was filtered and washed with water, then dissolved in DCM. The organic phase was filtered on a phase separator and concentrated under vacuum. The crude product was purified with a gradient of heptane/EtOAc, Relevant fractions were collected and concentrated under vacuum to afford expected compounds IV.

### Example: Synthesis of Tert-Butyl 3-[6-fluoro-3-(2′-oxo-1′-tetrahydropyran-2-yl-spiro[cyclopentane-1,3′-pyrrolo[2,3-b]pyridine]-4′-yl)indazol-1-yl]-3-(hydroxymethyl)pyrrolidine-1-carboxylate (R₁═F, G=CCyclopentyl)

Beige solid, 24% yield, m/z=606.3 [M+H]+

### 5. Deprotection

4 M hydrogen chloride solution in dioxane (1.9 mmol, 40 eq.) was added to a solution of previous compounds IV (0.05 mmol) in anhydrous methanol (0.1 mL, 0.5 N). The reaction was stirred at 65° C. overnight. The solution was concentrated. The product was dissolved in water and the impurities were extracted with EtOAc. The aqueous phase was concentrated under vacuum to afford expected compounds IV as hydrochloric salts.

### Example 55: Synthesis of 4-[6-fluoro-1-[3-(hydroxymethyl)pyrrolidin-3-yl]indazol-3-yl]spiro[1H-pyrrolo[2,3-b]pyridine-3,1′-cyclopentane]-2-one dihydrochloride (R₁═F, G=CCyclopentyl)

Yellow powder; 57% yield; 1H NMR (DMSO-d6, 500 MHz): δ (ppm) 11.02 (s, 1H), 9.18-9.66 (m, 2H), 8.19 (d, J=5.4 Hz, 1H), 7.65-7.84 (m, 2H), 7.17 (td, J=9.0, 2.1 Hz, 1H), 7.11 (d, J=5.4 Hz, 1H), 4.10 (dt, J=12.0, 6.0 Hz, 2H), 3.80-3.97 (m, 3H), 3.40-3.52 (m, 1H), 3.23-3.34 (m, 1H), 2.66-2.93 (m, 2H), 2.10-2.28 (m, 2H), 1.77-1.90 (m, 4H), 1.60 (br d, J=2.7 Hz, 2H); m/z=422.1 [M+H]+

### In Order to Obtain Example 77 One More Step Will be Performed Before Deprotection

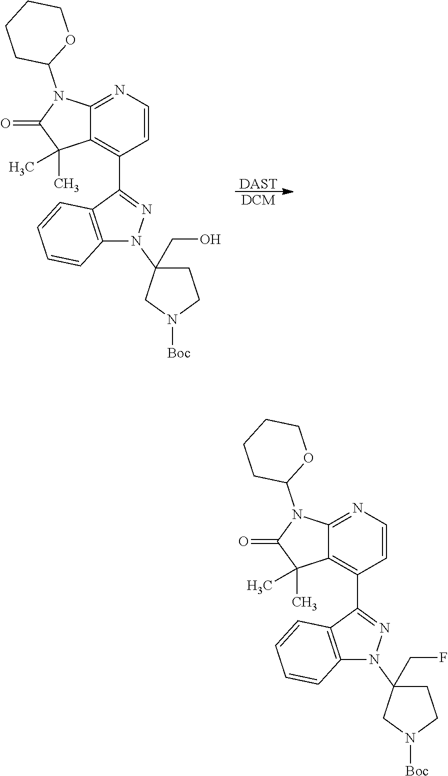

### Synthesis of tert-butyl 3-[3-(3,3-dimethyl-2-oxo-1-tetrahydropyran-2-yl-pyrrolo[2,3-b]pyridin-4-yl)indazol-1-yl]-3-(fluoromethyl)pyrrolidine-1-carboxylate

In a 5 ml reacti-vial at 0° C., DAST (0.019 mL, 0.142 mmol, 1.5 eq.) was added dropwise to a stirred solution of tert-butyl 3-[3-(3,3-dimethyl-2-oxo-1-tetrahydropyran-2-yl-pyrrolo[2,3-b]pyridin-4-yl)indazol-1-yl]-3-(hydroxymethyl)pyrrolidine-1-carboxylate (53 mg, 0.0944 mmol) in anhydrous DCM (1.2 mL, 0.08 N). The reaction mixture was allowed to reach room temperature and stirred at room temperature overnight. DAST (0.019 mL, 0.142 mmol, 3 eq.) was added and the reaction mixture was stirred at room temperature over weekend. The reaction mixture was quenched with an aqueous solution of NaOH 1M until pH=12, extracted with dichloromethane and dried over phase separator. The solvent was evaporated to give tert-butyl 3-[3-(3,3-dimethyl-2-oxo-1-tetrahydropyran-2-yl-pyrrolo[2,3-b]pyridin-4-yl)indazol-1-yl]-3-(fluoromethyl)pyrrolidine-1-carboxylate (35.3 mg. 66% Yield) as a pale yellow gum. It was engaged in next step without further purification. ¹H NMR (400 MHz, DMSO-d₆) δ 8.36 (d, J=5.4 Hz, 1H), 7.94-7.77 (m, 2H), 7.58-7.48 (m, 1H), 7.41 (d, J=6.3 Hz, 1H), 7.37-7.27 (m, 1H). 5.49 (d, J=11.1 Hz, 1H), 5.10-4.78 (m, 2H), 4.09-3.95 (m, 1H), 3.57 (d, J=10.2 Hz, 2H), 2.97 (dq, J=13.7, 6.9 Hz, 3H), 2.75 (q, J=7.2 Hz, 3H), 1.95 (s, 1H), 1.71-1.45 (m, 4H), 1.38 (d, J=12.7 Hz, 6H), 1.07 (dt, J=20.1, 7.2 Hz, 9H); m/z=564.2 [M+H]+.

### Scaffold Coupling—Specific Procedure (Specific Indazole 1)

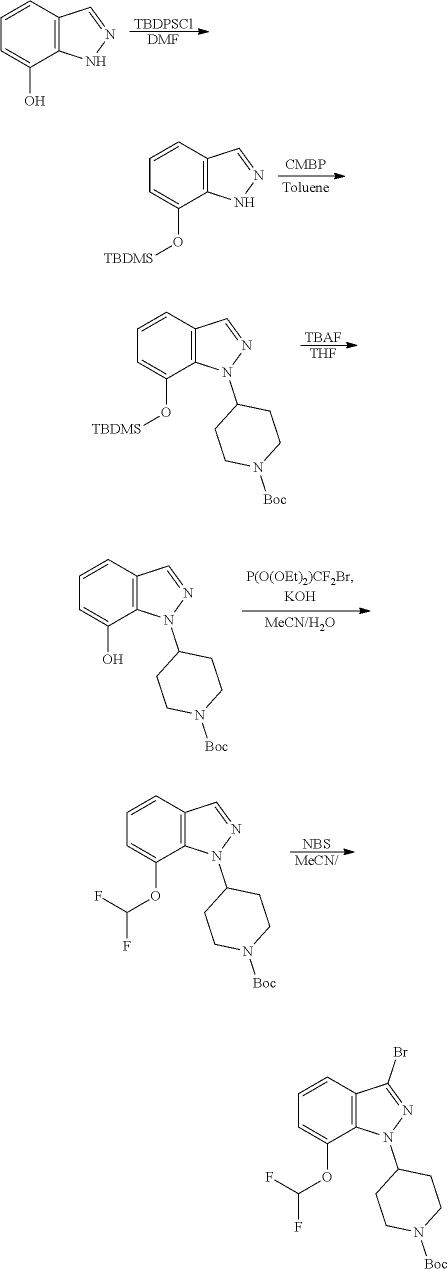

### Step 1: Synthesis of tert-butyl-(1H-indazol-7-yloxy)-diphenyl-silane

To a solution of 7-hydroxy-1H-indazole (95%, 1.44 g, 10.2 mmol) in anhydrous DMF (14 mL, 0.5 N) was added tert-butylchlorodiphenylsilane (6.8 mL, 25.5 mmol, 2.5 eq.) at room temperature. The resulting mixture was stirred at room temperature overnight. tert-butyl-chlorodiphenylsilane (6.8 mL, 25.5 mmol, 2.5 eq.) was added and the resulting mixture was stirred at 80° C. overnight. The reaction was poured into an aqueous solution of NaHCO₃ and extracted with EtOAc. The two phases were separated and the organic phase was washed with water, dried over Na₂SO₄, filtered and evaporated. The crude material was purified by flash chromatography on silica gel using a gradient of heptane/EtOAc. It was transferred via liquid injection in DCM. Relevant fractions were collected and concentrated under vacuum to afford tert-butyl-(1H-indazol-7-yloxy)-diphenyl-silane (1.4 g, 37% yield) as a white foam. ¹H NMR (DMSO-d₆, 500 MHz): δ (ppm) 13.36 (s, 1H), 8.07 (s, 1H), 7.71-7.74 (m, 4H), 7.40-7.51 (m, 6H), 7.23 (d, J=8.1 Hz, 1H), 6.63 (t, J=7.8 Hz, 1H), 6.13 (d, J=7.3 Hz, 1H), 1.09 (s, 9H); m/z=373.4 [M+H]+

### Step 2: Synthesis of 4-[7-[tert-butyl(diphenyl)silyl]oxydiazol-1-yl]piperidine-1-carboxylate

To a stirred mixture of tert-butyl-(1H-indazol-7-yloxy)-diphenyl-silane (650 mg, 1.74 mmol) in anhydrous toluene (5 mL, 0.3 N), were added cyanomethylenetributylphosphorane (0.91 mL, 3.49 mmol, 2 eq.) and tert-butyl 4-hydroxypiperidine-1-carboxylate (0.70 g, 3.49 mmol, 2 eq.). The reaction was stirred at 85° C. during 5 h. tert-butyl 4-hydroxypiperidine-1-carboxylate (0.70 g, 3.49 mmol, 2 eq.) and cyanomethylenetributylphosphorane (0.91 mL, 3.49 mmol, 2 eq.) were added again and the reaction was stirred at 85° C. overnight. The reaction mixture was concentrated to dryness and the crude was purified by flash chromatography column with a gradient of EtOAc in Cyclohexane. Relevant fractions were collected and concentrated under vacuum to afford tert-butyl 4-[7-[tert-butyl(diphenyl)silyl]oxyindazol-1-yl]piperidine-1-carboxylate (180 mg, 18%). 1H NMR (600 MHz, DMSO-d6) Shift 8.09 (s, 1H), 7.71-7.76 (m, 4H), 7.38-7.52 (m, 6H), 7.23 (d, J=8.02 Hz, 1H), 6.64 (t, J=7.85 Hz, 1H), 6.21 (d, J=7.63 Hz, 1H), 5.57 (tt, J=5.28, 10.27 Hz, 1H), 4.14 (br d, J=12.03 Hz, 2H), 2.78 (br s, 2H), 2.02-2.11 (m, 4H), 1.38-1.45 (m, 9H), 1.11 (s, 9H); m/z=556.4 [M+H]+.

### Step 3: Synthesis of tert-butyl 4-(7-hydroxyindazol-1-yl)piperidine-1-carboxylate

In a 50 mL round-bottomed flask, at room temperature, 1 M tetrabutylammonium fluoride solution (0.49 mL, 0.486 mmol, 1.5 eq.) was added to a stirred solution of tert-butyl 4-[7-[tert-butyl(diphenyl)silyl]oxyindazol-1-yl]piperidine-1-carboxylate (180 mg, 0.324 mmol) in anhydrous THF (1.6 mL, 0.2 N). The reaction was stirred at room temperature for 1 h. The reaction was quenched with brine, and EtOAc was added. The two phases were separated and the organic phase was washed with water and brine, dried over Na₂SO₄, and evaporated to dryness. The crude material was triturated in DCM. The solid was filtered and dried under high vacuum to give tert-butyl 4-(7-hydroxyindazol-1-yl)piperidine-1-carboxylate (70 mg, 68% Yield) as a white solid. 1H NMR (400 MHz, DMSO-d6) δ 10.20 (s, 1H), 7.95 (s, 1H), 7.14 (dd, J=8.0, 0.6 Hz, 1H), 6.96-6.82 (m, 1H), 6.69 (dd, J=7.4, 0.6 Hz, 1H), 5.25 (p, J=7.9, 7.4 Hz, 1H), 4.09 (d, J=11.7 Hz, 2H), 2.93 (s, 2H), 2.02-1.87 (m, 4H), 1.43 (s, 9H); m/z=318.1 [M+H]+

### Step 4: Synthesis of Tert-Butyl 4-[7-(difluoromethoxy)indazol-1-yl]piperidine-1-carboxylate

In a 2-5 mL sealed tube, at −78° C., diethyl [bromo(difluoro)methyl]phosphonate (0.080 mL, 0.429 mmol, 2 eq.) was added in one portion to a cooled solution of tert-butyl 4-(7-hydroxyindazol-1-yl)piperidine-1-carboxylate (68 mg, 0.214 mmol) and potassium hydroxide (240 mg, 4.29 mmol, 20 eq.) in a mixture of acetonitrile (1.1 mL) and water (1.1 mL). The reaction was allowed to warm to room temperature and stirred during 1 h. The reaction mixture was diluted with EtOAc. The two phases were separated and the aqueous phase was extracted with EtOAc. Combined organic phases were washed with brine, water, dried over Na₂SO₄, and evaporated to give tert-butyl 4-[7-(difluoromethoxy)indazol-1-yl]piperidine-1-carboxylate as a brown gum. ¹H NMR (600 MHz, DMSO-d₆) δ ppm 8.17 (s, 1H), 7.63-7.67 (m, 1H), 7.27-7.53 (m, 1H), 7.16-7.19 (m, 1H), 7.11-7.15 (m, 1H), 4.95-5.04 (m, 1H), 4.03-4.18 (m, 2H), 2.71-3.11 (m, 2H), 1.83-2.06 (m, 4H), 1.43 (s, 9H); m/z=312.2 [M+H]+

### Step 5: Synthesis of tert-butyl 4-[3-bromo-7-(difluoromethoxy)indazol-1-yl]piperidine-1-carboxylate

N-bromosuccinimide (23 mg, 0.128 mmol, 1.05 eq.) was added to a solution of tert-butyl 4-[7-(difluoromethoxy)indazol-1-yl]piperidine-1-carboxylate (64 mg, 0.122 mmol) in acetonitrile (0.3 mL, 0.4 N). The mixture was stirred at room temperature overnight. The solvent was removed under vacuum, and the residue was dissolved in EtOAc. The organic solution was washed with an aqueous solution of NaOH, water, dried over Na₂SO₄, and concentrated under vacuum to give tert-butyl 4-[3-bromo-7-(difluoromethoxy)indazol-1-yl]piperidine-1-carboxylate (69 mg, 85% yield) as a purple gum. ¹H NMR (DMSO-d₆, 500 MHz): δ (ppm) 7.15-7.71 (m, 4H), 4.89-5.07 (m, 1H), 3.99-4.19 (m, 2H), 2.75-3.10 (m, 2H), 1.72-2.15 (m, 4H), 1.36-1.46 (m, 9H); m/z=390.2, 392.2 [M+H-tBu]+

The next steps were similar to general procedure—indazole 2.

### Scaffold Coupling—Specific Procedure (Specific Indazole 2)

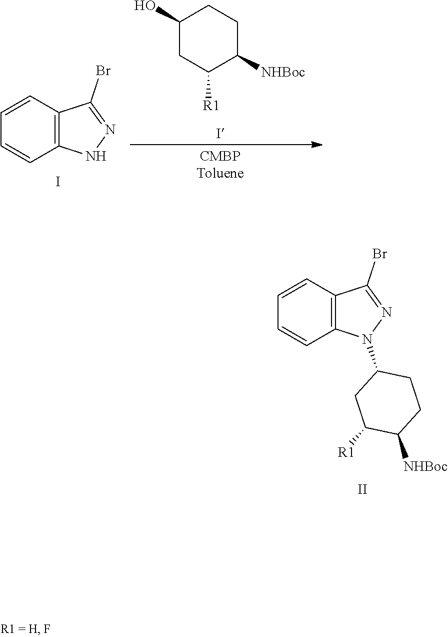

A 6 ml sealed-vial was successively charged with 3-bromo-1H-indazole (120 mg, 0.59 mmol), (tributyl-lambda˜5-phosphanylidene)acetonitrile (0.31 mL, 1.18 mmol) and compound I′ in anhydrous toluene (2 mL) under nitrogen atmosphere. The reaction was stirred at 80° C. overnight. The solvent was evaporated to give crude material. It was purified by flash chromatography on silica gel using a gradient of heptane/ethyl acetate. It was transferred via solid pause on Isolute HM-N on a 12 g Redisep Gold column. Relevant fractions were collected and concentrated under vacuum to afford compounds II.

### Example: Synthesis of Tert-Butyl N-[rac-(1R,2R,4R)-4-(3-bromoindazol-1-yl)-2-fluoro-cyclohexyl]carbamate (R₁═F)

Beige solid, 58% yield, 1H NMR (400 MHz, DMSO-d6) δ 7.88 (d, J=8.6 Hz, 1H), 7.58 (d, J=8.2 Hz, 1H), 7.29-7.23 (m, 1H), 7.13-7.03 (m, 1H), 4.81 (s, 1H), 4.63 (s, 1H), 4.50 (s, 1H), 3.57 (s, 1H), 2.42 (d, J=5.2 Hz, 1H), 2.21-2.08 (m, 1H), 1.91 (d, J=7.3 Hz, 2H), 1.45 (d, J=7.0 Hz, 2H), 1.42 (s, 9H).

### Scaffold Coupling—Specific Procedure (Specific Indazole 3)

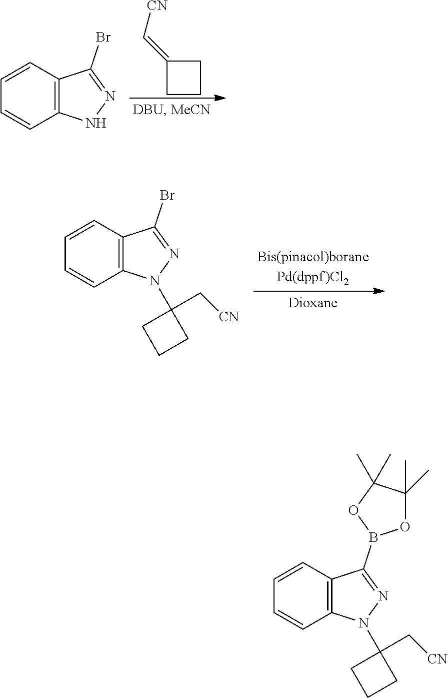

### Step 1: Synthesis of 2-[1-(3-bromoindazol-1-yl)cyclobutyl]acetonitrile

A 20 ml biotage vial was successively charged with 3-bromo-1H-indazole (500 mg, 2.46 mmol), cyclobutylideneacetonitrile (0.25 mL, 2.46 mmol), 1,8-diazabicyclo[5.4.0]undéc-7-ene (0.73 mL, 4.92 mmol) in anhydrous acetonitrile (12 mL). The mixture was stirred at 85° C. during 48 h. The mixture was concentrated under vacuum. An orange oil was obtained and purified on a 12 g silica gel column with a gradient of Heptane/EtOAc. Relevant fractions were collected and concentrated under vacuum to afford 2-[1-(3-bromoindazol-1-yl)cyclobutyl]acetonitrile (600 mg, 83% Yield) as a white solid. ¹H NMR (400 MHz, DMSO-d₆) δ 7.63 (d, J=9.1 Hz, 2H), 7.57-7.42 (m, 1H), 7.30 (dd, J=8.7, 7.0 Hz, 1H), 3.39 (s, 2H), 2.87 (dt, J=12.5, 9.7 Hz, 2H), 2.64-2.53 (m, 2H), 2.25-2.09 (m, 1H), 2.09-1.89 (m, 1H); m/z=290.0, 292.0 [M+H]+

### Step 2: Synthesis of 2-[1-[3-(4,4,5,5-tetramethyl-1,3,2-dioxaborolan-2-yl)indazol-1-yl]cyclobutyl]acetonitrile

A vial was charged with bis(pinacol)diborane (1.5 g, 6.00 mmol), potassium acetate (589 mg, 6.00 mmol), 2-[1-(3-bromoindazol-1-yl)cyclobutyl]acetonitrile (580 mg, 2.00 mmol), and bis(diphenylphosphino)ferrocene]dichloropalladium(II) (147 mg, 0.200 mmol) in anhydrous dioxane (20 mL). The vial was sealed, evacuated under vacuum and refilled with argon. The reaction mixture was stirred at 110° C. for 2 h then allowed to reach room temperature, filtered, washed with EtOAc, concentrated under vacuum to give crude material as a brown oil. The crude material was purified by flash chromatography on silica gel using a gradient of heptane/EtOAc. It was transferred via liquid injection in DCM on a 70 g column. Relevant fractions were collected and concentrated under vacuum to afford 2-[1-[3-(4,4,5,5-tetramethyl-1,3,2-dioxaborolan-2-yl)indazol-1-yl]cyclobutyl]acetonitrile as an orange solid.

1H NMR (400 MHz, DMSO-d6) δ 7.96 (d, J=8.1 Hz, 1H), 7.62 (d, J=8.5 Hz, 1H), 7.41-7.36 (m, 1H), 7.26-7.21 (m, 1H). 3.42 (s, 2H), 2.90 (dt, J=12.4, 9.8 Hz, 2H), 2.66-2.56 (m, 2H), 2.28-2.11 (m, 1H), 2.01-1.88 (m, 1H), 1.36 (s, 12H); m/z=256.3 [M+H]+

### Scaffold Coupling—General Procedure (Pyrazole)

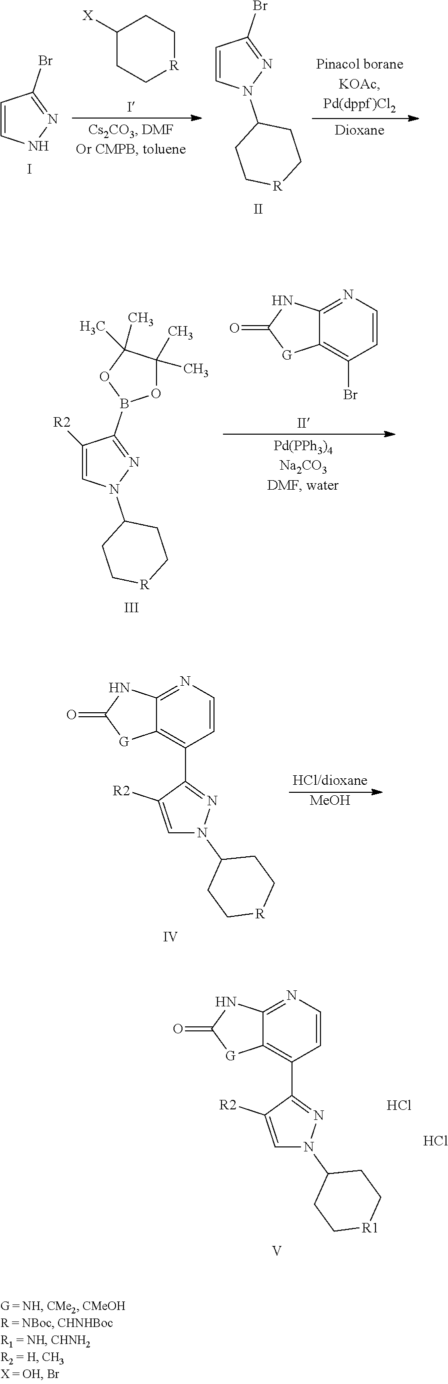

### First Step: Alkylation or Mitsunobu

1a. Alkylation (X═Br)

In a reactivial, cesium carbonate (532 mg, 1.63 mmol, 1.2 eq.) was added to a solution of 3-bromo-1H-pyrazole I (200 mg, 1.36 mmol) and tert-butyl 4-bromopiperidine-1-carboxylate (431 mg, 1.63 mmol, 1.2 eq.) in anhydrous DMF (14 mL, 0.1N). The mixture was heated to 70° C. overnight. tert-butyl 4-bromopiperidine-1-carboxylate (431 mg, 1.63 mmol, 2 eq.) and cesium carbonate (532 mg, 1.63 mmol, 2 eq.) were added and the mixture heated to 80° C. overnight. Tert-butyl 4-bromopiperidine-1-carboxylate (2 eq.) and cesium carbonate (2 eq.) were added again and the mixture heated to 80° C. for 4 hours more. Water was added and the mixture extracted with ethyl acetate. The organic phase was dried and concentrated under vacuum to afford crude material. The crude material was purified by flash chromatography on silica gel using a gradient of DCM/EtOAc. It was transferred via liquid injection in DCM. Relevant fractions were collected (not visible in UV) and concentrated under vacuum to afford tert-butyl 4-(3-bromopyrazol-1-yl)piperidine-1-carboxylate II (266 mg, 54% yield) as a pale oil. ¹H NMR (DMSO-d₆, 400 MHz): δ (ppm) 7.83 (d, J=2.4 Hz, 1H), 6.38 (d, J=2.3 Hz, 1H), 4.35 (tt, J=11.5, 4.0 Hz, 1H). 4.03 (d, J=12.3 Hz, 2H), 2.88 (s, 2H), 2.04-1.92 (m, 2H), 1.73 (qd, J=12.4, 4.4 Hz, 2H), 1.42 (s, 9H); m/z=276.1 [M+H]+

1b. Mitsunobu Reaction (X═OH)

Cyanomethylenetributylphosphorane (1.7 mL, 6.12 mmol, 3 eq.) was added to a solution of 3-bromo-1H-pyrazole I (300 mg, 2.04 mmol) and tert-butyl cis-4-hydroxycyclohexylcarbamate (1.32 g, 6.12 mmol, 3 eq.) in anhydrous toluene (10 mL, 0.2 N). The reaction mixture was stirred at 90° C. overnight. The solution was concentrated under vacuum. The crude material was purified by flash chromatography on silica gel using a gradient of heptane/EtOAc. Relevant fractions were collected and concentrated under vacuum to afford tert-butyl N-[4-(3-bromopyrazol-1-yl)cyclohexyl]carbamate II (288 mg, 41% yield) as a yellow solid. ¹H NMR (DMSO-d₆, 400 MHz): δ (ppm) 7.79 (d, J=2.3 Hz, 1H), 6.80 (d, J=7.9 Hz, 1H), 6.34 (d, J=2.3 Hz, 1H), 4.09 (tt, J=11.9, 3.9 Hz, 1H), 3.22-3.30 (m, 1H), 2.04-1.65 (m, 6H), 1.51-1.20 (m, 11H), m/z=288.1-290.1 [M+H-tBu]+

Same synthesis for following steps.

### 2. Boronic Esters

In a reactivial were introduced substituted bromopyrazoles II (0.806 mmol), bis(pinacolato)diboron (1.21 mmol, 1.5 eq.) and potassium acetate (2.42 mmol, 3 eq.) in anhydrous dioxane (2.7 mL, 0.3 N). The mixture was degassed with N₂ and bis(diphenylphosphino)ferrocene] dichloropalladium(II) (0.081 mmol, 0.1 eq.) was added. The reaction mixture was stirred at 100° C. overnight. The mixture was filtered on dicalite and concentrated under vacuum to give crude material III. The crude material was used in next step without further purification.

### Example: Synthesis of tert-butyl 4-[3-(4,4,5,5-tetramethyl-1,3,2-dioxaborolan-2-yl)pyrazol-1-yl]piperidine-1-carboxylate (R═NHBoc, R₂═H)

Dark oil, m/z=240.3 [M+H-tBu]+ (acid form)

### 3. Suzuki Coupling

A reacti-vial was charged with boronic esters III (0.709 mmol, 2 eq.), bromine scaffold I′ (0.332 mmol), disodium carbonate (0.996 mmol, 3 eq.) and tetrakistriphenylphosphine palladium (0.0332 mmol, 0.1 eq.) in a mixture of DMF (3.2 mL) and water (0.6 mL). The vial was degassed with nitrogen and stirred at 100° C. during 4 h. Water was added and the precipitate was filtered and solubilized in DCM. The organic phase was dried on a phase separator and concentrated under vacuum. The crude material was purified by flash chromatography on silica gel using a gradient of cyclohexane/EtOAc. Relevant fractions were collected and concentrated under vacuum to afford Suzuki coupling products IV.

### Example: Synthesis of Tert-Butyl 4-[3-(3,3-dimethyl-2-oxo-1H-pyrrolo[2,3-b]pyridin-4-yl)pyrazol-1-yl]piperidine-1-carboxylate (R═NHBoc, R₂═H, G=CMe₂)

Yellow solid, 54% yield, ¹H NMR (DMSO-d₆, 400 MHz): δ (ppm) 11.01 (s, 1H), 8.07 (d, J=5.5 Hz, 1H), 7.95 (d, J=2.4 Hz, 1H), 7.24 (d, J=5.6 Hz, 1H), 6.83 (d, J=2.4 Hz, 1H), 4.49 (ddt, J=11.4, 7.9, 4.0 Hz, 1H), 4.13-3.99 (m, 2H), 2.91 (d, J=14.5 Hz, 2H), 2.06 (d, J=10.0 Hz, 2H), 1.86 (qd, J=12.4, 4.2 Hz, 2H), 1.45 (d, J=14.7 Hz, 15H); m/z=412.4 [M+H]+

### 4. Deprotection

4 M hydrogen chloride solution in dioxane (0.73 mmol, 4 eq.) was added to a solution of Suzuki coupling compounds IV (0.18 mmol) in anhydrous methanol (0.16 mL, 0.1 N). The reaction was stirred at room temperature overnight. The solution was concentrated under vacuum. The product was triturated in DCM and dried under vacuum at 40° C. overnight to give final compounds IV as hydrochloric salts.

### Example: Synthesis of 3,3-dimethyl-4-[1-(4-piperidyl)pyrazol-3-yl]-1H-pyrrolo[2,3-b]pyridin-2-one Dihydrochloride (R═NH, R₂═H, G=CMe₂)

White powder, 73% yield, 1H NMR (DMSO-d6, 500 MHz): δ (ppm) 11.06 (s, 1H), 8.62-9.21 (m, 2H), 8.08 (d, J=5.6 Hz, 1H), 7.93 (d, J=2.4 Hz, 1H), 7.25 (d, J=5.6 Hz, 1H), 6.86 (d, J=2.4 Hz, 1H), 4.59 (tt, J=10.3, 5.0 Hz, 1H), 4.25 (br s, 1H), 3.43 (br d, J=13.0 Hz, 2H), 3.02-3.14 (m, 2H), 2.13-2.29 (m, 4H), 1.48 (s, 6H); m/z=312.1 [M+H]+

### Scaffold Coupling—General Procedure (Pyrrole)

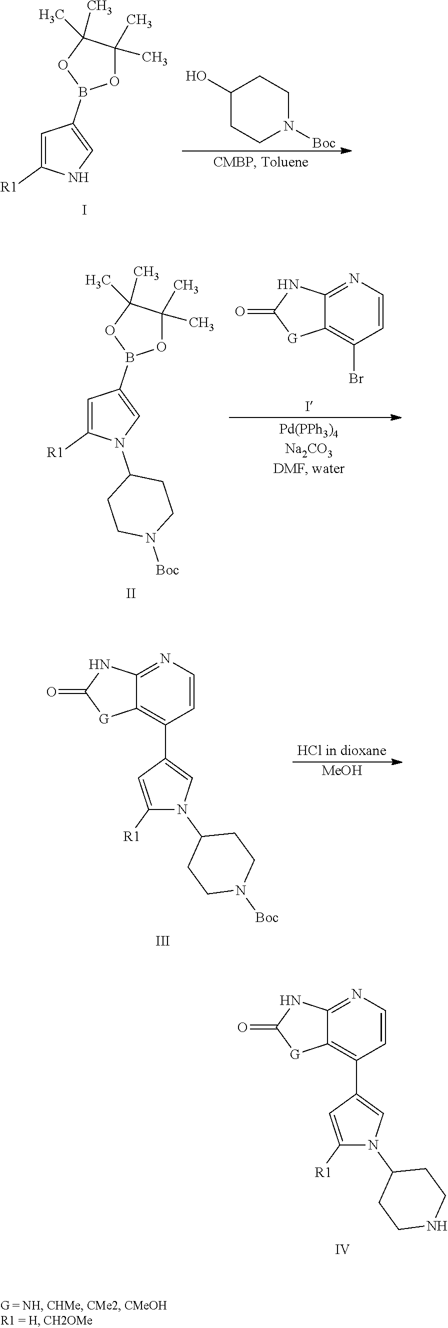

### 1. Mitsunobu Reaction

A 10 mL reacti-vial was charged with 3-(4,4,5,5-tetramethyl-1,3,2-dioxaborolan-2-yl)-1H-pyrrole (200 mg, 1.02 mmol) I, tert-butyl 4-hydroxypiperidine-1-carboxylate (409 mg, 2.03 mmol, 2 eq.), and anhydrous toluene (5 mL, 0.2 N). The vial was sealed and cyanomethylenetributylphosphorane (0.55 mL, 2.03 mmol, 2 eq.) was added. The reaction mixture was stirred at 110° C. during 3 h. tert-butyl 4-hydroxypiperidine-1-carboxylate (409 mg, 2.03 mmol, 2 eq.) and cyanomethylenetributylphosphorane (0.55 mL, 2.03 mmol, 2 eq.) were added and the reaction mixture was stirred at 110° C. for 4 h. Tert-butyl-4-hydroxypiperidine-1-carboxylate (2 eq) and cyanomethylenetributylphosphorane (2 eq) were added again and the reaction mixture was stirred at 110° C. overnight. The reaction was stopped and the solvent was removed under vacuum. The crude material was purified by reverse chromatography in neutral conditions (MeCN/water). Relevant fractions were collected and concentrated under vacuum to afford tert-butyl 4-[3-(4,4,5,5-tetramethyl-1,3,2-dioxaborolan-2-yl)pyrrol-1-yl]piperidine-1-carboxylate (106 mg, 28%) as a white solid. ¹H NMR (DMSO-d₆, 400 MHz): δ (ppm) 7.14 (t, J=1.7 Hz, 1H), 6.88 (t, J=2.3 Hz, 1H), 6.21-6.17 (m, 1H), 4.16-3.95 (m, 3H), 2.82 (s, 2H), 1.91 (d, J=12.5 Hz, 2H), 1.76-1.59 (m, 2H), 1.42 (s, 9H), 1.23 (s, 12H); m/z=377.3 [M+H]+

### 2. Suzuki Coupling

A reacti-vial was charged with tert-butyl 4-[3-(4,4,5,5-tetramethyl-1,3,2-dioxaborolan-2-yl)pyrrol-1-yl]piperidine-1-carboxylate II (0.282 mmol, 1.1 eq.), bromine scaffold I′ (0.256 mmol), disodium carbonate (81 mg, 0.768 mmol) and tetrakistriphenylphosphine palladium (30 mg, 0.0256 mmol, 0.1 eq.) in a mixture of DMF (2.5 mL) and water (0.5 mL). The vial was sealed, degassed with nitrogen and stirred at 100° C. overnight. The reaction was stopped and the reaction mixture was filtered through a dicalite pad, then washed with DCM. The solvent was removed under vacuum to give crude material. The crude material was purified by flash chromatography on silica gel using a gradient of cyclohexane/EtOAc followed by a gradient of toluene/acetone. Relevant fractions were collected and concentrated under vacuum to afford expected products III.

### Example: Synthesis of Tert-Butyl 4-[3-(2-oxo-1,3-dihydroimidazo[4,5-b]pyridin-7-yl)pyrrol-1-yl]piperidine-1-carboxylate (G=NH)

White solid; Yield 13%; ¹H NMR (DMSO-d₆, 400 MHz): δ (ppm) 11.22 (s, 1H), 10.63 (s, 1H), 7.86 (dd, J=5.2, 1.4 Hz, 1H), 7.79 (d, J=5.6 Hz, 1H), 7.66 (s, 1H), 7.12 (d, J=5.6 Hz, 1H), 6.59 (s, 1H), 4.12 (s, 3H), 2.86 (s, 2H), 2.05-1.69 (m, 4H), 1.44 (s, 9H); m/z=384.5 [M+H]+

### 3. Deprotection

4 M hydrogen chloride solution in dioxane (0.36 mmol, 10 eq.) was added to a solution of Suzuki coupling compounds III (0.032 mmol) in anhydrous methanol (0.16 mL, 0.2 N). The reaction was stirred at room temperature overnight. The reaction mixture was filtered and the product washed with pentane and dried under vacuum at 40° C. to give final compounds as hydrochloric salts.

### Example 6: Synthesis of 7-[1-(4-piperidyl)pyrrol-3-yl]-1,3-dihydroimidazo[4,5-b]pyridin-2-one; Dihydrochloride (G=NH)

White powder; yield 50%, 1H NMR (DMSO-d₆, 500 MHz): δ (ppm) 11.77 (br s, 1H), 10.97 (br s, 1H), 9.08 (br s, 1H), 8.89 (br s, 1H), 7.82 (d, J=5.9 Hz, 1H), 7.64 (s, 1H), 7.22 (d, J=5.9 Hz, 1H), 7.00 (t, J=2.2 Hz, 1H), 6.70 (br s, 1H), 4.28 (tt, J=11.4, 3.6 Hz, 1H), 3.53 (br s, 1H), 3.36-3.46 (m, 2H), 3.05 (q, J=12.1 Hz, 2H), 2.23 (br d, J=12.5 Hz, 2H), 2.06-2.18 (m, 2H); m/z=284.2 [M+H]+

### Example 2—Biological Assays

### PKC-Theta and PKC-Delta Inhibition Assay

PKC-theta and PKC-delta biochemical activities were measured using the PKC-theta HTRF KinEASEkit kit, according to manufacturer's instructions (Cisbio, catalogue number 61ST1PEJ). Briefly, the kinase buffer component of the kit was supplemented with 10 mM MgCl2, 1 mM DTT and 0.1% Tween 20. For the PKC-theta assay, STK substrate and ATP were added to provide a final assay concentration of 525 nM and 6.5 μM, respectively. For the PKC-delta assay, STK substrate and ATP were added to provide a final assay concentration of 243 nM and 5.7 μM, respectively. The streptavidin_XL665 and STK antibody-cryptate detection reagents were mixed according to the manufacturer's instructions. Test compounds were diluted in DMSO in a series of 10 semi-log step doses; 10 nL of each compound dose were dispensed in 384 well plates. Recombinant human PKC-theta (His-tagged 362-706) or PKC-delta (His-tagged 345-676) was diluted into kinase buffer to provide a final assay concentration of 10 ng/ml and added to the test compound for 30 minutes on ice. The reaction was started by addition of the substrate and ATP and incubated at 25° C. for 30 minutes or 20 minutes for the PKC-theta and PKC-delta assays, respectively. The detection reagents were added, and the plate was incubated in the dark for 2 hours. Fluorescence was measured on an Envision 2103 plate reader with optical setup for excitation at 665 nM and emission at 620 nM in the HTRF mode. The ratio of acceptor and donor emission signals was calculated for each well. Percent inhibition values were calculated from the HTRF ratios at different doses and fitted to a 4-parameter logistic curve to determine IC₅₀ values (see Table 1).

### Effector Memory T Cells IL-2 Release Assay

Test compound-mediated inhibition of NFκB signalling in T cells was assessed by quantification of the IL-2 secretion by human effector memory T cells (TEM) upon treatment and stimulation. Human TEM cells were isolated from buffy coats of healthy donors obtained from the French blood bank. First, peripheral blood mononuclear cells (PBMC) were purified from buffy coats diluted 1:1 with DPBS (Gibco, cat #14190-094) by Pancoll (PAN BIOTECH, cat #P04-60500) density gradient centrifugation at 400×g for 20 minutes. TEM cells were further enriched by negative immuno-magnetic cell sorting using a human CD4+ Effector Memory T Cell Isolation Kit (Miltenyi, cat #130-094-125) according to the manufacturer's instructions. Aliquots of 3×10E6 purified TEM cells were kept frozen in Cryo-SFM medium (PromoCell, cat #C-29912) in gas phase nitrogen until used. Cell purity was verified by flow cytometry analysis of 200 000 PFA-fixed cells previously labelled with monoclonal antibodies anti-CD4-PerCP-Cy5.5 (BD Pharmigen, cat #332772), anti-CD8-V500 (BD Biosciences, cat #561617), anti-CD14-Pacific Blue (Biolegend, cat #325616), anti-CD45 RA-FITC (Biolegend, cat #304106) and anti-CCR7-APC (in CD4+ Effector Memory T Cell Isolation Kit, Miltenyi, cat #130-094-125).

TEM cells were resuspended in complete RPMI medium composed of: RPMI 1640 (Gibco, cat #31870-025), 10% heat inactivated fetal bovine serum (Sigma, cat #F7524), 2 mM GlutaMAX (Gibco, cat #35050-038), 1 mM sodium pyruvate 100× (Gibco, cat #11360-039), 1% MEM non-essential amino acids solution (Gibco, cat #11140-035) and 100 U/mL penicillin, 100 g/mL streptomycin (Sigma-Aldrich, cat #11074440001). 5,000 cells per well were plated onto flat clear bottom 384 well plates (Corning, cat #3770). 5,000 Dynabeads Human T-Activator CD3/CD28 (Gibco, cat #11132D) were added to each well for cell stimulation. Finally, 10 doses of test compound, originally prepared in DMSO by serial semi-log step dilution, were also added to cells in triplicate wells. Final DMSO concentration in wells was 0.1% in a total volume of 100 UL complete medium. Plates were incubated for 24 h at 37° C. in 5% CO₂ atmosphere. After incubation, cell suspensions were centrifuged at 400×g and culture supernatants were recovered and stored at −80° C. Cell viability was assessed by flow cytometry after staining the cells with Fixable Viability Dye eFluor 780 (Invitrogen, cat #65-0865-14). IL-2 levels were determined in cell supernatants using an HTRF human IL-2 detection kit (Cisbio, cat #62HIL02PEH). IL-2 data at the different compound doses were fitted to a 4-parameter logistic curve to determine IC₅₀ values, corresponding to the compound concentration leading to 50% reduction of the maximal IL-2 levels observed in each experiment. Viability data were analysed similarly to exclude cytotoxicity as a cause of IL-2 decrease (see Table 1).

TABLE 2

| | | | | | | PKC- |
|---|---|---|---|---|---|---|
| | | PKC- | | | PKC- | theta/ |
| | PKC- | theta | | | theta/ | PKC- |
| | theta | HTRF | | PKC-theta | PKC- | delta |
| Example | HTRF | pIC50 | PKC-theta | IL2 pIC50 | delta | selectivity |
| no. | pIC50 | (binned) | IL2 pIC50 | (binned) | selectivity | (binned) |
| 1 | 6.1 | G | 5.5 | G | 1 | G |
| 2 | 5.1 | H | 5.0 | G | 0.2 | G |
| 3 | 6.7 | F | 5.2 | G | 1 | F |
| 4 | 6.0 | H | 5.4 | G | 2 | F |
| 5 | 7.1 | E | 6.9 | E | 1 | F |
| 6 | 7.4 | E | 6.2 | F | 3 | F |
| 7 | 7.4 | E | 6.3 | F | 6 | E |
| 8 | 8.1 | C | 6.9 | E | 5 | E |
| 9 | 8.7 | B | 7.4 | D | 15 | D |
| 10 | 7.4 | E | 6.2 | F | 12 | D |
| 11 | 7.2 | E | 5.8 | G | 4 | F |
| 12 | 8.1 | C | 6.7 | E | 11 | D |
| 13 | 7.4 | E | 6.2 | F | 4 | F |
| 14 | 8.5 | C | 7.3 | D | 3 | F |
| 15 | 8.6 | B | 7.4 | D | 14 | D |
| 16 | 8.3 | C | 7.2 | D | 18 | D |
| 17 | 8.0 | D | 6.4 | F | 7 | E |
| 18 | 7.9 | D | 6.5 | F | 11 | D |
| 19 | 8.4 | C | 7.3 | D | 9 | E |
| 20 | 7.8 | D | 6.7 | E | 13 | D |
| 21 | 7.3 | E | 6.0 | G | 9 | E |
| 22 | 8.0 | C | 6.5 | F | 15 | D |
| 23 | 9.3 | A | 7.5 | C | 11 | D |
| 24 | 8.4 | C | 7.2 | D | 9 | E |
| 25 | 9.3 | A | 7.7 | C | 6 | E |
| 26 | 8.5 | C | 7.1 | D | 11 | D |
| 27 | 7.3 | E | 5.8 | G | 5 | E |
| 28 | 8.7 | B | 7.5 | C | 15 | D |
| 29 | 9.4 | A | 7.4 | D | 24 | C |
| 30 | 8.9 | B | 7.3 | D | 14 | D |
| 31 | 8.9 | B | 7.4 | D | 22 | C |
| 32 | 8.1 | C | 7.0 | E | 13 | D |
| 33 | 8.5 | B | 7.2 | D | 22 | C |
| 34 | 8.8 | B | 7.4 | D | 12 | D |
| 35 | 8.5 | C | 7.0 | D | 11 | D |
| 36 | 8.3 | C | 6.9 | E | 14 | D |
| 37 | 8.6 | B | 7.6 | C | 19 | D |
| 38 | 8.9 | B | 7.7 | C | 16 | D |
| 39 | 9.7 | A | 8.0 | B | 10 | E |
| 40 | 9.7 | A | 7.8 | C | 17 | D |
| 41 | 9.4 | A | 7.8 | C | 14 | D |
| 42 | 7.6 | D | 6.4 | F | N/D | N/D |
| 43 | 8.1 | C | 6.5 | F | 11 | D |
| 44 | 7.5 | E | 6.6 | E | 14 | D |
| 45 | 7.4 | E | 6.0 | G | 15 | D |
| 46 | 8.2 | C | 6.8 | E | 3 | F |
| 47 | 9.2 | A | 7.6 | C | 12 | D |
| 48 | 6.5 | F | 6.1 | F | 33 | B |
| 49 | 8.2 | C | 6.5 | E | 3 | F |
| 50 | 9.1 | A | 7.7 | C | 11 | D |
| 51 | 9.3 | A | 7.8 | C | 11 | D |
| 52 | 8.4 | C | 7.4 | D | 8 | E |
| 53 | 8.3 | C | 7.0 | D | 13 | D |
| 54 | 8.6 | B | 7.1 | D | 22 | C |
| 55 | 8.2 | C | 6.8 | E | 4 | F |
| 56 | 8.7 | B | 7.3 | D | 15 | D |
| 57 | 8.9 | B | 7.3 | D | 14 | D |
| 58 | 9.3 | A | 8.0 | C | 18 | D |
| 59 | 9.2 | A | 7.7 | C | 11 | D |
| 60 | 8.4 | C | 7.0 | E | 8 | E |
| 61 | 8.7 | B | 7.3 | D | 16 | D |
| 62 | 9.5 | A | 8.8 | A | 3 | F |
| 63 | 7.5 | D | 5.9 | G | 17 | D |
| 64 | 8.6 | B | 7.1 | D | 28 | C |
| 65 | 7.3 | E | 6.4 | F | 28 | C |
| 66 | 8.4 | C | 7.0 | D | 21 | C |
| 67 | 7.5 | E | 6.4 | F | 9 | E |
| 68 | 9.6 | A | 8.0 | C | 12 | D |
| 69 | 7.0 | E | 5.9 | G | 9 | E |
| 70 | 8.1 | C | 6.9 | E | 20 | C |
| 71 | 8.1 | C | 6.9 | E | 22 | C |
| 72 | 7.9 | D | 6.7 | E | 20 | D |
| 73 | 7.5 | D | 6.2 | F | 10 | D |
| 74 | 6.5 | F | 6.2 | F | 33 | B |
| 75 | 8.7 | B | 7.3 | D | 19 | D |
| 76 | 8.5 | B | 7.0 | D | 22 | C |
| 77 | 9.0 | A | 7.9 | C | 11 | D |
| 78 | 9.0 | A | 7.3 | D | 10 | D |
| 79 | 8.9 | B | 7.3 | D | 15 | D |
| 80 | 9.1 | A | 7.7 | C | 9 | E |
| 81 | 7.5 | E | 6.1 | F | 20 | C |
| 82 | 9.1 | A | 7.3 | D | 12 | D |
| 83 | 8.8 | B | 7.2 | D | 13 | D |
| 84 | 7.5 | E | 5.9 | G | 14 | D |
| 85 | 7.3 | E | 5.9 | G | 10 | E |
| 86 | 7.5 | D | 5.9 | G | 26 | C |
| 87 | 9.0 | A | 7.3 | D | 38 | B |
| 88 | 8.4 | C | 6.7 | E | 14 | D |
| 89 | 8.6 | B | 7.1 | D | 32 | B |
| 90 | 9.0 | A | 7.8 | C | 11 | D |
| 91 | 8.1 | C | 6.9 | E | 19 | D |
| 92 | 7.9 | D | 6.8 | E | 15 | D |
| 93 | 8.4 | C | 7.3 | D | 10 | E |
| 94 | 8.4 | C | 6.9 | E | 23 | C |
| 95 | 9.9 | A | 8.8 | A | 25 | C |
| 96 | 8.7 | B | 7.6 | C | 17 | D |
| 97 | 8.6 | B | 7.4 | D | 23 | C |
| 98 | 7.5 | D | 6.0 | G | 57 | A |
| 99 | 7.0 | E | 6.4 | F | 105 | A |
| 100 | 8.1 | C | 7.0 | D | 29 | C |
| 101 | 7.6 | D | 6.2 | F | 42 | B |
| 102 | 8.2 | C | N/D | N/D | 46 | B |
| 103 | 8.6 | B | 7.2 | D | 22 | C |
| 104 | 9.1 | A | 7.9 | C | 17 | D |
| 105 | 7.9 | D | 6.4 | F | 33 | B |
| 106 | 9.1 | A | 7.9 | C | 15 | D |
| 107 | 7.7 | D | 6.2 | F | 23 | C |
| 108 | 8.5 | B | 7.3 | D | 17 | D |
| 109 | 7.7 | D | 6.3 | F | 11 | D |
| 110 | 7.6 | D | 6.0 | G | 29 | C |
| 111 | 8.0 | D | 6.4 | F | 7 | E |
| 112 | 8.5 | B | 7.2 | D | 22 | C |

Biochemical data for representative compounds of the disclosure. In the columns indicated, the data has been binned in a category of A to H as indicated below dependent on the measured value.

For PKC-theta HTRF:
A means a measured pIC50 of 9.0 and above;
B means a measured pIC50 of between 8.5 and 9.0;
C means a measured pIC50 of between 8.0 and 8.5;
D means a measured pIC50 of between 7.5 and 8.0;
E means a measured pIC50 of between 7.0 and 7.5;
F means a measured pIC50 of between 6.5 and 7.0;
G means a measured pIC50 of between 6.0 and 6.5;
H means a measured pIC50 of <6.0.

For PKC-theta CD4Tc IL-2:
A means a measured pIC50 of between 8.5 and 9.0;
B means a measured pIC50 of between 8.0 and 8.5;
C means a measured pIC50 of between 7.5 and 8.0;
D means a measured pIC50 of between 7.0 and 7.5;
E means a measured pIC50 of between 6.5 and 7.0;
F means a measured pIC50 of between 6.0 and 6.5;
G means a measured pIC50 of <6.0.

For PKC-theta/PKC-delta selectivity:
A means a ratio of between 50 and 120;
B means a ratio of between 30 and 50;
C means a ratio of between 20 and 30;
D means a ratio of between 10 and 20;
E means a ratio of between 5 and 10;
F means a ratio of between 1 and 5;
G means a ratio of between 0 and 1.

Modifications may be made to the above examples without departing from the scope of the present invention as defined in the accompanying claims.

1. A compound having the structural Formula I: 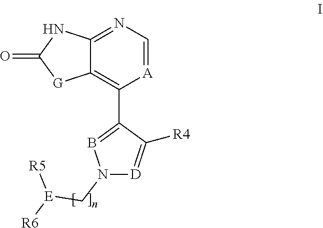
or a pharmaceutically acceptable salt, solvate, stereoisomer or mixture of stereoisomers, tautomer, or isotopic form, or pharmaceutically active metabolite thereof, or combinations thereof, wherein:
A is selected from the group consisting of: N, C—R^{a}, where R^{a} is selected from hydrogen, halogen, C1-3 alkyl and CN;
G is selected from the group consisting of: CR1R2; O and NR1;
R1 and R2 are independently selected from the group consisting of: hydrogen, halogen, C1-3 alkyl; C3-7 cycloalkyl; C1-3 alkoxyl; C2-6 cycloalkoxyl; C2-6 alkyl alkoxy; hydroxyl, C1-3 alkyl hydroxyl; amino, C1-3 alkyl amino; C1-4 amino alkyl; C2-7 alkyl amino alkyl; and C1-3 haloalkyl; or
R1 and R2 together form a 3-5 membered optionally substituted spiro carbocyclic or heterocyclic ring;
B is selected from the group consisting of: N; C—H and C-halogen;
D is selected from the group consisting of: N; and C—R3;
R3 is selected from the group consisting of: hydrogen; C1-3 alkyl; C1-3 halo alkyl; C1-3 alkoxy; C2-5 alkyl alkoxy; and halogen;
R4 is selected from the group consisting of: hydrogen; C1-3 alkyl; C1-3 halo alkyl; OMe; and halogen; or
wherein when D is C—R3, R3 and R4 together are joined to form an optionally substituted aryl or heteroaryl ring having the structure selected from the group consisting of: 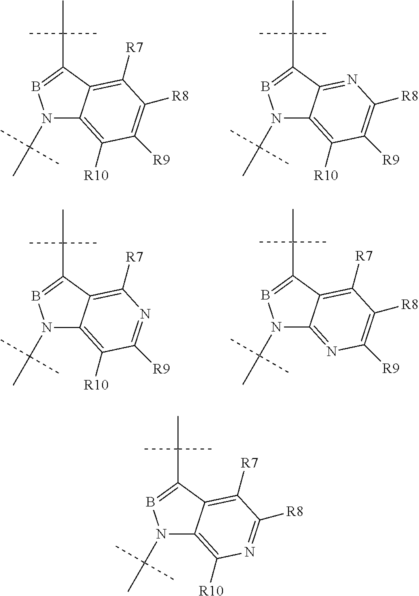
wherein;
R7 is selected from the group consisting of: hydrogen; and halogen;
R8 is selected from the group consisting of: hydrogen; and halogen;
R9 is selected from the group consisting of: hydrogen; C1-3 halo alkyl; and halogen;
R10 is selected from the group consisting of: hydrogen; halogen; C1-3 halo alkyl; C1-3 haloalkoxy; and wherein:
n is selected from the group consisting of: 0; and 1;
E is selected from the group consisting of: C—H; and C—R^{a}, where R^{a} is selected from halogen; C1-3 alkyl; C1-3 alkyl hydroxy; C1-3 haloalkyl; C2-6 alkyl alkoxy and C1-3 alkyl nitrile;
R5 and R6 are Joined together to form an optionally substituted, optionally bridged, 4-8-membered saturated carbocyclic or heterocyclic ring.

2. The compound of claim 1, having the structural Formula II:

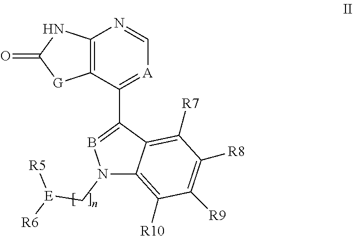

3. The compound of claim 1 or claim 2, having the structural Formula IIa: 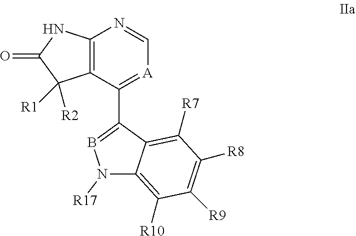
wherein, R17 is selected from the group consisting of: 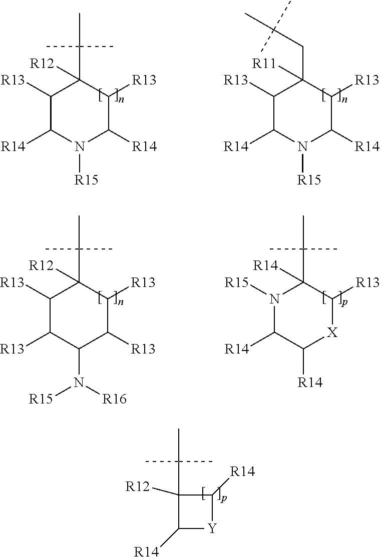
wherein
R11 is selected from the group consisting of: hydrogen; halogen and C1-2 alkyl;
R12 is selected from the group consisting of: hydrogen; C1-3 alkyl; C1-3 haloalkyl; C1-3 alkyl hydroxyl; and C1-2 alkyl nitrile;
R13 is selected from the group consisting of: hydrogen; halogen and C1-2 alkyl;
R14 is selected from the group consisting of: hydrogen and C1-2 alkyl;
R15 is selected from the group consisting of: hydrogen and C1-2 alkyl;
R16 is selected from the group consisting of: hydrogen; C1-3 alkyl; C1-3 haloalkyl; C1-3 alkyl hydroxy; and C1-3 alkyl alkoxy;
n is selected from the group consisting of: 0 and 1;
p is selected from the group consisting of: 1 and 2;
X is selected from the group consisting of: CH₂ and O;
Y is selected from the group consisting of: CH₂; O; NH and NMe.

4. The compound of claim 3, wherein:
R1 is selected from the group consisting of: hydrogen, Me, Et, OMe, OEt, OH, NH₂, NHMe and NHEt;
R2 is selected from the group consisting of: hydrogen, Me and Et; or
R1 and R2 together form a 3-5 membered optionally substituted spiro carbocyclic or heterocyclic ring; particularly a 4-5 membered optionally substituted carbocyclic or heterocyclic spiro ring; wherein in embodiments the carbocyclic or heterocyclic spiro ring is unsubstituted; wherein in alternative embodiments the carbocyclic or heterocyclic spiro ring is substituted with one or more substituents selected from the group consisting of: C1-2 alkyl, halogen; C1-2 haloalkyl; hydroxyl; and C1-2 alkoxyl;
A is selected from the group consisting of: C—H; C—F; C—Cl and C—Br;
B is selected from the group consisting of: N; C—H, C—F; C—Cl and C—Br;
R17 is selected from the group consisting of: 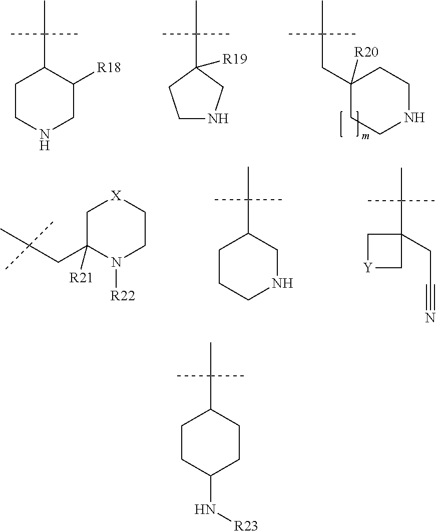
wherein:
R18 is selected from the group consisting of: hydrogen; and halogen;
R19 is selected from the group consisting of: hydrogen; C1-3 alkyl; C1-3 haloalkyl; C1-3 alkyl hydroxy;
m is selected from the group consisting of: 0 and 1;
R20 is selected from the group consisting of: hydrogen; halogen;
X is selected from the group consisting of: CH₂ and O;
R21 and R22 are each independently selected from the group consisting of: hydrogen and C1-3 alkyl;
Y is selected from the group consisting of: CH₂; O and NH;
R23 is selected from the group consisting of: hydrogen; C1-3 alkyl; C1-3 haloalkyl.

5. The compound of claim 1, having the structural Formula III: 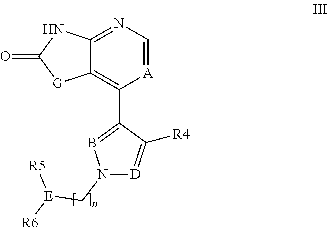
wherein;
D is selected from the group consisting of: N; C—H and C—R3;
R3 is selected from the group consisting of: C1-3 alkyl; C2-5 alkyl alkoxy; C1-3 haloalkyl and halogen;
R4 is selected from the group consisting of: hydrogen; C1-3 alkyl; C2-5 alkyl alkoxyl; C1-3 haloalkyl and halogen.

6. The compound of claim 5, having the structural Formula IIIa, IIIb or IIIc: 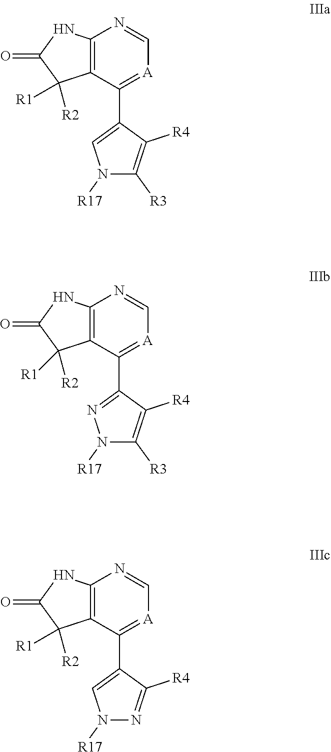
wherein,
R17 is selected from the group consisting of: 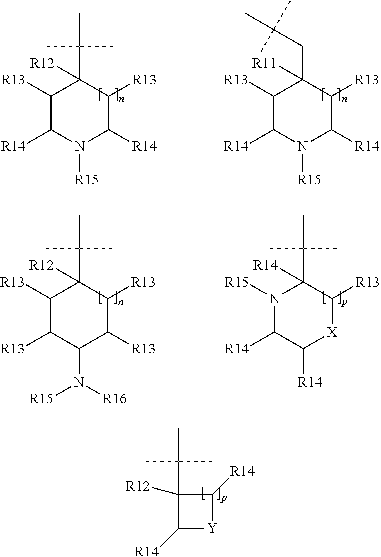
wherein
R11 is selected from the group consisting of: hydrogen; halogen and C1-2 alkyl;
R12 is selected from the group consisting of: hydrogen; C1-3 alkyl; C1-3 haloalkyl; C1-3 alkyl hydroxyl; and C1-2 alkyl nitrile;
R13 is selected from the group consisting of: hydrogen; halogen and C1-2 alkyl;
R14 is selected from the group consisting of: hydrogen and C1-2 alkyl;
R15 is selected from the group consisting of: hydrogen and C1-2 alkyl;
R16 is selected from the group consisting of: hydrogen; C1-3 alkyl; C1-3 haloalkyl; C1-3 alkyl hydroxyl; and C1-3 alkyl alkoxyl;
n is selected from the group consisting of: 0 and 1;
p is selected from the group consisting of: 1 and 2;
X is selected from the group consisting of: CH₂ and O;
Y is selected from the group consisting of: CH₂, O, NH and NMe.

7. The compound of claim 6, wherein:
R1 is selected from the group consisting of: hydrogen, Me, Et, OMe, OH, NH₂, and NHMe;
R2 is selected from the group consisting of: hydrogen; Me; and Et; or
R1 and R2 together form a 3-5 membered optionally substituted spiro carbocyclic or heterocyclic ring;
A is selected from the group consisting of: C—H, C—F, C—Cl and C—Br;
R17 is selected from the group consisting of: 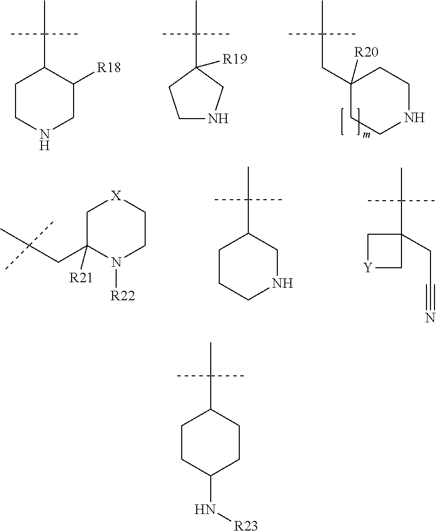
wherein:
R18 is selected from the group consisting of: hydrogen and halogen;
R19 is selected from the group consisting of: hydrogen; C1-3 alkyl; C1-3 haloalkyl; C1-3 alkyl and hydroxyl;
m is selected from the group consisting of: 0 and 1;
R20 is selected from the group consisting of: hydrogen and halogen;
X is selected from the group consisting of: CH₂; and O;
R21 and R22 are each independently selected from the group consisting of: hydrogen and C1-3 alkyl;
Y is selected from the group consisting of: CH₂; O and NH;
R23 is selected from the group consisting of: hydrogen; C1-3 alkyl; and C1-3 haloalkyl.

8. A compound according to Table 1, or a pharmaceutically acceptable salt, solvate, stereoisomer or mixture of stereoisomers, tautomer, isotopic form, or pharmaceutically active metabolite thereof, or combinations thereof.

9. A pharmaceutical composition comprising a compound of any of claims 1 to 8 or a pharmaceutically acceptable salt, solvate, stereoisomer or mixture of stereoisomers, tautomer, or isotopic form, or pharmaceutically active metabolite thereof, or combinations thereof, and one or more pharmaceutically acceptable carrier.

10. The compound of any of claims 1 to 8 or the pharmaceutical composition of claim 9 for use in the treatment of a disorder or disease selected from autoimmune disorders and/or inflammatory diseases and/or oncologic disease and/or cancers and/or HIV infection and replication.

11. The compound or pharmaceutical composition for use according to claim 10, wherein the disorder or disease is selected from the group consisting of: rheumatoid arthritis, multiple sclerosis, psoriasis, atopic dermatitis.

12. The compound or pharmaceutical composition for use according to claim 10 or claim 11, wherein the compound is an inhibitor of PKC-theta.

13. The compound or pharmaceutical composition for use according to any of claims 10 to 12, wherein the use is in a method comprising administering the compound orally, topically, by inhalation, by intranasal administration, or systemically by intravenous, intraperitoneal, subcutaneous, or intramuscular injection.

14. The compound or pharmaceutical composition for use according to any of claims 10 to 13, wherein the use is in a method comprising administering the compound according to any one of claims 1 to 8 in combination with one or more additional therapeutic agent.

15. The compound or pharmaceutical composition for use according to claim 14, wherein the administering comprises administering the compound according to any one of claims 1 to 8 simultaneously, sequentially or separately from the one or more additional therapeutic agents.

16. The compound or pharmaceutical composition for use according to any of claims 10 to 15, which comprises administering to a subject an effective amount of the compound according to any one of claims 1 to 8, wherein the effective amount is between about 5 nM and about 10 μM in the blood of the subject.
